(19) United States
(12) Patent Application Publication
(10) Pub. No.: US 2024/0426732 A1
(43) Pub. Date: Dec. 26, 2024

(54) METHOD FOR DETERMINING STATIC AND DYNAMIC ELASTIC AND POROELASTIC PARAMETERS OF ROCKS UNDER HYDROSTATIC CONFINING PRESSURE CONDITIONS, SYSTEM FOR CARRYING OUT SAID METHOD AND COMPUTER-READABLE STORAGE MEDIUM

(71) Applicant: PETRÓLEO BRASILEIRO S.A. - PETROBRAS, Rio de Janeiro (BR)

(72) Inventors: Marcos Pozzato Figueiredo, Rio de Janeiro (BR); Guilherme Fernandes Vasquez, Rio de Janeiro (BR); Julio Cesar Ramos Justen, Rio de Janeiro (BR)

(21) Appl. No.: 18/639,039

(22) Filed: Apr. 18, 2024

(30) Foreign Application Priority Data
Apr. 18, 2023 (BR) 10 2023 0072682

(51) Int. Cl. G01N 15/08; G01N 3/12; G01N 29/04; G01N 29/07; G01N 33/24

(52) U.S. Cl. CPC G01N 15/0806; G01N 3/12; G01N 15/082; G01N 29/043; G01N 29/07; G01N 33/24; G01N 2291/02827

(57) ABSTRACT

The present invention pertains to the field of modeling, simulation and evaluation of reservoirs and discloses preferred embodiments of a method for determining static and dynamic elastic and poroelastic parameters of rocks under hydrostatic confining pressure conditions, a system for carrying out said method and a computer-readable storage medium.

### RELATED APPLICATIONS

This application claims the benefit of Brazilian Patent Application No. BR 1020230072682, filed Apr. 18, 2023, the entire contents of which are explicitly incorporated by reference herein

### FIELD

The present invention pertains to the field of modeling, simulation and evaluation of reservoirs. More specifically, the present invention relates to the determination of static and dynamic elastic and poroelastic parameters of rocks under hydrostatic confining pressure conditions.

### BACKGROUND

Large volumes of oil and gas produced in the world by industry are stored in rocks (with adequate porosity and permeability) and such rocks are called reservoirs. Knowing more precisely the mechanical behavior of the rocks that make up the reservoir (and adjacent rocks) is becoming increasingly important to increase the economy and safety of the oil production process.

To characterize this behavior, the elastic and poroelastic properties of the rocks are necessary parameters to accurately model the mechanical response due to pressure changes during production, and these parameters are used in coupled and uncoupled poromechanical analyses.

During oil production, the pressure of the fluids within the reservoir changes throughout this process and can lead to a crucial impact on the economic results of oil production, and the possible impacts should be the compaction of the reservoirs and subsidence, as illustrated in FIG. 1.1 [1].

To evaluate these effects, one of the most relevant poroelastic properties of the reservoir rocks that must be known in advance is the value of pore compressibility, as it represents changes in the volumetric compression modulus associated with variations in pore pressure, and is used as an input into numerical simulations in reservoir flow models, for example.

To understand pore compressibility, the theory of linear poroelasticity was developed and has been equally important in groundwater problems, civil engineering and geotechnical engineering. Geophysics also benefits from poroelastic data from rock physics investigations [2].

Therefore, knowledge of the compressibility of pores, in a more refined way, implies better predictability of the behavior of oil reservoir rocks, as well as their overlayer and insufficient load behavior. Such parameters can also be used to evaluate the results of 4D seismic, a geophysical technique used to monitor the behavior of reservoirs over the production time.

Elastic and poroelastic parameters can be determined in several ways, depending on the level of information required and the availability of suitable samples for laboratory testing. Some methods involve testing analogous materials, based on test samples from different lithologies, but not necessarily in the field of interest. These methods are useful for obtaining the parameter as a first approach, but should be avoided when developing reservoir production prediction studies, as they generally do not have sufficient precision.

Therefore, it is necessary to obtain values from laboratory tests with samples from the region of interest to obtain different compressibility of rocks, exposing them to confining pressure and pore pressure in a controlled manner, then calculating the variation in pore volume and rocks [3].

There are some ways to carry out tests to determine the compressibility of rocks according to the literature. The most common determines the compressibility of the pore volume by applying hydrostatic stress and constant pore pressure (usually ambient pressure) [4].

In addition, some authors propose a little more sophistication when carrying out tests under hydrostatic confining pressure. In some cases, tests are performed by applying pore pressure at a certain level and in a controlled manner varying the initial pore pressure until lower pore pressure levels are reached, simulating reservoir production. Recently, a methodology was standardized by the International Society of Rock Mechanics (ISRM) [5].

Another option for obtaining elastic parameters is to carry out dynamic (acoustic) tests, in the case of rocks, which directly measure the velocities of elastic waves in the sample and use these velocities to estimate the elastic parameters based on the wave equation.

With the aim of obtaining correlations between static and dynamic elastic moduli, a wide variety of experiments have been carried out and, generally, the elastic moduli are obtained from stress and deformation measurements acquired through mechanical tests on rocks (“static modulus”) are different from the results for the same parameters obtained by carrying out acoustic tests (“dynamic module”) [1]. Such relations are relevant because there is a lot of data on wave propagation velocities, whether obtained from laboratory tests or sonic well recordings, for example.

### Theoretical Basis

### Linear Elasticity and Poroelasticity

The ability of a body to resist and recover from deformations produced when subjected to some type of stress is called elasticity. Basically, the theory of elasticity seeks to understand and quantify the behavior of materials (stresses and deformations) when subjected to some type of stress.

To understand the behavior of rocks, especially those linked to the oil industry, the concepts of elasticity are not sufficient and need to be better explored, as they are materials with relevant values of porosity and permeability. For this understanding, a step further is necessary regarding the presence of pores in the material. This consideration is made especially using the theory of poroelasticity.

It is important to highlight that rocks in general have more complex mechanical behavior, that is, simply addressing to the response of rocks within the elastic regime is not sufficient to predict their response when subjected to a variation in their stress state. The most correct approach is to use elastic-plastic models to represent this behavior as realistically as possible [7]. However, for the purposes of the present invention, it will be considered that, in the proposed methodology, the samples will preferably be in the elastic regime.

For a better understanding of the present invention, the most relevant theoretical foundations will be reviewed.

### Poroelasticity and Compressibility of the Pores

The theory of elasticity is crucial for understanding the behavior of materials, but it needs to be further refined to correctly understand the behavior of materials that have relevant porosity and permeability, such as rocks. To this end, poroelasticity evaluations were developed to consider the presence of pores in the materials.

In Geertsma [7], an attempt was made to present a unified treatment of the rock mechanics problems related to oil production engineering. The theory of poroelasticity was outlined and general solutions in terms of displacement functions were presented.

Zimmerman [8] went further and presented four compressibilities for porous rocks, relating changes in pore and rock volumes to changes in pore and confining pressures. Also using a micromechanical theory based on classical linear elasticity, three relations were established between these compressibilities. Wang [9] deepened the study and evolved the subject by applying it to Hydrogeology and Geomechanics situations.

As rocks are porous media that can be subjected to an external pressure (confining pressure) and a fluid pressure in the pores (pore pressure), there are several definitions of compressibilities associated with rocks.

Four compressibilities are defined [8], considering that there are two independent volumes (bulk and pore volumes) and that the two pressures (confining pressure and pore pressure) can vary. Two of these compressibilities are associated with pore volume.

These compressibilities may be related to deformations that occur in the rock volume (ε_{b}) or in the pore volume (εₚₒᵣₑ) defined in Equations (1) and (2).

$${ε}_{b} = \frac{d {V}_{b}}{{V}_{b}} \qquad ( 1 )$$

$${ε}_{p o r e} = \frac{d {V}_{pore}}{{V}_{pore}} \qquad ( 2 )$$

Where V_{b} represents the total volume and Vₚₒᵣₑ is the pore volume.

Further, according to Zimmerman [3], considering the confining pressures (P_{c}) and the pore pressures (Pₚ) acting on a rock in a hydrostatic regime, it is possible to relate the deformations in the rock with the pressure variations acting (Equation 3).

$$( \begin{matrix}{ε}_{b} \\ {ε}_{pore}\end{matrix} ) = [ \begin{matrix}- {C}_{bc} & {C}_{bp} \\ - {C}_{pc} & {C}_{pp}\end{matrix} ] \cdot ( \begin{matrix}{dP}_{c} \\ {dP}_{p}\end{matrix} ) \qquad ( 1 )$$

Or more directly:

$${ε}_{b} = - {C}_{bc} \cdot {dP}_{c} + {C}_{bp} \cdot dPp \qquad ( 2 )$$

$${ε}_{pore} = - {C}_{pc} \cdot {dP}_{c} + {C}_{pp} \cdot dPp \qquad ( 3 )$$

The compressibility matrix terms are defined as follows.

One of the compressibilities associated with the total volume (C_{bc}) is presented in Equation 6 (the other in Equation 8) and is the variation in the total volume depending on the variation in confining pressure (constant pore pressure):

$${C}_{bc} = - \frac{1}{{V}_{b}^{i}} {( \frac{\partial {V}_{b}}{\partial {P}_{c}} )}_{{P}_{p}} \qquad ( 4 )$$

The first sub-index indicates which volume the compressibility is associated with (in the case of total volume, b) and the second indicates which pressure changed to impose this volume variation, in case c, as it is the confining pressure P_{c} that varies. The right-hand derivative has the usual sense of differential calculus: the change in apparent volume related to confining pressure for constant pore pressure. This parameter is the inverse of the volumetric deformation modulus or total modulus (K), according to Equation 7 [10].

$${C}_{bc} = \frac{1}{K} \qquad ( 5 )$$

The total modulus, also known as volumetric deformation modulus, can be defined as the ratio of the relative hydrostatic stress to the volumetric deformation of a sample [11].

The variation in total volume due to variation in pore pressure (constant confining pressure) is defined in Equation 8. It reflects the influence of the pore pressure on the total volume, being a reason used to study problems related to subsidence [3].

$${C}_{bp} = \frac{1}{{V}_{b}^{i}} {( \frac{\partial {V}_{b}}{\partial {P}_{p}} )}_{{P}_{c}} \qquad ( 6 )$$

The compressibilities associated with the pore volume are presented in Equations 9 and 10. The variation in pore volume as a function of the variation in confining pressure (constant pore pressure) is defined in Equation 9. And this compressibility is relevant for in-situ determination of the pore volume [3].

$${C}_{pc} = - \frac{1}{{V}_{pore}^{i}} {( \frac{\partial {V}_{pore}}{\partial {P}_{c}} )}_{{P}_{p}} \qquad ( 7 )$$

The change in pore volume due to changes in pore pressure while maintaining confining pressure constant emulates a process analogous to the primary production, and is commonly applied to issues related to reservoir evaluation and is defined in Equation 10, according to Zimmerman [8]:

$${C}_{pp} = - \frac{1}{{V}_{pore}^{i}} {( \frac{\partial {V}_{pore}}{\partial {P}_{p}} )}_{{P}_{c}} \qquad ( 8 )$$

The change in pore volume in relation to the change in pore pressure under a constant confining pressure reflects how much excess pore pressure can be stored in the pore space due to the increased pore pressure. This quantity is associated with the compressibility of the reservoir fluid in the basic equations used in oil reservoir simulators [3].

Usually, laboratory data are acquired under hydrostatic stress conditions and can be transformed into uniaxial compressibilities with the approximate relation presented in Equation 11 assuming valid linear poroelasticity and adopting a Biot coefficient α (Equation 12), a Poisson's ratio (v) specific to α∘ν reservoir and the grain compressibility (Cg).

$${C}_{pp , uni} \approx \frac{α}{3} ( \frac{1 + v}{1 - v} ) {C}_{pp} \qquad ( 9 )$$

$$α = 1 - \frac{{c}_{g}}{{c}_{bc}} \qquad ( 10 )$$

It should be noted that the scope of the present invention does not address to determining the correct relation that must be used to transform the result obtained in the hydrostatic condition to the uniaxial condition.

Occasionally, Cₚₚ is called “effective pore compressibility” and CPC “compressibility coefficient” or “consolidation coefficient” [10]. Another aspect that must be highlighted is the relation between the compressibilities C_{bc}, C_{bp}, C_{pc} and Cₚₚ. The compressibilities C_{pc} and Cₚₚ are related to each other (Equation 13) and depend on the compressibility of the grains (C_{g}) that form the rock [8].

$${C}_{pp} = {C}_{pc} - {C}_{g} \qquad ( 11 )$$

Analogously, the compressibilities C_{bc} and C_{bp} are related as shown in Equation 14 [8].

$${C}_{bp} = {C}_{bc} - {C}_{g} \qquad ( 12 )$$

Furthermore, using the rock porosity (ϕ), it is possible to correlate C_{bp} and C_{pc}(Equation 15) [8].

$${C}_{bp} = ϕ {C}_{pc} \qquad ( 13 )$$

Rearranging Equations 13, 14 and 15 results in the relation shown in Equation 16.

$${C}_{pp} = [ {C}_{bc} - ( 1 + ϕ ) {C}_{g} ] / ϕ \qquad ( 14 )$$

These presented coefficients are dependent on each other and there are only 2 independent variables, from which all others can be rewritten [8].

Another relevant parameter that must be measured is the Skempton coefficient B (Equation 17), which is defined as the ratio between the variation in pore pressure (Pₚ) due to the variation in isotropic stress (α) applied to a sample in a test in undrained conditions [9].

$$B = \frac{Δ {P}_{p}}{Δσ} \qquad ( 15 )$$

To estimate the Skempton values of a given saturated sample, Equation 18 can be used [12]:

$$\frac{1}{B} = 1 + \frac{∅ . K}{α} \cdot ( \frac{1}{{K}_{fl}} - \frac{1}{{K}_{g}} ) \qquad ( 16 )$$

Where K_{fl} is the volumetric modulus of the pore fluid and K_{g} is the volumetric modulus of the rock component.

Knowing the Skempton coefficient, it is possible to obtain the C_{pc} value with Equation 19 [12].

$${C}_{pc} = \frac{B}{( 1 - B )} \cdot ( \frac{1}{{K}_{fl}} - \frac{1}{{K}_{g}} ) \qquad ( 17 )$$

### Relevant Compressibility Parameters in Oil Reservoirs

The effects of compaction of the oil reservoirs due to oil extraction activities can act as a key mechanism in production, in the same way that water is expelled when squeezing a sponge. Furthermore, stress variations associated with decreased pore pressure also affect porosity and permeability, which will have effects on oil recovery [1].

For example, as a result of reducing pore pressure (ΔPₚ), the volume of oil produced (ΔV_{prod})—under reservoir conditions—can be described in Equation 20 [1].

$$Δ {V}_{prod} = - {V}_{pore} . ( {C}_{f} + {C}_{pp} ) Δ {P}_{p} \qquad ( 18 )$$

Where C_{f} represents the compressibility of the fluid in the porous medium, which is equal to the inverse of the volumetric compression modulus of the fluid (C_{f}=1/K_{fl}).

The relation described above between volume variation and pore pressure variation illustrates that, for a rock with low rigidity and, therefore, with high pore compressibility, the production will be increased by pore compaction. This case is the so-called compaction “drive”, as it is mainly responsible for the production of crude oil [1].

Analogously to the previous one, in the case of high rigidity rock (low compressibility), the production of the drive will be determined by the compressibility of the fluid.

To evaluate the effects of stress variation on porosity, it can be remembered that the Biot coefficient (Equation 21) “is a measure of change in pore volume relative to the change in total volume at constant pore pressure”

$$α = ϕ \frac{Δ {V}_{pore} / {V}_{pore}}{Δ {V}_{b} / {V}_{b}} = \frac{Δ {V}_{pore}}{Δ {V}_{b}} \qquad ( 19 )$$

Thus, it is possible to estimate porosity changes by measuring the total volume or variation in pore volume (Equation 22).

$$\frac{Δϕ}{ϕ} = ( 1 - \frac{ϕ}{α} ) \frac{Δ {V}_{pore}}{{V}_{pore}} \qquad ( 20 )$$

Such relations can be verified in more detail in the work of Zimmerman [13], where they are also presented for the test situation with uniaxial deformation. An interesting application of this influence is found in a paper published by Alam et al. [14].

The production rate of a reservoir due to a certain amount of depletion is largely controlled by its permeability. There is also a consequent reduction in the permeability of the reservoir [1], [15] with the variation in stress resulting from oil production, thus showing an increase in effective stress.

### Determination of the Poroelastic Parameters

Poroelastic parameters can be determined in several ways, depending on the level of information required and the availability of suitable samples for laboratory testing. Some methods are based on relations derived from tests in different lithologies, but not necessarily in the field of interest. There are many methods published in the literature, and recently a methodology was standardized by the International Society of Rock Mechanics (ISRM) [2].

Some static experimental methods are used to understand the different compressibilities of rocks in the laboratory and, to obtain the results, it is necessary to expose a sample to confinement and pore pressure, in a controlled way, and then calculate the variation in pore and rock volume [3].

There are some ways in the literature to carry out static tests to determine the compressibility of rocks. The most common determines the compressibility of the pore volume by applying hydrostatic stress and constant pore pressure (generally atmospheric) [4].

In addition, some authors propose a little more sophistication when carrying out tests with hydrostatic pressure. However, they apply pore pressure at a certain level and in a controlled manner, varying the initial pore pressure until lower pore pressure levels are reached, simulating in some way the production of reservoirs.

To establish a guideline for the tests to be carried out, ISRM proposed a methodology to measure the compressibilities of interest and reproducing the most common reservoir conditions in the oil industry. At the same time, it emphasizes uniaxial compression testing [5]. The two testing protocols suggested by ISRM are briefly described below:

a) Cₚₚ (“Constant Pore Pressure”)—Uniaxial deformation with constant pore pressure
Axial stress as control parameter (“delta-stress” test).
Simulates the initial situation of oil pre-production (in this case, the stress must reproduce the effective in-situ stress—the pore pressure reaches a small value).
Simulates vertical compaction with restricted lateral stress.
This protocol directly measures the values of C_{bc,uni} and C_{pc,uni}.

Some other compressibility parameters can be obtained using the two above-measured parameters as input data, according to the given relations.

b) PpD (“Pore pressure Depletion”)—Uniaxial deformation with variation in pore pressure:
Pore pressure as a control parameter (“delta pore pressure” test).
Simulates the initial oil pre-production situation (the test must reproduce the in-situ stress).
By varying the pore pressure, the axial deformations are measured simulating vertical compaction.
This protocol directly measures the values of C_{bp,uni} and C_{pp,uni}.

The other compressibility parameters will be obtained using the two parameters measured above as input data, according to the relations presented previously.

Another way to obtain compressibility results is by using some established empirical methods developed by Newman [16] and Hall [17].

Newman correlations were established for consolidated sandstones (0.02<ϕ<0.23) and are indicated in Equation 23.

$${C}_{pp} = \frac{97 , {3.1}^{- 6}}{{[ 1 + 55 , 9. ϕ ]}^{1 , 429}} \qquad ( 21 )$$

Furthermore, Newman obtained correlations for carbonates (0.02<ϕ<0.33) and is presented in Equation 24.

$${C}_{pp} = \frac{0 , 856}{{[ 1 + 2 , {48.1}^{6} ϕ ]}^{0 , 930}} \qquad ( 22 )$$

Where Cₚₚ is given in psi⁻¹ (1 psi=6.895 kPa).

Newman correlations were obtained through laboratory tests. They included direct measurements of fluids expelled from samples by the application of stresses and inferences about the alteration in pore volume from changes in the sample dimensions as a function of applied stresses [16].

Pore variation measurements were obtained on stress paths with effective stress increments of 500 psi (3.447 MPa) up to the estimated in-situ lithostatic pressure applied to the sample [16].

Hall correlations have been established for limestone and sandstone [17] and are presented in Equation 25.

$${C}_{pp} = \frac{1 , 782}{{ϕ}^{0 , 438}} \qquad ( 23 )$$

Where Cₚₚ is given in psi⁻¹ (1 psi=6.895 kPa).

The measurements taken to achieve this correlation were performed with the maximum confining pressure limit at 3000 psi (20.684 MPa) and pore pressure variation from 0 to 1500 psi (10.342 MPa) [17]. It is important to highlight that the recommendation is to carry out compressibility measurements of the rock formation, and the correlations should only be used to estimate their magnitude [18].

### Oil Reservoirs and Poroelastic Parameters

Obtaining correct poroelastic parameters for studies involving reservoirs applies to the following evaluations: in flow and geomechanical simulators and in monitoring reservoir behavior throughout production (application of 4D seismic).

### Numerical Simulation of Oil Flow in Reservoirs

In the numerical simulation of flow in reservoirs, Rosa et al. [19] highlight, as illustrated in FIG. 2.1, that the mathematical model uses various geological information, such as characteristics of fluids and rocks, production history, among others.

The steps normally followed in carrying out a reservoir study, using numerical simulators, can be summarized in FIG. 2.2 [19].

As briefly described previously and now expanding to the specific case of oil reservoirs, the flow of fluids in porous media demands not only fluid parameters, but also those related to the mechanical behavior of the rock [20]. For example: when pore pressure (Pₚ) varies due to oil production—remembering Terzaghi's principle of effective stress (Equation 25)—the stress (σ_{eff}) in the reservoir rocks also varies.

$${σ}_{eff} = σ - {P}_{p} \qquad ( 24 )$$

This change in stress in the reservoir causes deformations in the rock that, in turn, can alter the porosity and, consequently, its permeability. This last condition has a direct impact on the exploration of the field, that is, on the pore pressure of the reservoir. In addition, the deformation of the rock itself can influence the pore pressure, increasing it by reducing the pore space and the mechanical parameter that represents this change is the compressibility of the rock pores [20].

Once again, it should be emphasized that improving the determination of poroelastic parameters can bring gains to reservoir management, as well as to the development of simulators with an integrated solution [21].

4D Seismic Monitoring and Integration with Geomechanics

One of the most important steps in exploring for oil present in reservoir rocks is seismic research to obtain data such as the depth of the rocks, differences between the different rock layers present at the site and the indication of the presence of oil and gas.

After the first seismic survey and already in the production phase, new surveys are carried out to obtain evidence of changes in fluids and rocks. This survey carried out at separate times is known as 4D seismic.

4D seismic monitoring is a technique with potential to support the management of oil fields, aiming at increasing production and the recovery factor, as well as guarantee the safety of operations. Time-lapse (4D) seismic interpretation is based on the differences in amplitude, impedance and time shift between the Base and Monitor volumes.

These differences are mainly due to the variation in seismic wave velocity across geological layers induced by production, mainly related to fluid saturation and pore pressure [22].

The mechanisms that affect time changes are separated into three main categories [23]:
Production-induced deformations in overload and underload.
Change in pore pressure and internal deformation of the reservoir.
Variation in the fluid saturation during production.

Equation 27 shows the correlation between time displacements and vertical deformations in a reservoir

$$\frac{Δ t}{t} = ( 1 + R ) . {ε}_{zz} \qquad ( 25 )$$

Where:

$$\frac{Δ t}{t} - variation in time shift between seismic measurements carried out at separate times ( geophysical magnitude ) - monitor \times baseline$$

ε_{ZZ}—vertical deformation of the reservoir
R—factor that correlates

$$\frac{Δ t}{t}$$

### to ε_{ZZ}.

To estimate the vertical deformation of the reservoir, the relation between the deformation and the pore pressure variation can be used, the uniaxial compressibility of the rock pores being known (Equation 28). Therefore, it is another relevant application to know the appropriate value of the Cₚₚ parameter of oil reservoir rocks.

$${ε}_{zz} = - {C}_{pp , uni} \cdot {dP}_{p} \qquad ( 26 )$$

### Rock Physics

This section will briefly present some relevant topics associated with the propagation of elastic waves in isotropic media, as well as the relations and models that estimate elastic parameters from measurements of the wave propagation velocity in dry and saturated rocks.

### Propagation of Elastic Waves in Rocks

Elastic waves are mechanical disturbances that propagate through a material. When this occurs through air or water, the waves are called acoustic. This last name is also used to replace the term elastic waves in rocks [1].

FIG. 2.4 specifically indicates how acoustic waves propagate in a rock. The so-called P (primary) wave is a longitudinal wave or compression wave. The so-called S (secondary) wave is a transverse wave or shear wave.

Measuring the propagation velocity of P and S waves in different ways and from different perspectives is quite common in the oil industry to meet different needs, from geological evaluations of inaccessible formations to the case of determining elastic parameters by means of laboratory measurements.

In an elastic and isotropic material, once some of its elastic parameters—K, G and ρ—are known, it is possible to determine the values of the compression wave velocities (Vₚ) and the shear wave velocities (V_{S}).

For compression waves, the relation is presented in Equation 29 [25]. And for shear waves, Equation 30 defines the relation [25].

$${V}_{P} = \sqrt{\frac{K + \frac{3}{4} G}{ρ}} \qquad ( 27 )$$

$${V}_{S} = \sqrt{\frac{G}{ρ}} \qquad ( 28 )$$

Where G is the shear modulus.

When the wave velocity values and total rock density are known, the dynamic elastic parameters can be calculated from Equations 31, 32 and 33.

$$E = ρ . {V}_{S}^{2} . \frac{3. {V}_{P}^{2} - 4. {V}_{S}^{2}}{( {V}_{P}^{2} - {V}_{S}^{2} )} \qquad ( 29 )$$

$$v = \frac{( {V}_{P}^{2} - 2. {V}_{S}^{2} )}{2. ( {V}_{P}^{2} - {V}_{S}^{2} )} \qquad ( 30 )$$

$$K = ρ . ( {V}_{P}^{2} - \frac{4}{3} {V}_{S}^{2} ) \qquad ( 31 )$$

Depending on the components of the rocks, the presence and type of fluids, state of stress and porosity, the following trends stand out for the propagation velocities of the elastic waves: the greater the elastic modulus of the rock minerals, the greater the wave velocities; the velocity of the compressive wave is dominated by the type of fluid present in the pores (see Equation 29) and the velocity of the shear wave is also influenced by the presence of fluids in the pores at a lower level, just because the density of the fluid changes (see Equation 30); the greater the stress, the greater the value of wave velocities, and the increase in porosity decreases the velocities of the compression and shear waves [25].

### Models for Elastic Properties

For a theoretical description, natural rock, as a heterogeneous system, with internal structure, must be idealized for the formulation of elastic properties of the rock, in terms of volume fractions and properties of the components (minerals and fluids), rock texture, pressure [26].

The elastic properties of a composite material, such as a sedimentary rock, depend mainly on the following factors [1]: the relative amount of each component present; the elastic properties of each component; the geometric distribution of each component and the porosity.

It is often not possible to obtain the factors described above precisely, making it necessary to accept relatively simpler models [1]. They can be classified according to the type of “geometric idealization” of the rock (single layer models, sphere models, inclusion models) [25]. The limits of the elastic moduli are established according to the different models.

The upper and lower limits of the elastic parameters of a porous medium can even be described as illustrated in FIG. 2.4 [26]. They are used, for example, to verify the modulus of elasticity calculated by the Vₚ/Vₛ relations measured in the laboratory [26].

Knowing the mineral components of the rock, it is possible to calculate the values of the volumetric compressibility modulus of the matrix with the three relations, one defined as the Voigt average (K_{V} —Equation 34), the other defined as the Reuss average (K_{R}— Equation 35) and the last known as the Voigt-Reuss-Hill average (K_{VRH}—Equation 36).

$${K}_{V} = {\sum }_{i = 1}^{n} {k}_{i} . {V}_{i} \qquad ( 32 )$$

$${K}_{R} = {[ {\sum }_{i = 1}^{n} \frac{{k}_{i}}{{V}_{i}} ]}^{- 1} \qquad ( 33 )$$

$${K}_{V R H} = \frac{{K}_{V} + {K}_{R}}{2} \qquad ( 34 )$$

To estimate the effective modulus of elasticity of the grain (K_{g}) of each sample in this work, the Voigt-Reuss-Hill average was used (K_{VRH} —Equation 36), as it comprises the Voigt average (represents the upper limit of the value K) and the Reuss average (represents the lower limit of the value K).

### Gassmann Model for Estimating Elastic Properties of Saturated Rocks

The Gassmann model [27] can be used to estimate the elastic properties of porous rocks, allowing the prediction of velocities of rocks saturated with a single fluid (e.g., water) from velocities of rocks saturated with another fluid (e.g., gas) and vice versa.

Gassmann's theory assumes the following hypotheses [28]:
The rock is macroscopically homogeneous and isotropic: this assumption guarantees that the wavelength is greater than the grain size and pore size.
Within the interconnected pores, there is fluid pressure balance and there is no pore pressure gradient due to the wave propagation.
The pores are filled with non-viscous, frictionless fluids. This also contributes to pore pressure balance and results in a shear modulus that is independent of the porous rock fluid.
The rock-fluid system is closed (not drained).
Pore fluid does not interact with solid material or rock structure.
The passage of the waves results in movement (displacement) of the entire rock section and there is no relative movement between fluid and rock skeleton.

The alteration in pore fluid influences the elastic wave velocity due to the elastic modulus and the alteration in density. The effects are presented below:

1. Rock density follows Equation 37.

$$ρ = ( 1 - ϕ ) . {ρ}_{g} + ϕ . {ρ}_{f l} \qquad ( 35 )$$

2. The shear modulus is independent of the type of fluid.

3. The volumetric modulus of compression (K) is strongly dependent on the compressibility of the fluid. The K parameter is the main one in Gassmann models.

The left side of FIG. 2.5 shows the dry case, where the pores are empty. The pore fluid has a zero mass modulus and does not contribute to compressive stiffness. The deformation behavior is characterized by the moduli of the two systems or rock skeleton—volumetric deformation modulus of dry rock (K_{dry}) and shear modulus of dry rock (μ_{dry}).

The right side of FIG. 2.5 describes the “fluid saturated” case. The deformation behavior is characterized by two moduli:
a) effective volumetric modulus for saturated rock—Kₛₐₜ>K_{dry}.
b) effective shear modulus for dry rock is equal to the modulus for saturated rock—μ_{dry}=μₛₐₜ (shear modulus for saturated rock).

The effective volumetric modulus of deformation of the saturated rock results from the combination of deformation of the rock skeleton, solid components and fluid.

Equations 38 and 39 demonstrate the relation

$${K}_{sat} = {K}_{d r y} + \frac{{( 1 - \frac{{K}_{d r y}}{{K}_{g}} )}^{2}}{\frac{ϕ}{{K}_{f l}} + \frac{1 - ϕ}{{K}_{g}} - \frac{{K}_{d r y}}{{K}_{g}^{2}}} \qquad ( 36 )$$

$$\frac{{K}_{d r y}}{{K}_{g} - {K}_{d r y}} = \frac{{K}_{s a t}}{{K}_{g} - {K}_{s a t}} - \frac{{K}_{f l}}{ϕ . ( {K}_{g} - {K}_{f l} )} \qquad ( 37 )$$

The main process for replacing type 1 fluid with type 2 fluid is shown in FIG. 2.6 [25].

Correlations of Results with Experiments Using Static and Dynamic Tests

It is possible to estimate values for elastic parameters of any material (section of Propagation of elastic waves in rocks) using the value of apparent density (φ, compression wave velocity (V_{P}) and shear wave velocity (V_{S}). To corroborate these relations, experiments were carried out with a 316LN austenitic steel [29], in which material samples were tested using static and dynamic methods (high vibration frequencies).

As a result, it was demonstrated that for the material in question—which is a homogeneous and elastic material—the Young's moduli values obtained by the two methods are very close, since “Thermodynamics predicts that the real differences are below the detection by usual measurements” [29], corroborating what is expected about the results obtained from the same module using the correlations of the elastic wave equation.

It should be highlighted that this used material is homogeneous and presents linear elastic behavior when subjected to stresses below certain limits. In the case of rocks, it can be assumed that it is an easy task to obtain the elastic moduli of a rock simply using these acoustic test correlations.

However, there is a wide variety of experiments that indicate that the elastic moduli obtained from stress and deformation measurements acquired through static tests on rocks (“static moduli”) are sometimes significantly different from the results obtained for the same parameters obtained through acoustic tests (“dynamic moduli”) [1].

In general, the modulus values derived from dynamic tests are greater than those obtained from static tests.

The biggest difference between static and dynamic measurements is the deformation amplitude, not the deformation rate. The static modulus, measured from the slope of the stress-deformation graphs, differs from the dynamic modulus, with a small deformation amplitude, due to the plasticity and non-linear effects [1].

One of the causes of the discrepancy between the values obtained by “static” and “dynamic” tests is related to the heterogeneous microstructure of the rocks. In addition, a large part of this effect is expected to originate from grain contacts, as stress concentrations in contact areas exceed the yield point of the material even at low stress levels [1].

The loading and unloading cycles in a static test, as illustrated in FIG. 2.7, represent in some way an intermediate stage between a standard static test and a dynamic test, as the material will experience stress cycles (when applying a smaller amplitude of loading and unloading cycle—see the arrows in FIG. 2.7) similar to the elastic wave propagation in a dynamic test [1].

There are several works reporting research to compare elastic parameters obtained by static and dynamic methods.

Simmons et al. [30] work with results in elastic parameters obtained by static and dynamic methods performed on varied materials (granite, quartzite, limestone, aluminum, diabase) and using different levels of confining pressures.

Regarding the results, the following conclusions stand out: for metals, the agreement of values calculated from different methods is remarkably high, as would be expected, since these materials can be modeled as exhibiting elastic behavior. For other materials (rocks) the results do not show a good correlation, as explained below:
The moduli obtained by the “static” methods are generally of lower value than those achieved by “dynamic” methods.
For higher confining pressures, the differences in the values of these moduli decrease, although they are still different. This situation arises from the fact that any discontinuities in the sample “close” during the process of increasing pressure, leading the material to a more “elastic” behavior [30].

Briefly, King [31] remembered that there are two methods indicated as possible to be used to determine elastic parameters:
a) determination of the stress-deformation relations obtained in the laboratory—indicated as “static elastic constants”.
b) calculated from the velocities of the elastic waves measured in the rock, indicated as “dynamic elastic constants”.

Some authors have demonstrated theoretically that the differences between the results obtained by static and dynamic methods originated from the presence of discontinuities in the rocks, suggesting that small cracks, for example, close as the confining pressure increases and the results in static and dynamic elasticity parameters have been closer to each other [31].

Simultaneous static and dynamic tests were also carried out on the same sample during the same test using suitable equipment to prove such assertions [31]. The main conclusions of this study by these authors were the following:
The dynamic moduli of isotropic dry sandstone are greater than the static ones. The ratio between them decreases as the hydrostatic pressure to which the sample is subjected also increases. This situation is explained by the presence of randomly scattered cracks that close with increasing hydrostatic pressure.
The dynamic moduli of a transversely anisotropic dry sandstone are greater than the static ones. The relation between them decreases as the hydrostatic pressure to which the sample is subjected also increases. The difference between static and dynamic moduli is greater in the direction perpendicular to the bedding planes than in the parallel direction [31].

In 1981, Cheng et al. [32] obtained the bulk modulus by static and dynamic methods and studied the relation between these moduli in different ways for some confining stresses. Several types of rocks were evaluated in that work, for example: Berea sandstone, Navajo sandstone, Westerly granite, Bedford limestone, among others.

In the case of the volumetric modulus (K), there is a correlation between the results of the dynamic and static methods, with the dynamic results being greater than the static ones. It was also demonstrated that the values of the dynamic method approach those from the static method as the level of stress to which the samples are subjected increases, indicating that this situation results from the stimulation of the contact between cracks in the rocks [32].

Some other authors have also made efforts to study the relations between Young's moduli obtained by static and dynamic methods [33], [34].

In 1995, Plona & Cook [35] reaffirmed that the static elastic moduli are generally smaller than dynamic ones. Some of the reasons for such differences are related to the inelastic behavior of rocks, as well as the effects of deformation amplitudes.

In this work, there were presented simultaneous results (Uniaxial Compressive Strength—UCS—with V_{P} and Vₛ measurements) on rock samples (Castlegate sandstones) with varied stress cycles (loading—unloading) to improve the understanding of the relation between static and dynamic moduli of this type of material [35].

In conclusion, it was shown that the static Young's modulus, when defined in terms of small amplitudes, with small loading-unloading trajectories, presents results equivalent to the Young's modulus obtained by dynamic tests along the direction of stress application. FIG. 2.8 illustrates the assertion made by the authors [35].

Still within the scope of comparisons between moduli, Wang [9] highlighted that the differences between the values of the static and dynamic moduli can be explained by three main factors: deformation amplitude, pore shape and pore geometry.

Regarding the deformation amplitudes, it was stated that in static tests they are about 10⁻² and in dynamic tests about 10⁻⁶. Therefore, for typical reservoir rocks, where there are long pores and cracks at the grain contacts, the effect of larger deformation amplitudes (static tests) results in inelastic deformation, which implies that the rock has greater compressibility in this case. However, in the case of the dynamic tests, as they mobilize smaller deformations than static tests, their behavior is often predominant in the elastic regime, which results in lower compressibility [9].

Observations made by Fjaer [36] in the experiment that determined the strength of less competent sandstones based on sonic measurements, indicated that rock failures gradually occurred when stresses were applied, while the stresses and deformations were altered, and that this process of gradual failure played a key role in the differences between the static and dynamic methods.

Expanding and deepening the discussion, in 2013, Holt et al. [37] indicated that the inelastic contribution to the static deformation appears to be the main reason for the differences in moduli obtained by static and dynamic methods. Dynamic moduli are measured at a certain frequency (500 to 800 kHz) and static moduli represent the low-frequency behavior depending on the test loading rate, which points to the following conclusions:
Static moduli are measured in finite deformations, indicating that the samples incorporate inelastic (plastic) deformations.
Dynamic moduli have very small deformations (of the order of 10⁻⁶) and can therefore be considered to have purely elastic behavior.

The effects of finite deformations are considered in this paper as the main sources of the differences between static and dynamic moduli. The physical mechanisms that lead to this conclusion can be separated into two groups: plasticization or crushing at grain contact caused by high intergranular stresses and frictional sliding between uncemented grains. These effects occur in static tests, but not in dynamic tests [37].

Based on the results of experiments with natural and synthetic sandstones, and small spheres of unconsolidated glass and sand, also with analogous shale, the dynamic moduli found have, in most cases, greater values than the static moduli [37].

In the case of perfectly smooth spherical particles (glass spheres), the dynamic and static moduli were shown to be equal within the limits of the experimental precision. This situation is valid if the compaction behavior is controlled by normal forces. When the stress path is altered, for example to uniaxial loading—which now includes the presence of shear stresses—or when particle shapes or surfaces are not perfect, intergranular slipping (and possibly plasticization at the grain contact) can occur. [38].

In the same paper, Holt et al. [38] found that, in the case of sandstones, the static and dynamic moduli were the same only in the case of highly cemented sandstones in “virgin” conditions, that is, before any damage was caused to the sample. In most cases, natural sandstones were damaged, either by the sample extraction process or by the weathering suffered by the material.

According to Fjaer et al. [39], one of the main reasons for the variations between the values of the moduli found by the static and dynamic method is related to inelastic processes, mainly those referring to the friction between the grains arising from the displacement between them. Therefore, for modeling purposes, the numerical model was used with a grain pack model based on other works [40], [41] and [42].

The aforementioned work reaches the level of stress behavior in grain-to-grain contact, and indicates the theoretical distribution of the stresses in the contact before and after sliding between grains, together with the application of normal stress or shear stress.

The model used (with round grains) can indicate the relation between static and dynamic moduli during the unloading cycle due to the microstructure being well defined.

Another issue concerns the distribution of stresses at grain contacts, which can vary depending on the actual grains of a rock, which are typically not spherical. It is also worth mentioning that the model grains are not cemented; therefore, in the real case where the grains are cemented, the results are also discrepant in relation to the real sample [39].

Yan et al. [43] performed simultaneous tests (static and dynamic) on 70 Berea sandstone samples varying the confining pressures at 5, 10, 20, 30, 40 and 50 MPa to evaluate the correlations of volumetric moduli for evaluating oil reservoirs. It has been demonstrated that, with increasing pressures, the ratio between static skeletal stiffness and dynamic skeletal stiffness is closer to values on the order of 0.75, whereas at lower pressures this ratio is 0.4. In other words, the greater the pressure to which the sample is subjected, the closer the obtained values [43].

Bilal et al. [44] performed triaxial and dynamic tests on samples of Austin limestone and three different sandstones (including Castlegate sandstone), and compared the obtained results of Young's modulus and Poisson's ratio. Furthermore, a quadratic regression was applied to the data obtained to separate the linear elastic mechanisms from the nonlinear mechanisms of the tests. According to the author, this was the first time that an attempt was made to “isolate” the effect of each elastic nature on the test results.

More recently, Armaghani et al. [45] carried out a compilation of data from 108 case studies of correlations between UCS data obtained by static and dynamic means and developed simple, linear regression, non-linear multiple regressions, artificial neural network and hybrid models to propose data correlations of various lithologies.

Onalo et al. [46] went further and proposed a new model to estimate the static modulus of Young's modulus considering a lithology-porosity dependent parameter. The model was tested on a dataset compiled from different sources covering a wide range of lithologies. Therefore, the model can be widely applied in various geological settings.

The study demonstrates that the previous models for estimating static Young's modulus from dynamic modulus do not adequately capture the nonlinear relation between static and dynamic values [46]. So, they proposed models to more accurately represent the nonlinear relation.

The performance of the proposed model is compared to existing models from various sources, as illustrated in FIG. 2.9, which presents the statistical analyses carried out to present the model predictions, and, in FIG. 2.10, which shows the correlations between the static and dynamic Young's moduli [46].

Finally, to study correlations between results of static and dynamic compressibility measurements, De Oliveira et al. [47] carried out uniaxial compression tests and acoustic wave propagation subjected to the hydrostatic pressure variation. They pointed out that “the correlation between the two methods was economically important for the industry and serves as a basis for the calibration process of logging tools”.

### BRIEF DESCRIPTION

The present invention pertains to the field of modeling, simulation and evaluation of reservoirs and discloses preferred embodiments of a method for determining static and dynamic elastic and poroelastic parameters of rocks under hydrostatic confining pressure conditions, system for carrying out said method and computer-readable storage medium.

### BRIEF DESCRIPTION OF THE DRAWINGS

The patent or application file contains at least one drawing executed in color. Copies of this patent or patent application publication with color drawings will be provided by the Office upon request and payment of the necessary fee.

In order to complement the present description and obtain a better understanding of the features of the present invention, the following FIGS. are presented:

FIG. 1A—Schematic representation of the compaction and subsidence in an oil reservoir.

FIG. 1B—Schematic summary of the steps of the present invention.

FIG. 2A—Applications of numerical simulations of reservoirs (Reproduction from Rosa et al.).

FIG. 2B—Steps of a reservoir study using a numerical simulator (Reproduction from Rosa et al.).

FIG. 2C—Schematic representation of elastic waves P and S [25].

FIG. 2D—Limits of K values for each model (adapted from Schan).

FIG. 2E—Derivation of the Gassmann equation (reproduced from Schan).

FIG. 2F—Process of replacing type 1 fluid with type 2 fluid.

FIG. 2G—Loading-unloading cycles in a rock mechanical test [1].

FIG. 2H—Reproduction of the stress-deformation curves obtained by the dynamic method and the static method [36].

FIG. 2I—Correlations between static and dynamic Young's modulus [47].

FIG. 2J—Correlations between static (Es) and dynamic (Ed) Young's modulus [47].

FIG. 3A—Step 1—Loading and unloading pressure cycle in the dry test.

FIG. 3B—First step process—Dynamic test with dry sample.

FIG. 3C—Schematic graph with the variation in confining pressure imposed by the pump and the response of the pore pressure to variations in confining pressure.

FIG. 3D—Step 2—Loading and unloading pressure cycle in the saturated test.

FIG. 3E—Second step process—Dynamic test with saturated sample.

FIG. 3F—Loading/unloading pressure path for Cₚₚ tests (step 3).

FIG. 3G—Third step process—Static compressibility test with saturated sample.

FIG. 3H—Compression and shear waves. Blue waves represent the behavior of the system and in red the time shift of the waves with the inserted sample [50].

FIG. 3I—P waves measured at each 30° increment.

FIG. 3J—S1 waves measured with each 30° increment.

FIG. 3K—S2 waves measured at each 30° increment.

FIG. 3L—Pressure path—1 loading and unloading cycle—dry hydrostatic test.

FIG. 3M—Variation of compressive wave velocities (Vₚ) at each pressure level of the loading and unloading cycles for the sample CGS-103.

FIG. 3N—Variation of compressive wave velocities (Vₚ) at each pressure level of the loading and unloading cycles for the sample IL3-103.

FIG. 30—Applied confining pressure variation cycles and resulting pore pressure variation values for calculating the Skempton coefficient after the first flow of five pore volumes in the sample CGS-103.

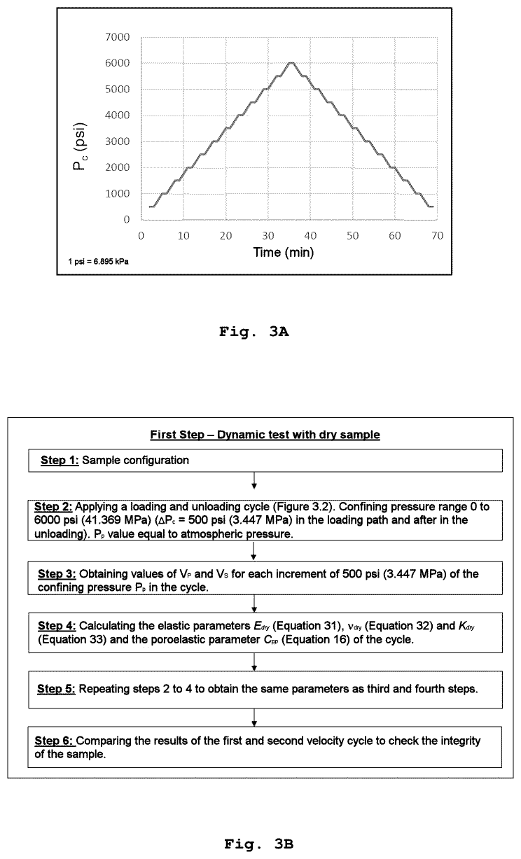

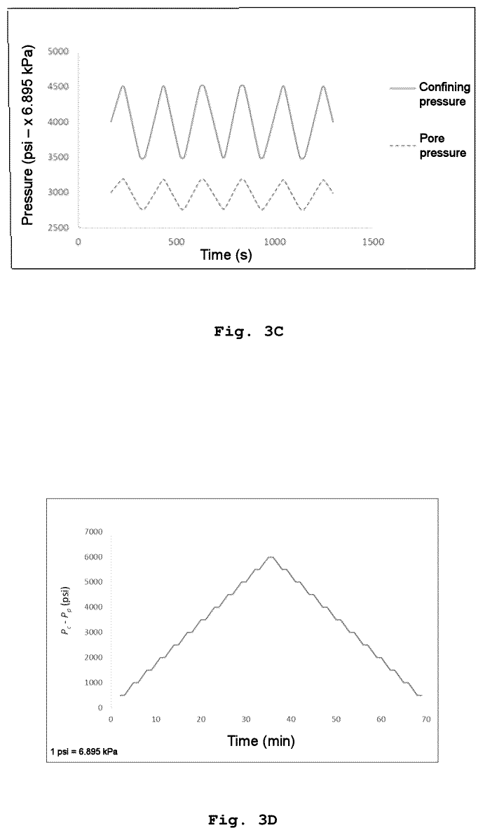

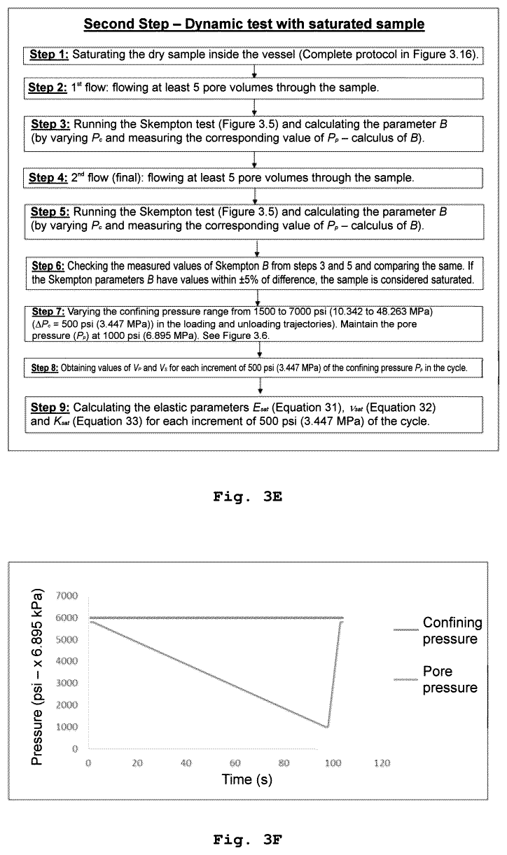

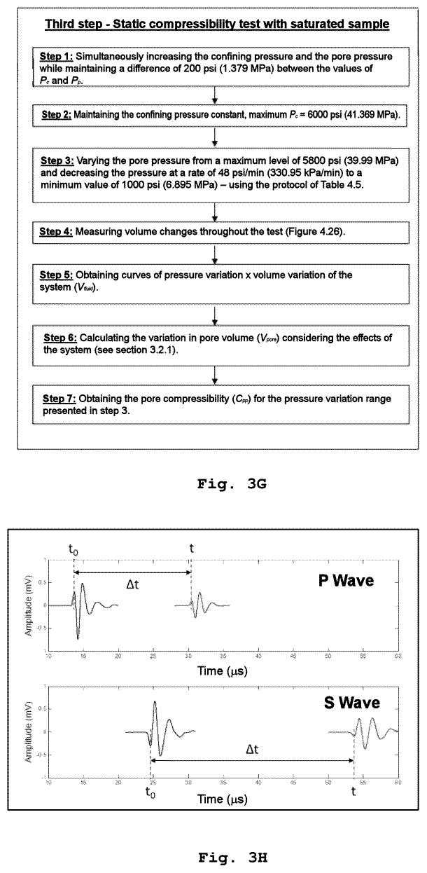

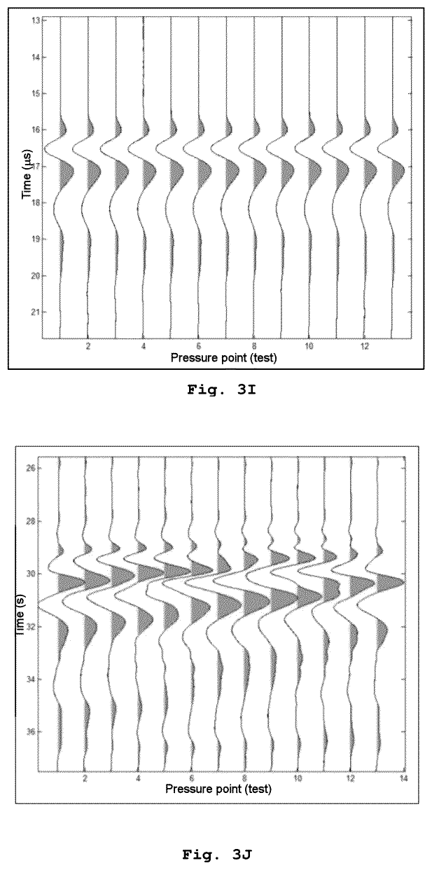

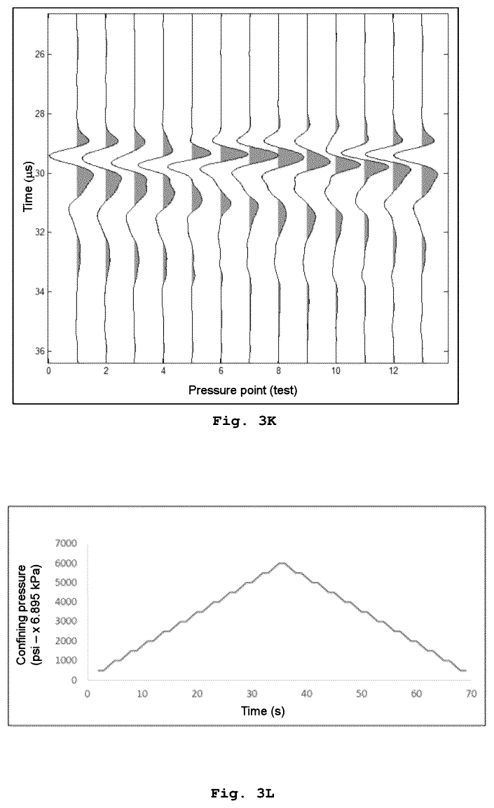

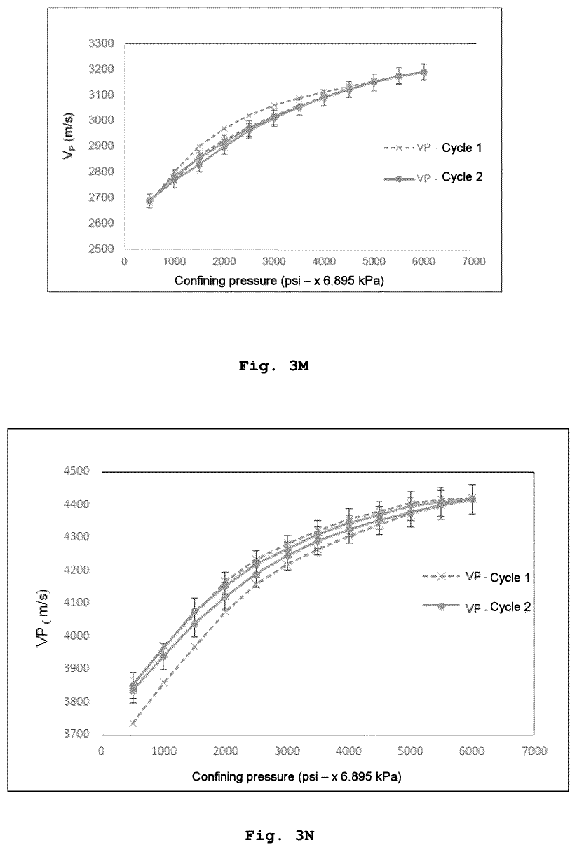

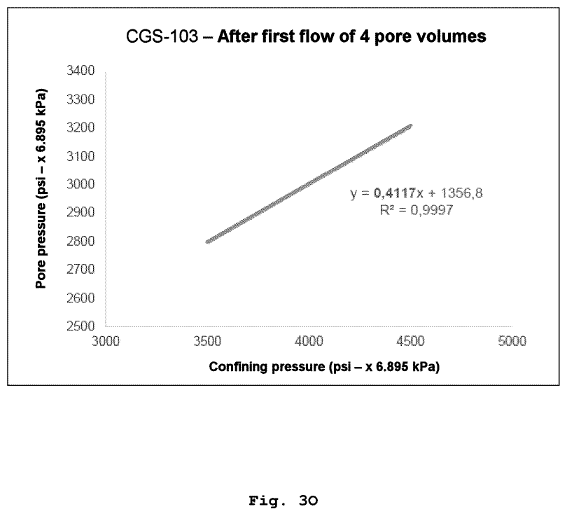

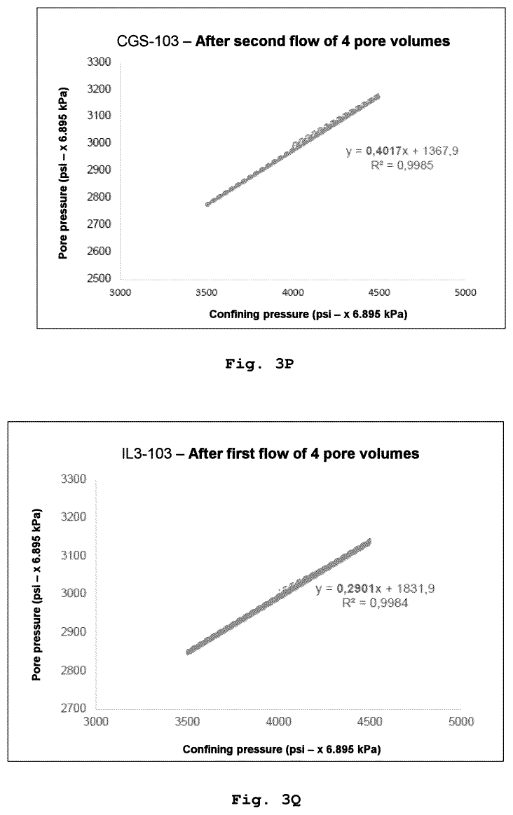

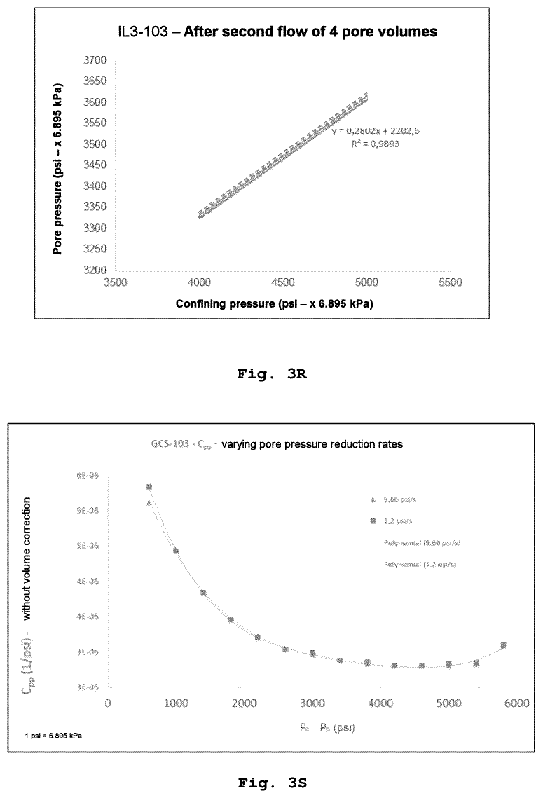

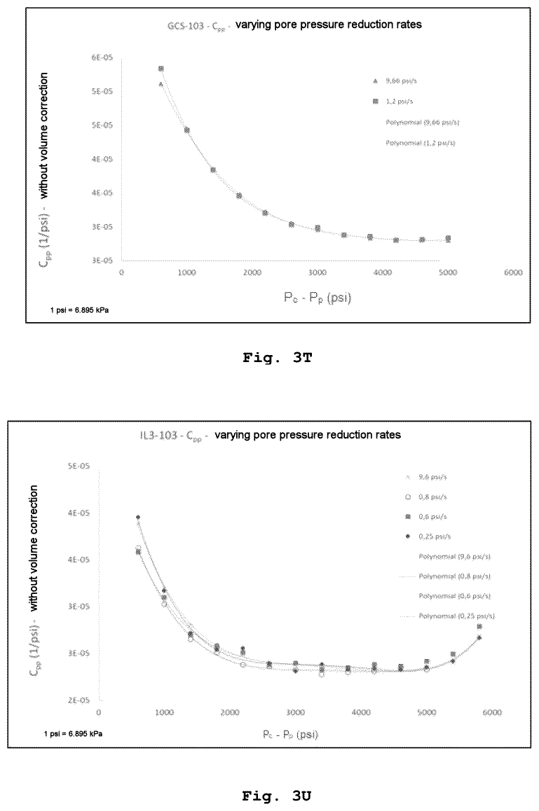

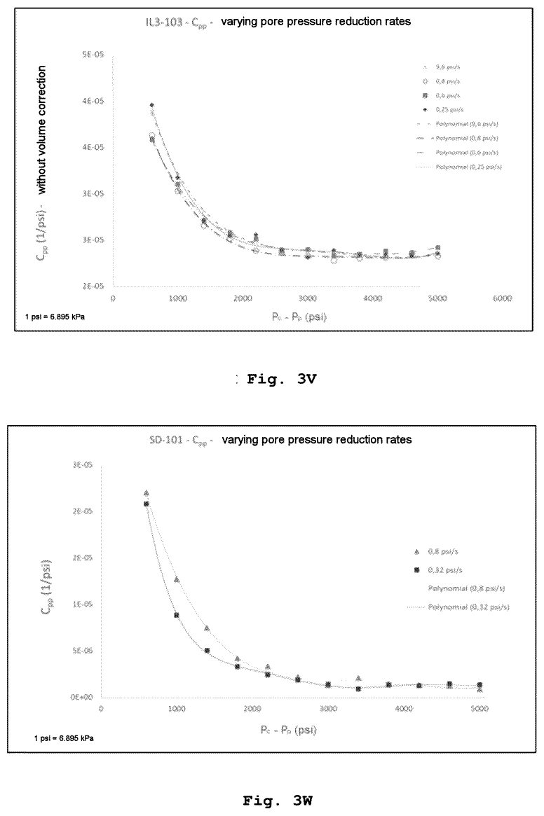

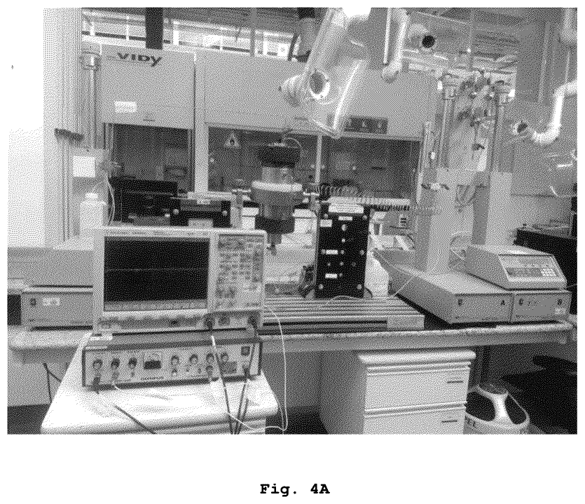

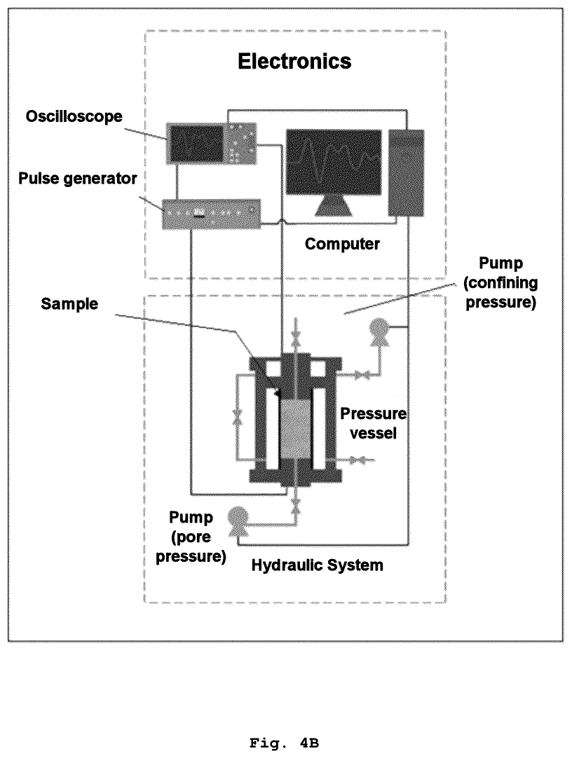

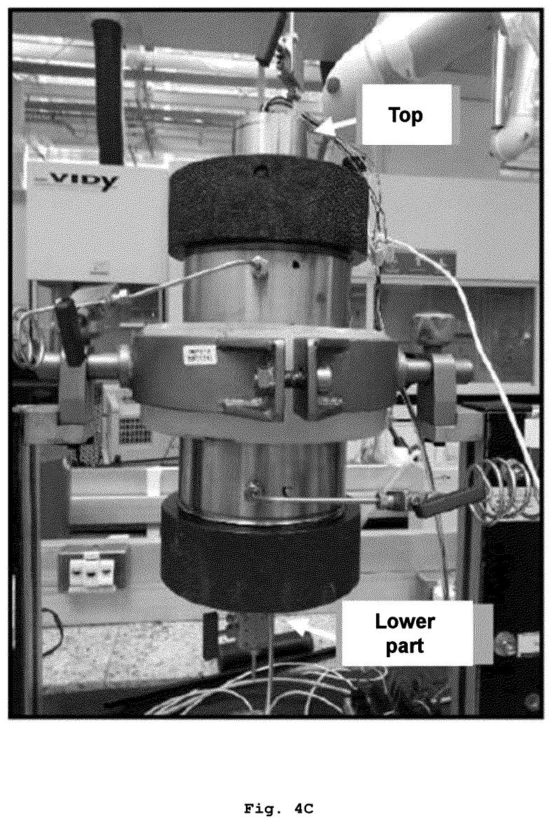

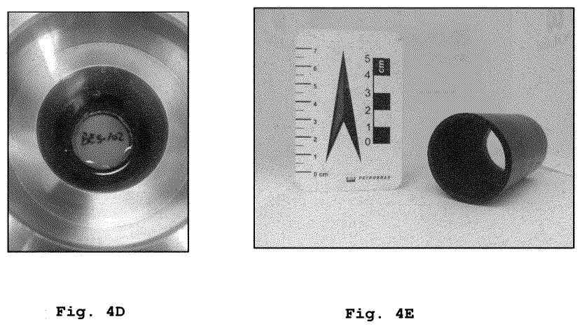

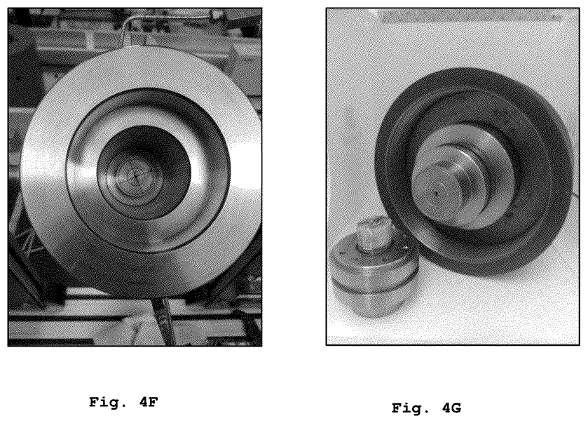

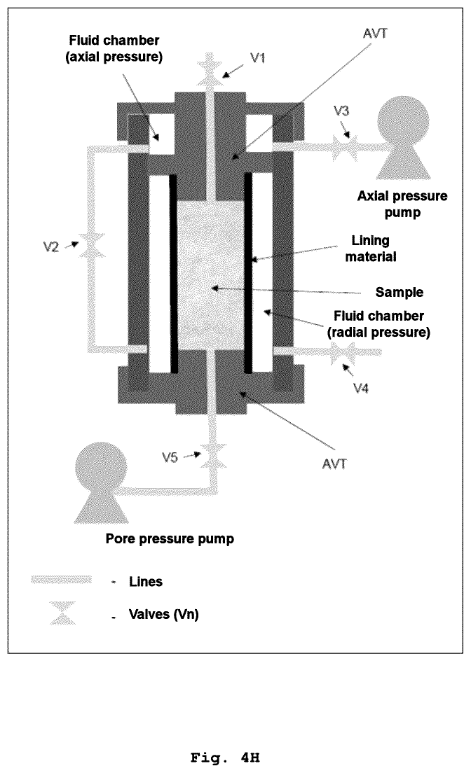

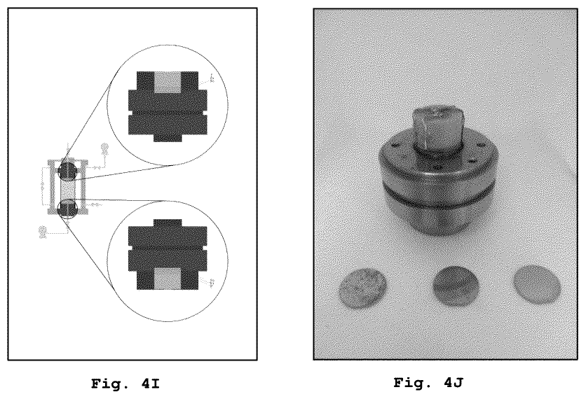

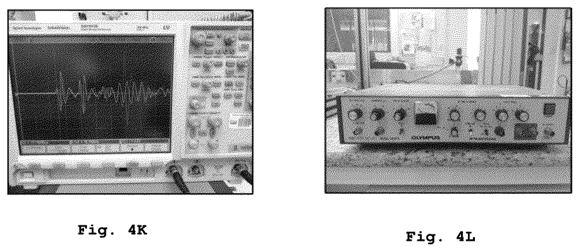

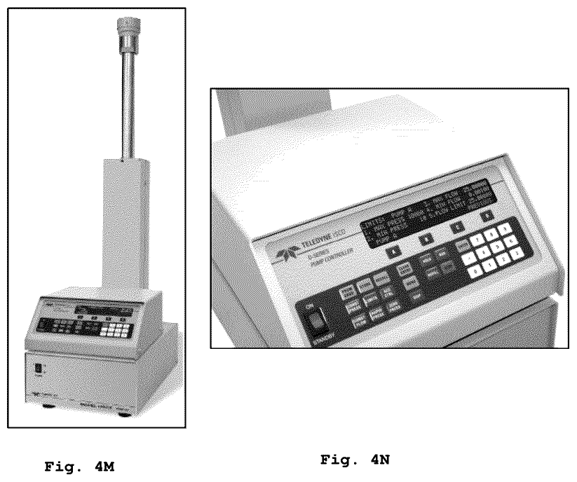

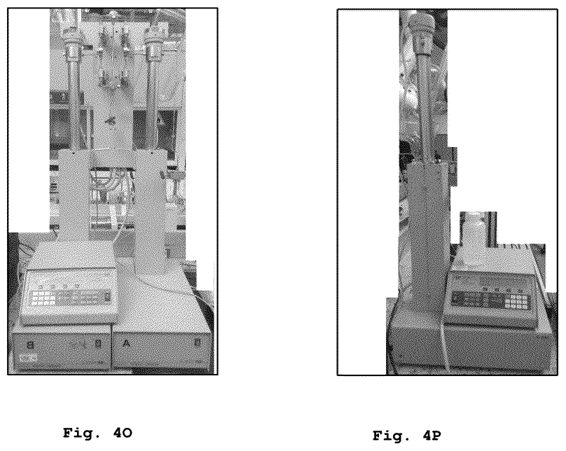

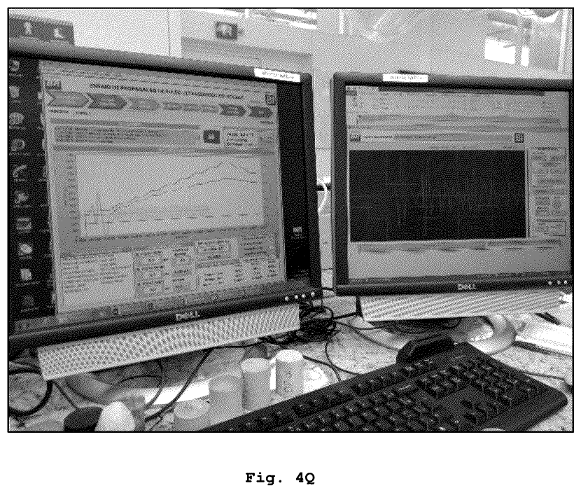

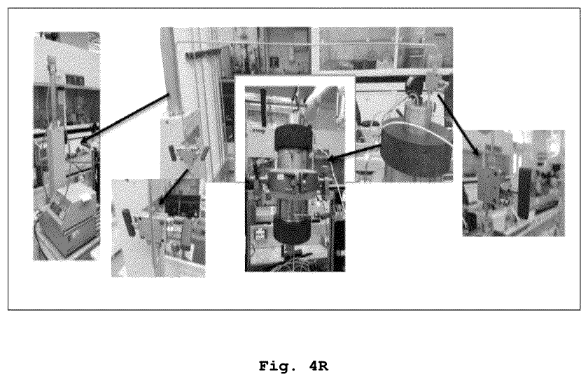

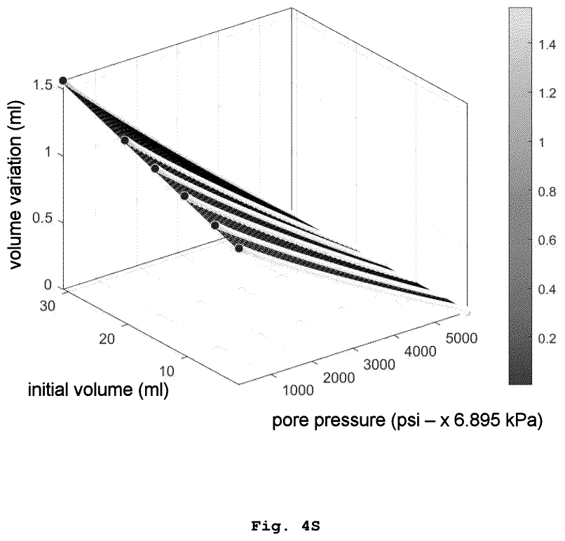

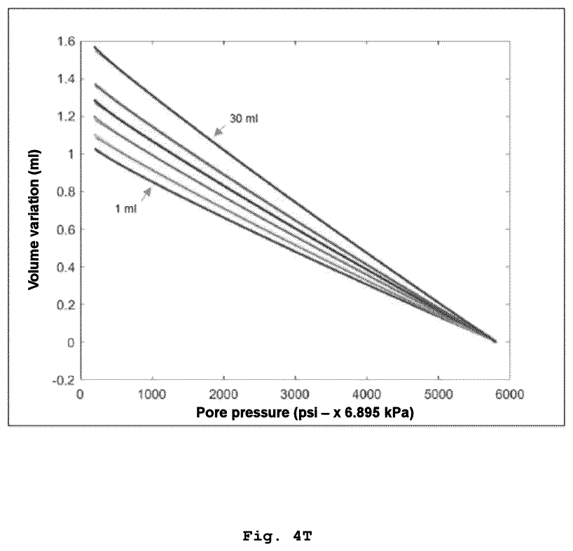

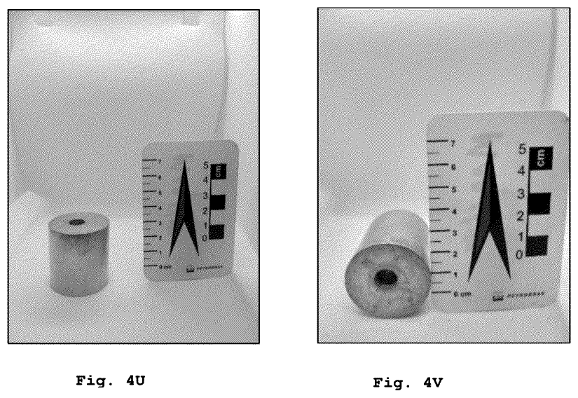

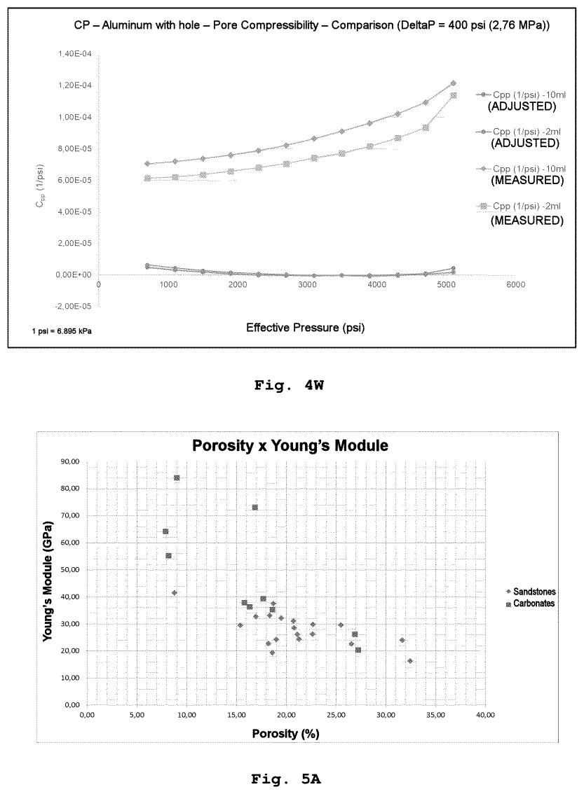

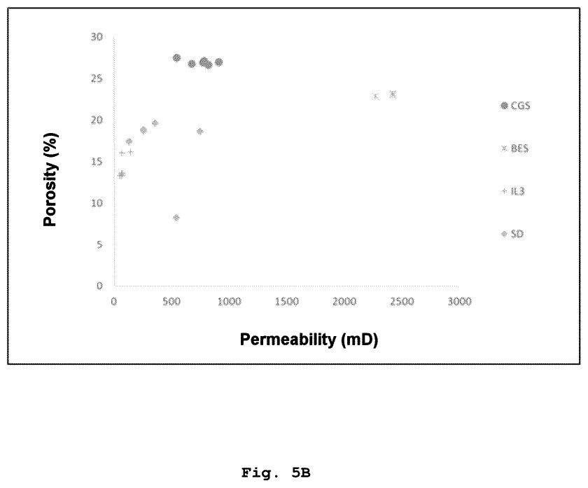

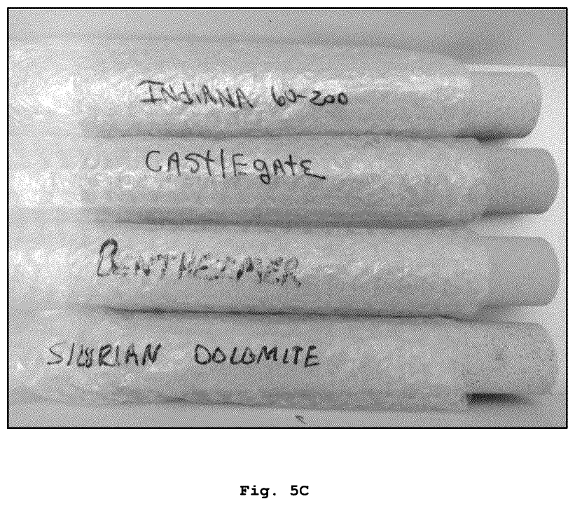

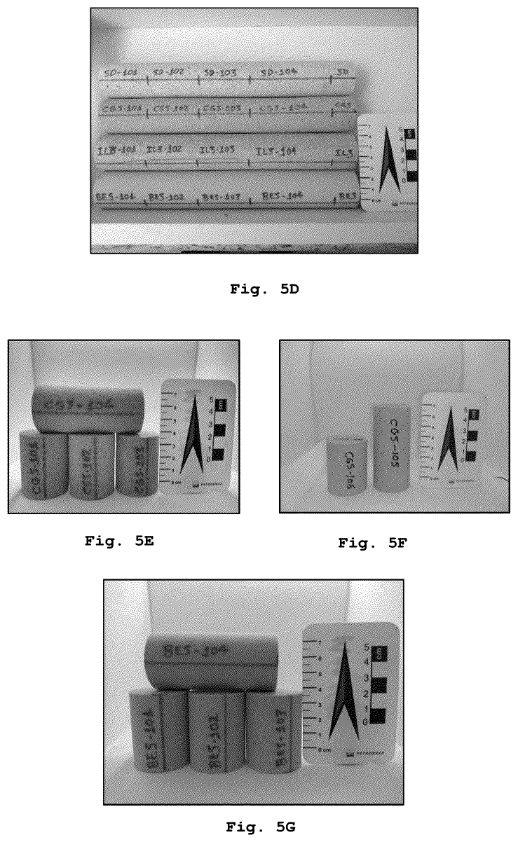

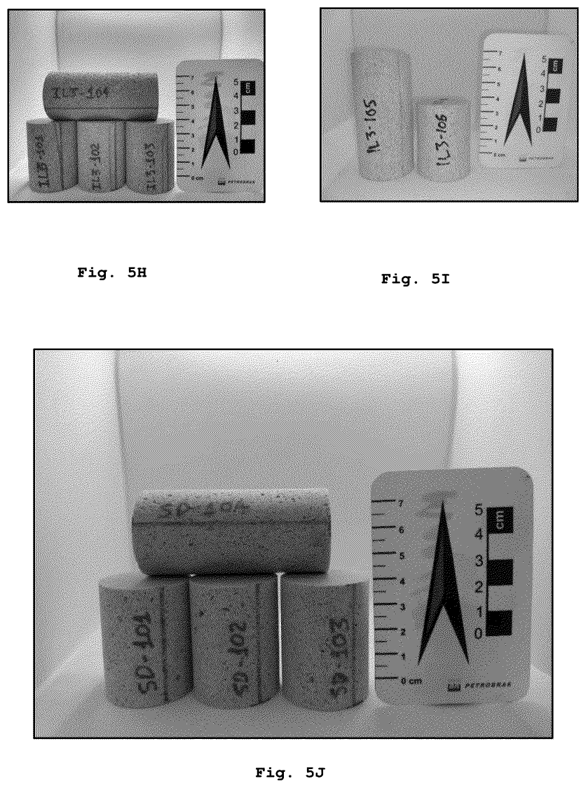

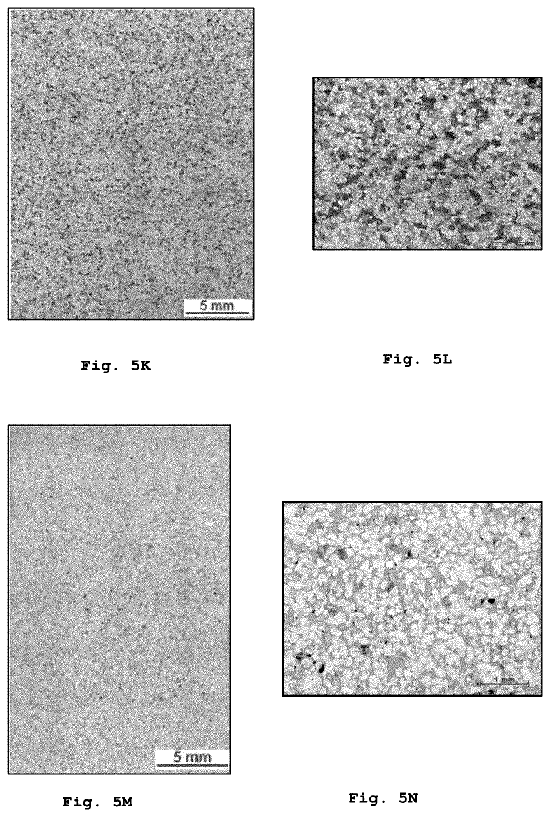

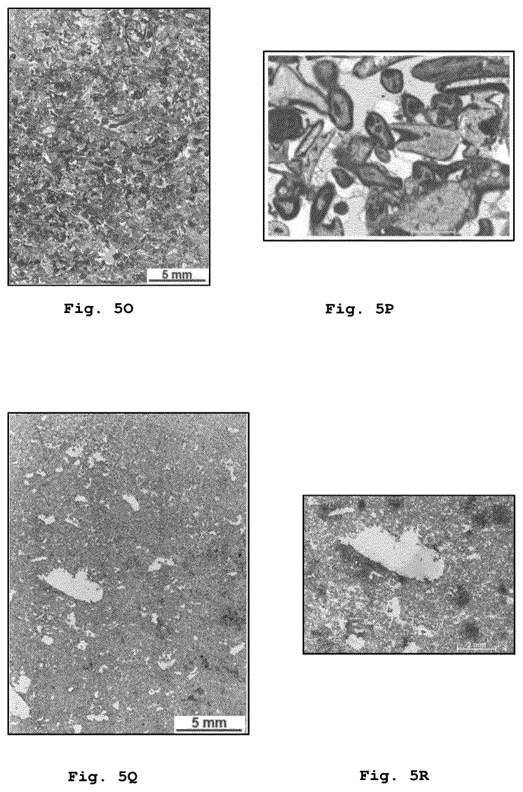

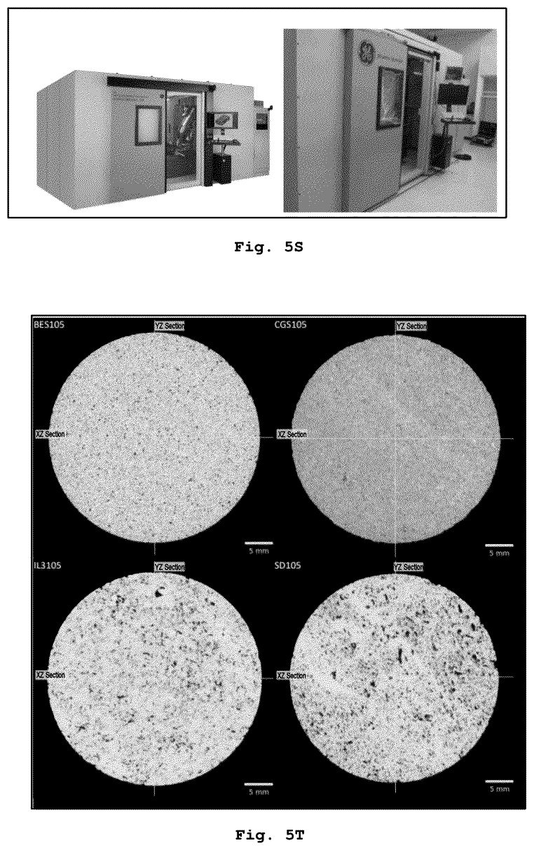

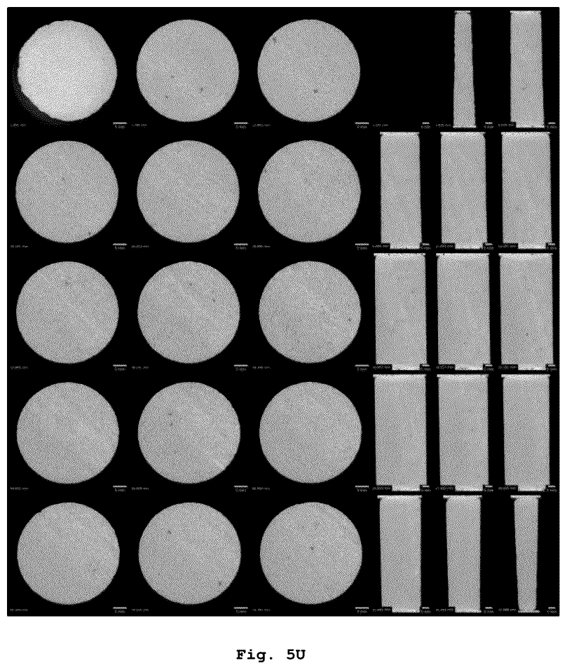

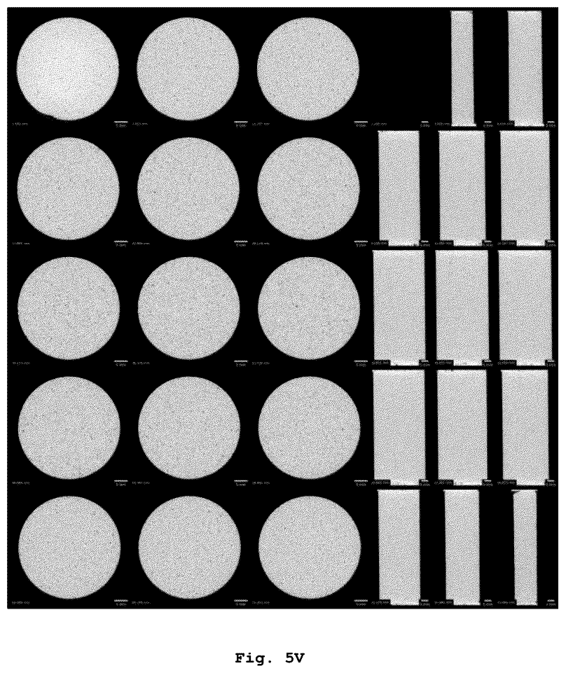

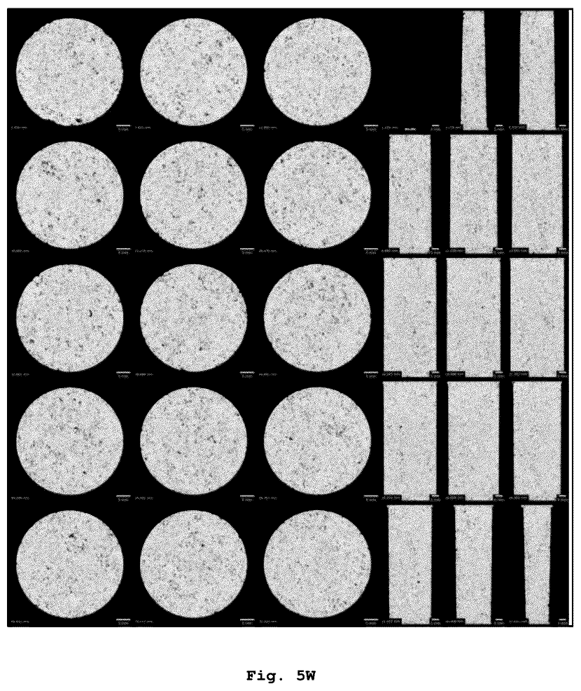

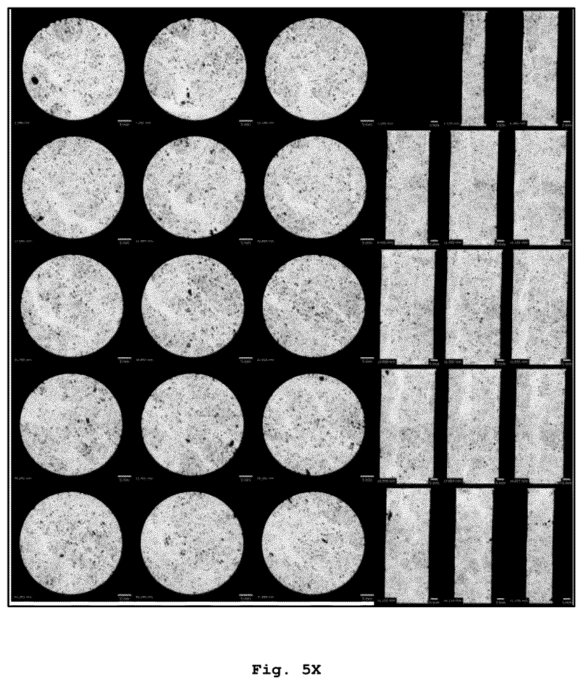

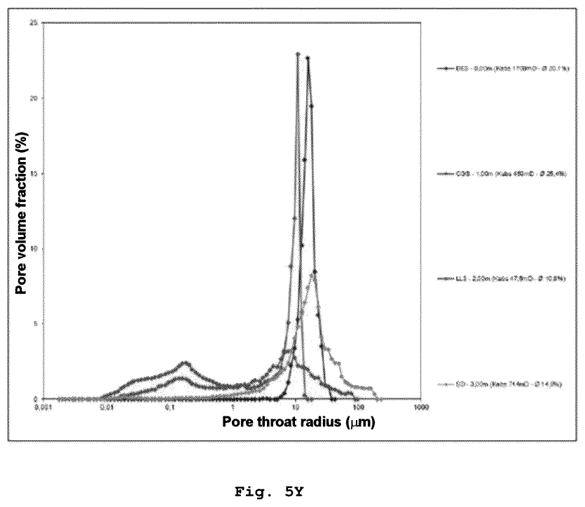

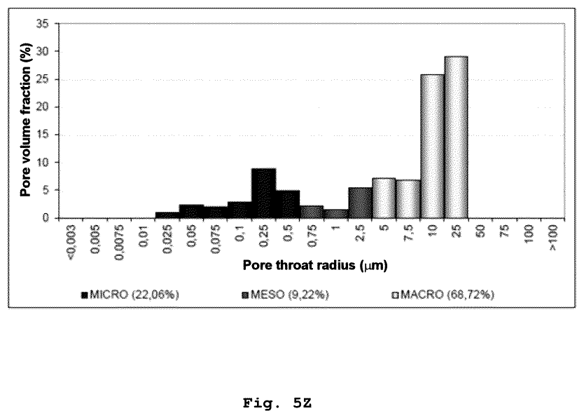

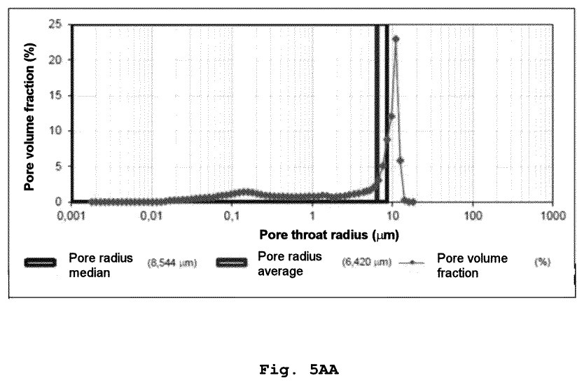

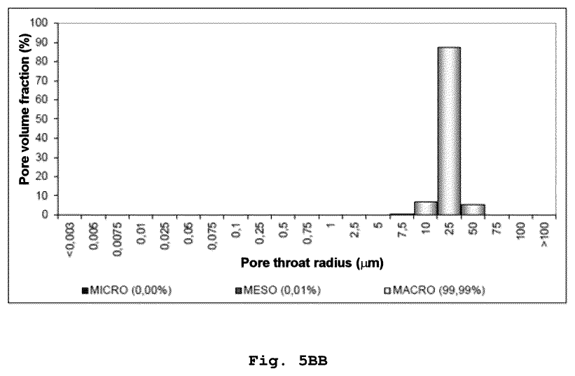

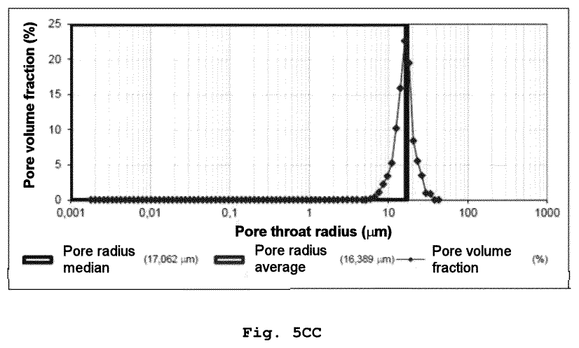

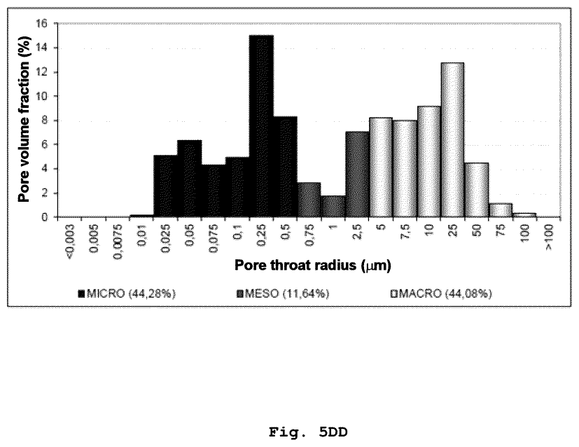

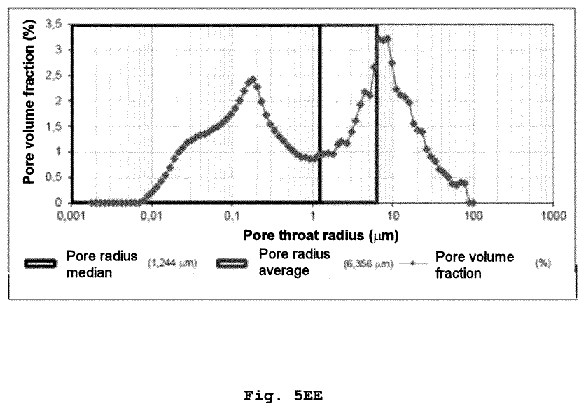

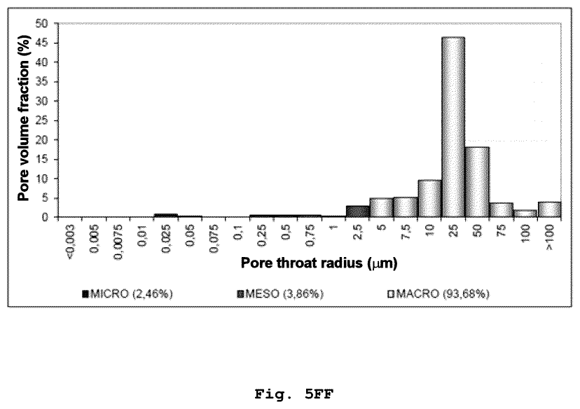

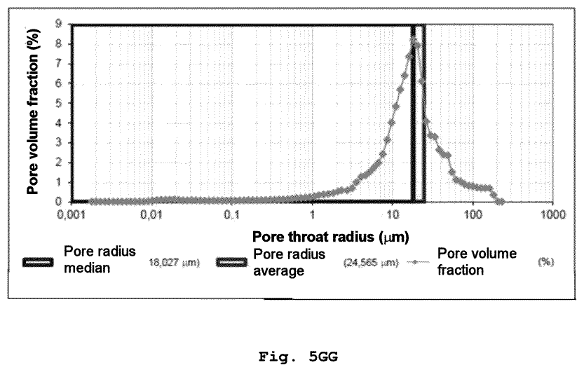

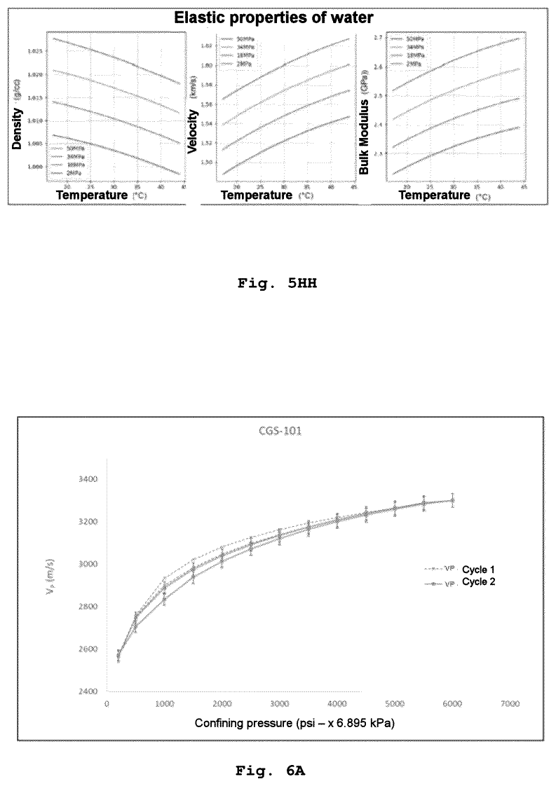

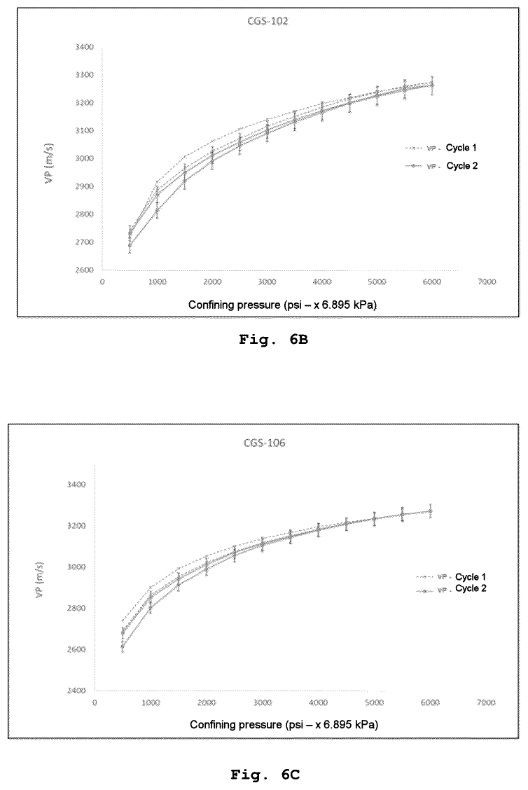

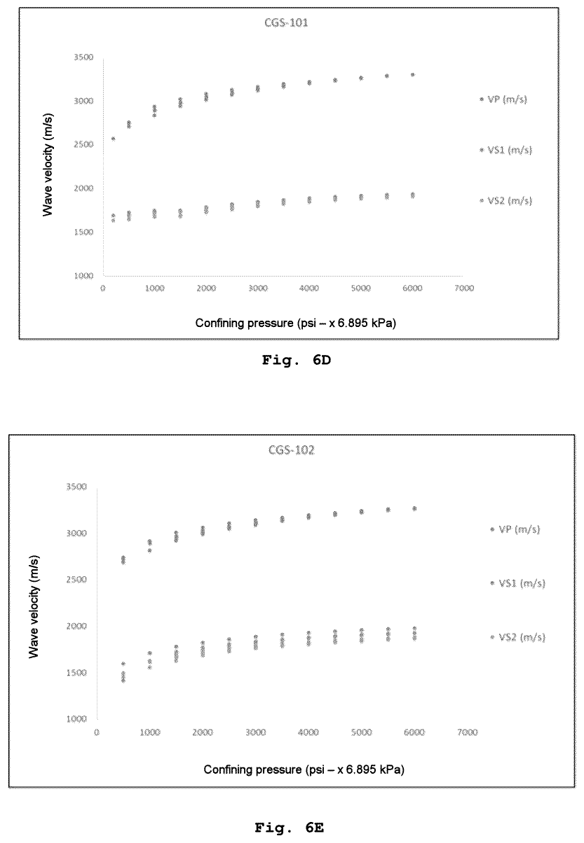

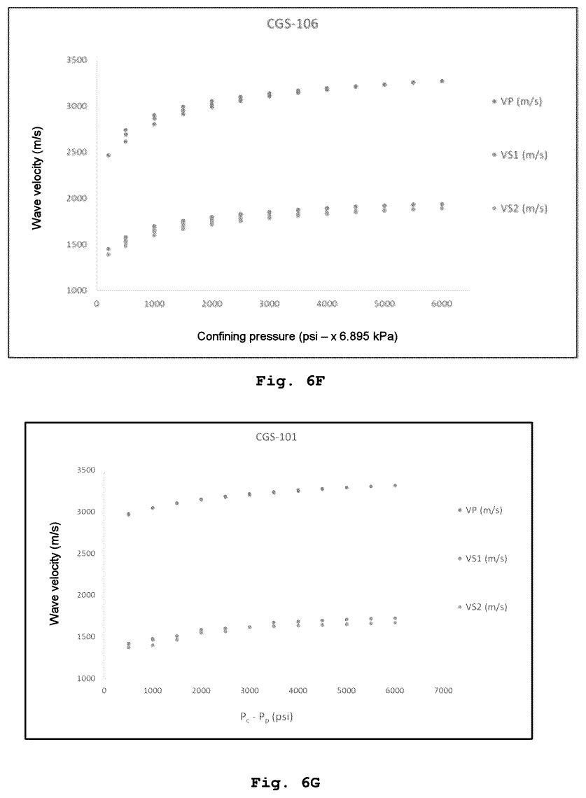

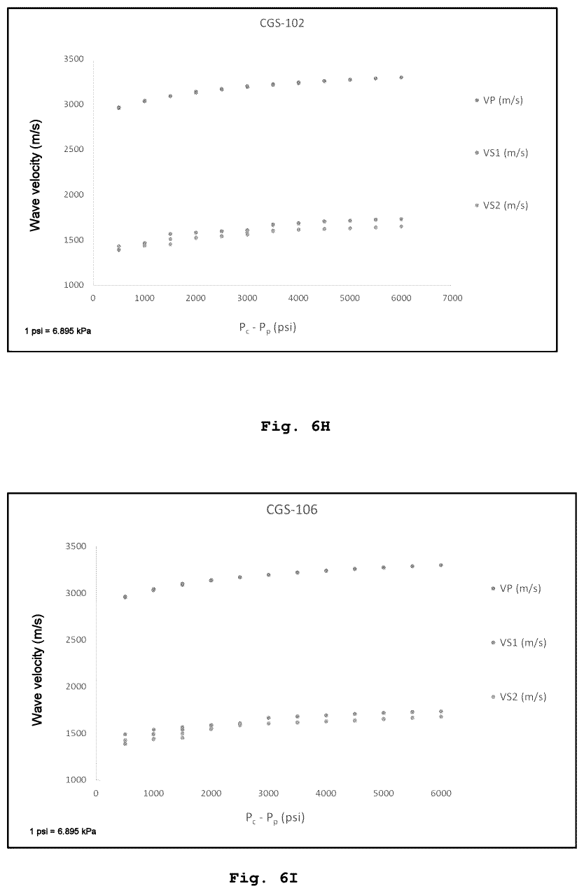

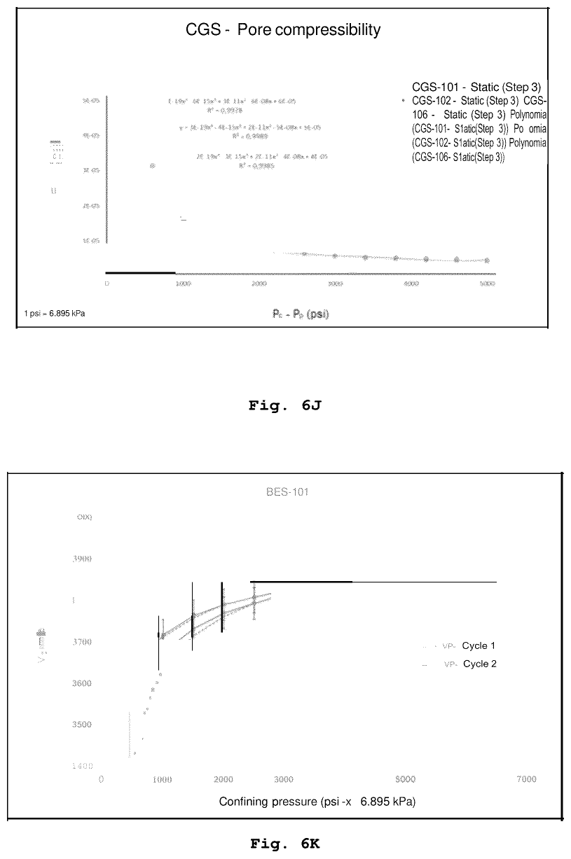

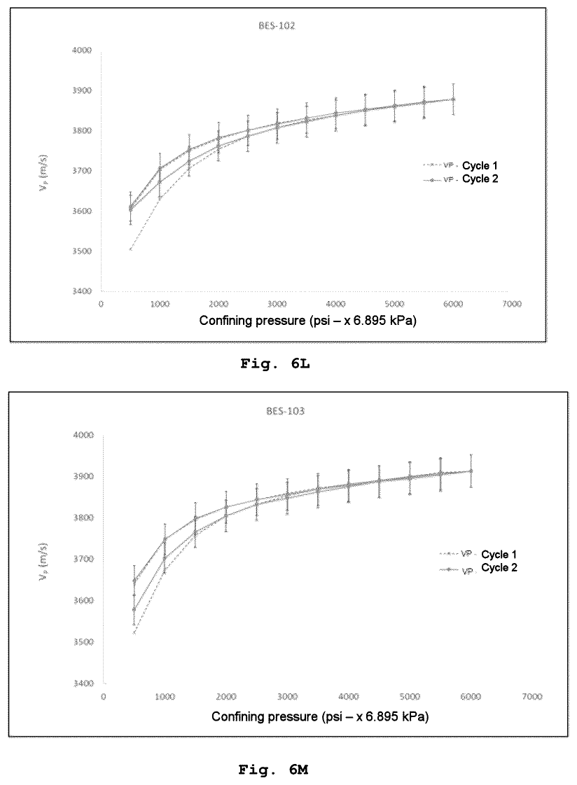

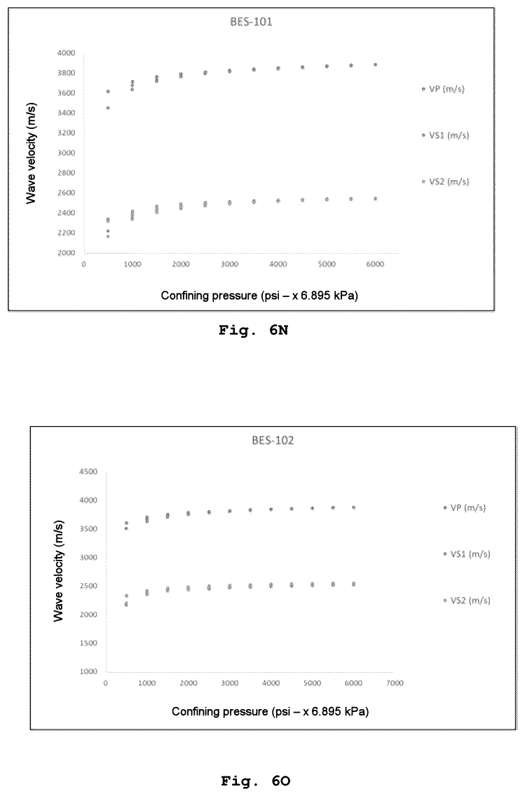

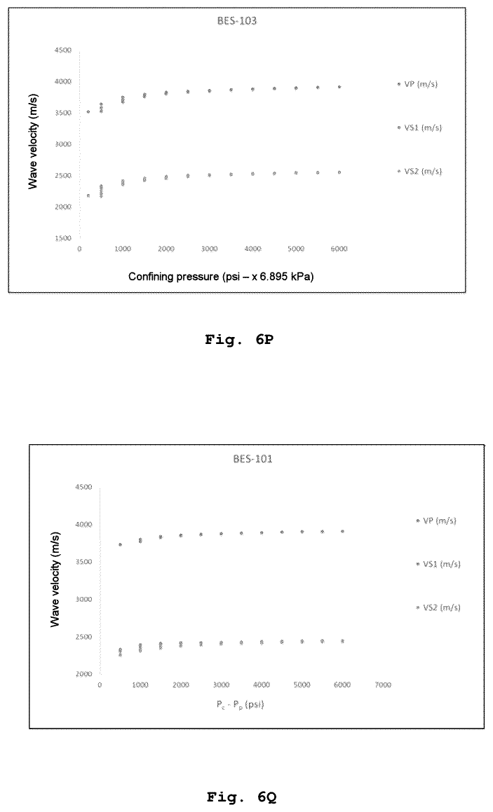

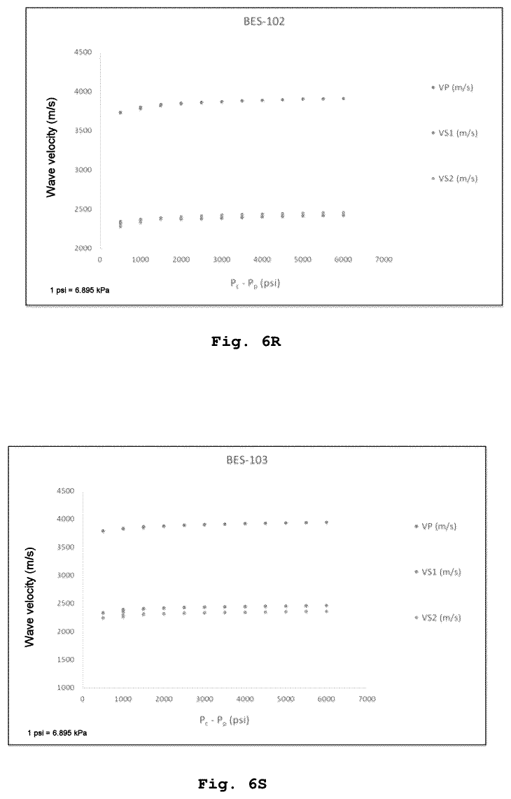

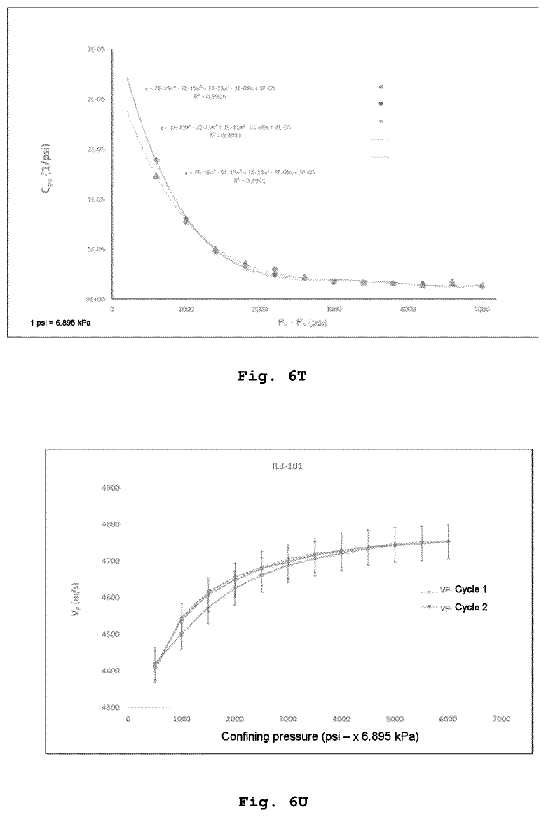

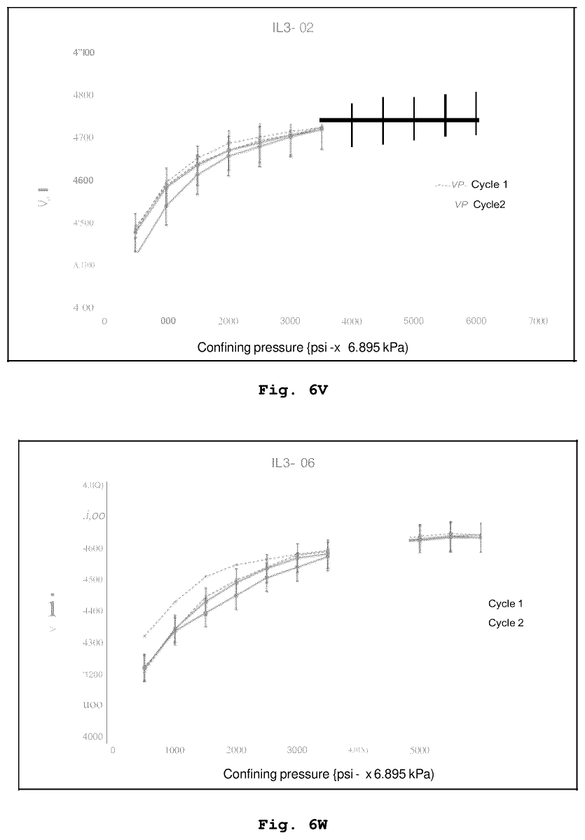

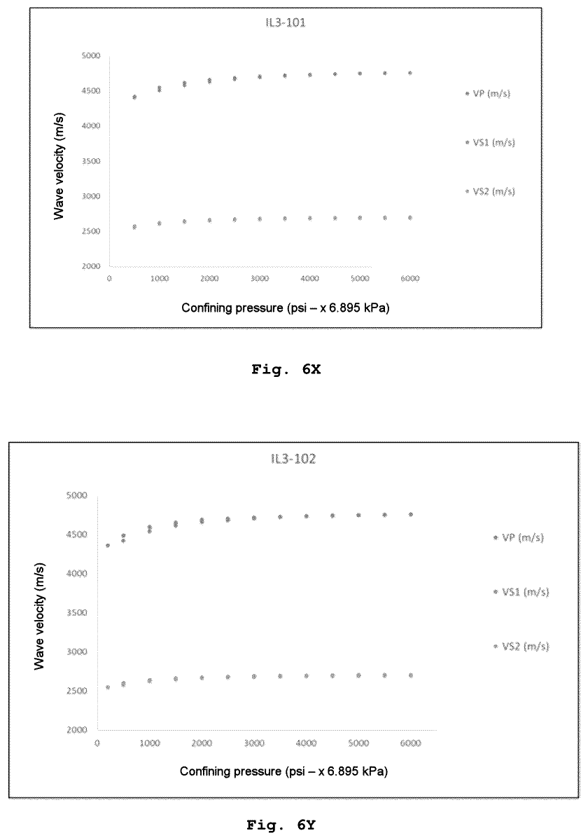

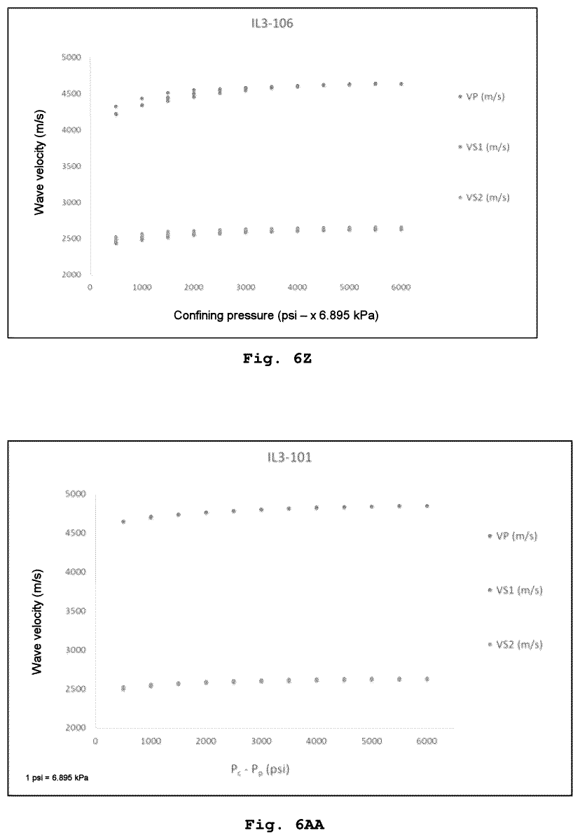

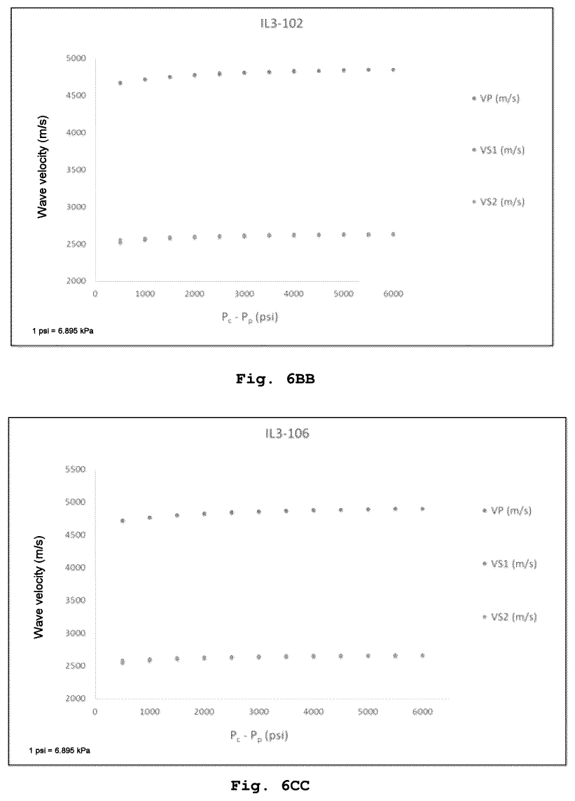

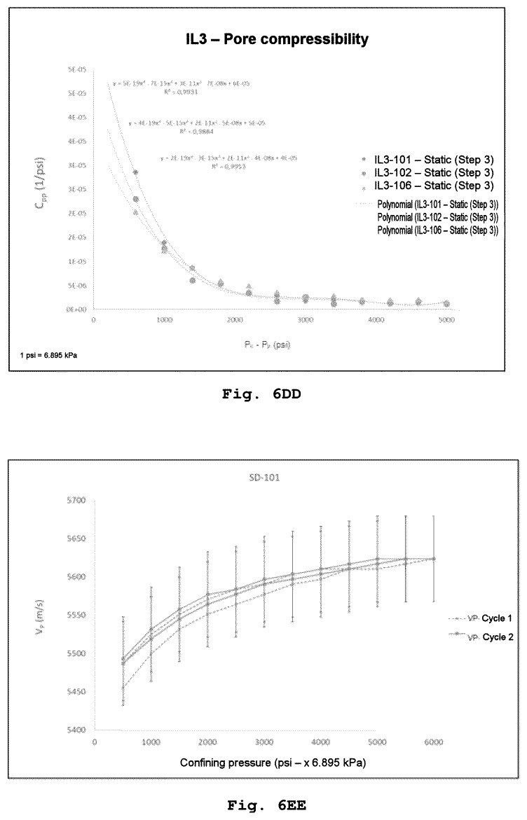

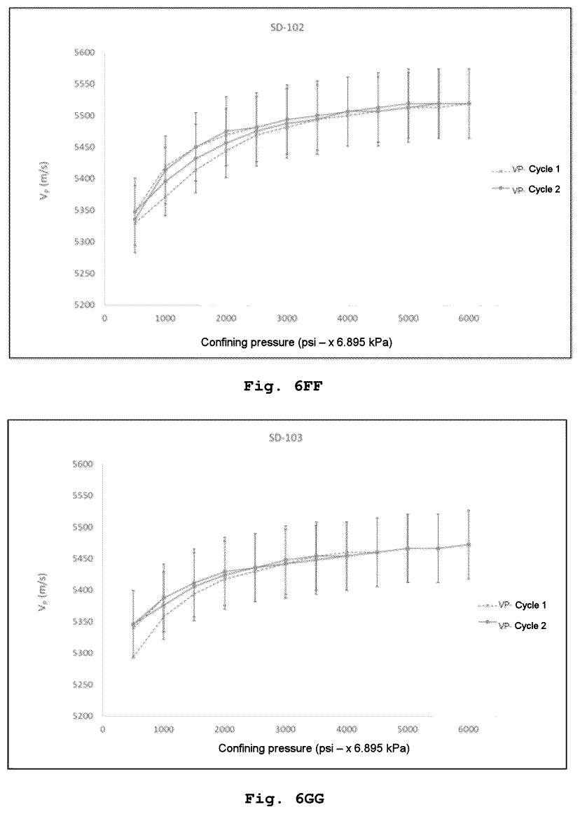

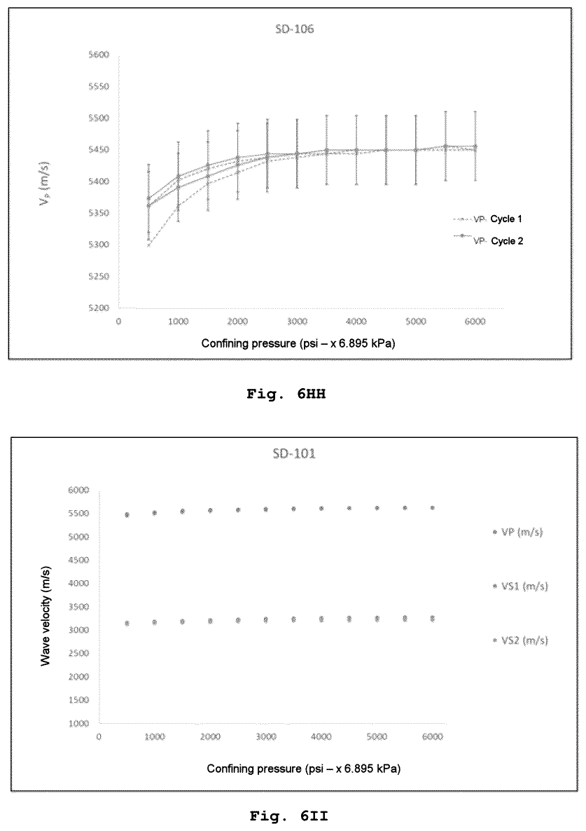

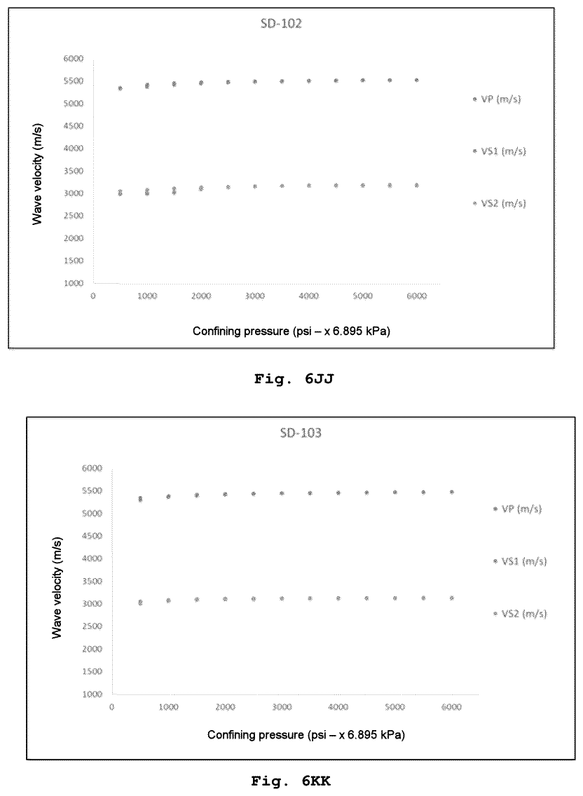

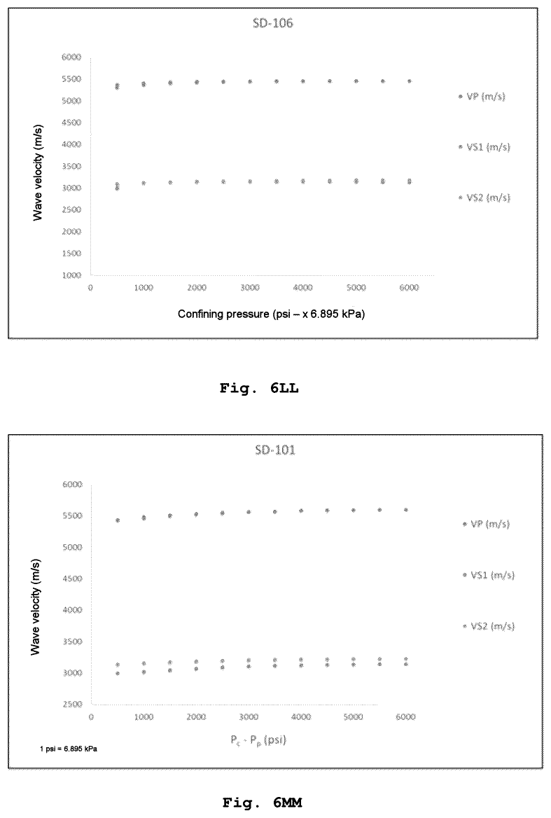

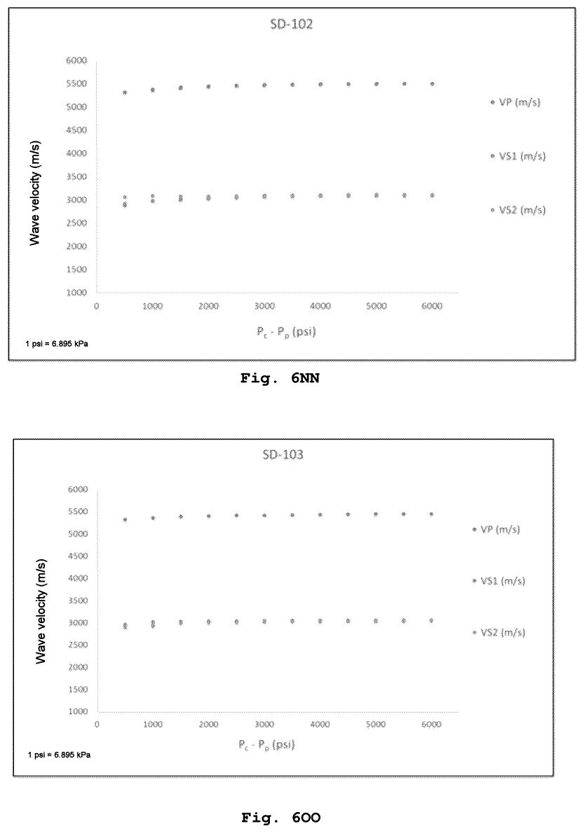

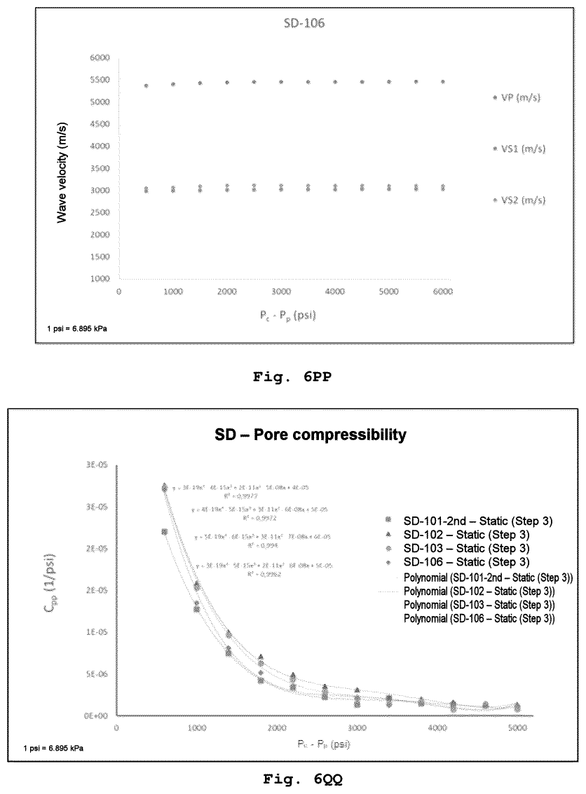

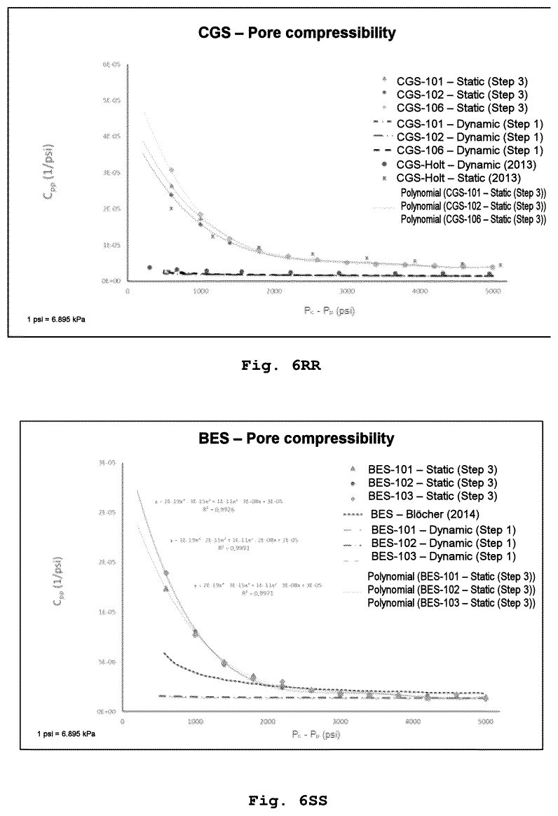

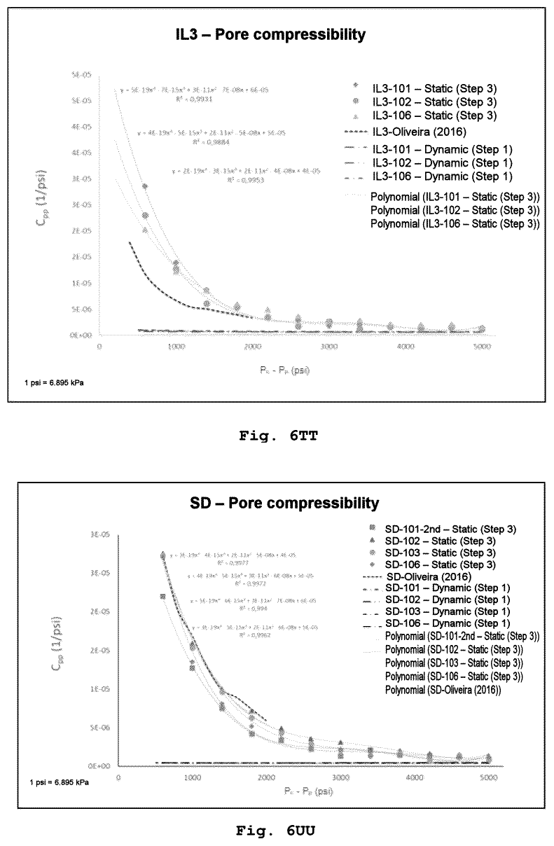

FIG. 3P—Applied confining pressure variation cycles and resulting pore pressure variation values for calculating the Skempton coefficient after the second flow of five pore volumes in the sample CGS-103.

FIG. 3Q—Applied confining pressure variation cycles and resulting pore pressure variation values for calculating the Skempton coefficient after the first flow of five pore volumes in the sample IL3-103.

FIG. 3R—Applied confining pressure variation cycles and resulting pore pressure variation values for calculating the Skempton coefficient after the second flow of five pore volumes in the sample IL3-103.

FIG. 3S—Sample CGS-103—Cₚₚ values (without volume correction) calculated for two depletion rates considered most relevant for evaluation with pressure difference limited to 5800 psi (39.99 MPa).

FIG. 3T—Sample CGS-103—Cₚₚ values (without volume correction) calculated for two depletion rates considered most relevant for evaluation with a pressure difference limited to 5800 psi (39.99 MPa).

FIG. 3U—Sample IL3-103—Cₚₚ values (without volume correction) calculated for four depletion rates considered most relevant for evaluation with pressure difference limited to 5800 psi (39.99 MPa).

FIG. 3V—Sample IL3-103—Cₚₚ values (without volume correction) calculated for four depletion rates considered most relevant for evaluation with pressure difference limited to 5000 psi (34.474 MPa).

FIG. 3W—Sample SD-101—Cₚₚ values calculated for two depletion rates considered most relevant for evaluation with pressure difference limited to 5000 psi (34.474 MPa).

FIG. 4A—Overview of the equipment used in the experimental methodology.

FIG. 4B—Schematic drawing of the equipment used in the tests.

FIG. 4C—TEMCO pressure vessel with acoustic velocity transducers.

FIG. 4D and FIG. 4E—Sample inside the vessel after the test (on the left—FIG. 4D) and the lining material where the sample is inserted to isolate the same from the confining fluid (on the right—FIG. 4E).

FIG. 4F and FIG. 4G—Inside the vessel, there are two acoustic AVT velocity transducers.

FIG. 4H—Schematic drawing of the experimental configuration: vessel, AVTs, pumps, lines and valves (adapted from Vasquez et al. [53]).

FIG. 4I and FIG. 4J—Detail of the AVTs (on the left—FIG. 4I) and internal part of an AVT (on the right—FIG. 4J).

FIG. 4K—oscilloscope (Agile Technologies) indicating the “P” waveforms in dry (yellow) and saturated (gray) samples.

FIG. 4L—pulse generator (Olympus).

FIG. 4M and FIG. 4N—Syringe pump (on the left—FIG. 4M) and manual control panel (on the right —FIG. 4N) (Teledyne ISCO—Manufacturer's manual).

FIG. 4O and FIG. 4P—Confining pressure pump Teledyne ISCO 100DX (on the left—FIG. 4O)/Pore pressure pump—Teledyne ISCO 260D (on the right—FIG. 4P).

FIG. 4Q—Screens linked to the automated system computer (example).

FIG. 4R—Main equipment involved in the calibration process.

FIG. 4S—Calibration function of values measured in the pump (AVT-AVT).

FIG. 4T—Volume variation due to pore pressure variation.

FIG. 4U and FIG. 4V—Aluminum sample used to calibrate the system.

FIG. 4W—Cₚₚ of aluminum measured and adjusted (after calibration correction) for two different volumes in the system (2 ml and 10 ml).

FIG. 5A—Graph relating porosity and Young's modulus of outcrop samples available for this study. Each point represents a different lithotype (sandstones and carbonates).

FIG. 5B—Porosity and permeability data for each selected outcrop sample.

FIG. 5C—Samples before cutting.

FIG. 5D—Samples marked and named before cutting.

FIG. 5E and FIG. 5F—Castlegate Sandstone (CGS) samples after cutting.

FIG. 5G—Bentheimer Sandstone (BES) samples after cutting.

FIG. 5H and FIG. 5I—Indiana limestone samples (IL3) after cutting.

FIG. 5J—Silurian Dolomite (SD) samples after cutting.

FIG. 5K and FIG. 5L—General view of the thin section on the left and closer view on the right indicating intergranular porosity—Castlegate Sandstone.

FIG. 5M and FIG. 5N—General view of the thin section on the left and a closer view on the right indicating intergranular porosity—Bentheimer Sandstone.

FIG. 5O and FIG. 5P—General view of the thin section on the left and closer view on the right indicating intragranular porosity—Indiana Limestone.

FIG. 5Q and FIG. 5R—General view of the thin section on the left (FIG. 5Q) and a closer view on the right (FIG. 5R) indicating vugular porosity—Silurian Dolomite.

FIG. 5S—Photos of the used microtomography [49].

FIG. 5T—Microtomography—Cross sections of the samples used (BES, CGS, IL3 and SD).

FIG. 5U—Microtomography—Cross and longitudinal sections of Castlegate Sandstone (CGS) samples with 51 μm resolution.

FIG. 5V—Microtomography—Cross and longitudinal sections of the Indiana limestone sample (IL3) with 51 μm resolution.

FIG. 5W—Microtomography—Cross and longitudinal sections of the Indiana limestone sample (IL3) with 51 μm resolution.

FIG. 5X—Microtomography—Cross and longitudinal sections of the Silurian Dolomite (SD) sample with 51 μm resolution.

FIG. 5Y—Distribution of pore throat radii values per pore volume fraction for each of the used lithologies.

FIG. 5Z—Histogram indicating the distribution of the pore throat radius values of the Castlegate Sandstone sample.

FIG. 5AA—Distribution of pore throat radius values per pore volume fraction of the Castlegate Sandstone sample.

FIG. 5BB—Histogram indicating the distribution of the pore throat radius values of the Bentheimer Sandstone sample.

FIG. 5CC—Distribution of pore throat radius values per pore volume fraction of the Bentheimer Sandstone sample.

FIG. 5DD—Histogram indicating the distribution of the pore throat radius values of the Indiana limestone sample.

FIG. 5EE—Distribution of pore throat radius values per pore volume fraction of the Indiana limestone sample.

FIG. 5FF—Histogram indicating the distribution of the pore throat radius values of the Silurian Dolomite sample.

FIG. 5GG—Distribution of pore throat radius values per pore volume fraction of the Silurian Dolomite sample.

FIG. 5HH—Properties of the water used in the tests.

FIG. 6A—CGS-101—Dry test—Compressive wave velocities (Vₚ) in loading-unloading cycles (the vertical bars indicate the expected error of ±1%).

FIG. 6B—CGS-102—Dry test—Compressive wave velocities (Vₚ) in loading-unloading cycles (the vertical bars indicate expected error of ±1%).

FIG. 6C—CGS-106—Dry test—Compressive wave velocities (Vₚ) in loading-unloading cycles (the vertical bars indicate expected error of ±1%).

FIG. 6D—CGS-101—Dry test—Compressive wave velocities (Vₚ) and shear velocities (VS1 and VS2) in both loading and unloading cycles.

FIG. 6E—CGS-102—Dry test—Compressive wave velocities (Vₚ) and shear wave velocities (VS1 and VS2) in both loading and unloading cycles.

FIG. 6F—CGS-106—Dry Test—Compressive wave velocities (Vₚ) and shear wave velocities (VS1 and VS2) in both loading and unloading cycles.

FIG. 6G—CGS-101—Saturated test—Compressive wave velocities (Vₚ) and shear wave velocities (VS1 and VS2) in both loading and unloading cycles.

FIG. 6H—CGS-102—Saturated test—Compressive wave velocities (Vₚ) and shear wave velocities (VS1 and VS2) in both loading and unloading cycles.

FIG. 6I—CGS-106—Saturated test—Compressive wave velocities (Vₚ) and shear wave velocities (VS1 and VS2) in both loading and unloading cycles.

FIG. 6J—CGS—Pore compressibility values (Cpp) obtained in step 3 of the tests and curves obtained by polynomial regression for each of the three samples.

FIG. 6K—BES-101—Dry test—Compressive wave velocities (Vₚ) in both loading and unloading cycles (the vertical bars indicate the expected error of ±1%).

FIG. 6L—BES-102—Dry test—Compressive wave velocities (Vₚ) in both loading and unloading cycles (the vertical bars indicate the expected error of ±1%).

FIG. 6M—BES-103—Dry test—Compressive wave velocities (Vₚ) in both loading and unloading cycles (the vertical bars indicate the expected error of ±1%).

FIG. 6N—BES-101—Dry test—Compressive wave velocities (Vₚ) and shear wave velocities (VS1 and VS2) in both loading and unloading cycles.

FIG. 6O—BES-102—Dry test—Compressive wave velocities (Vₚ) and shear wave velocities (VS1 and VS2) in both loading and unloading cycles.

FIG. 6P—BES-103—Dry test—Compressive wave velocities (Vₚ) and shear wave velocities (VS1 and VS2) in both loading and unloading cycles.

FIG. 6Q—BES-101—Saturated Test —Compressive wave velocities (Vₚ) and shear wave velocities (VS1 and VS2) in both loading and unloading cycles.

FIG. 6R—BES-102—Saturated test—Compressive wave velocities (Vₚ) and shear wave velocities (VS1 and VS2) in both loading and unloading cycles.

FIG. 6S—BES-103—Saturated test—Compressive wave velocities (Vₚ) and shear wave velocities (VS1 and VS2) in both loading and unloading cycles.

FIG. 6T—BES—Pore compressibility values (Cpp) obtained in step 3 of the tests and curves obtained by polynomial regression for each of the three samples.

FIG. 6U—IL3-101—Dry test—Compressive wave velocities (Vₚ) in both loading and unloading cycles (the vertical bars indicate the expected error of ±1%).

FIG. 6V—IL3-102—Dry test—Compressive wave velocities (Vₚ) in both loading and unloading cycles (the vertical bars indicate the expected error of ±1%).

FIG. 6W—IL3-106—Dry test—Compressive wave velocities (Vₚ) in both loading and unloading cycles (the vertical bars indicate the expected error of ±1%).

FIG. 6X—IL3-101—Dry test—Compressive wave velocities (Vₚ) and shear wave velocities (VS1 and VS2) in both loading and unloading cycles.

FIG. 6Y—IL3-102—Compressive wave velocities (Vₚ) and shear wave velocities (VS1 and VS2) in both loading and unloading cycles.

FIG. 6Z—IL3-106—Dry test—Compressive wave velocities (Vₚ) and shear wave velocities (VS1 and VS2) in both loading and unloading cycles.

FIG. 6AA—IL3-101—Saturated test—Compressive wave velocities (Vₚ) and shear wave velocities (VS1 and VS2) in both loading and unloading cycles.

FIG. 6BB—IL3-102—Saturated test—Compressive wave velocities (Vₚ) and shear wave velocities (VS1 and VS2) in both loading and unloading cycles.

FIG. 6CC—IL3-106—Saturated test—Compressive wave velocities (Vₚ) and shear wave velocities (VS1 and VS2) in both loading and unloading cycles.

FIG. 6DD—IL3—Pore compressibility values (Cpp) obtained in step 3 of the tests and curves obtained by polynomial regression for each of the three samples.

FIG. 6EE—SD-101-2nd—Dry Test—Compressive wave velocity (Vₚ) in the two loading and unloading cycles (the vertical bars indicate the expected error of ±1%).

FIG. 6FF—SD-102—Dry Test—Compressive wave velocity (Vₚ) in the two loading and unloading cycles (the vertical bars indicate the expected error of ±1%).

FIG. 6GG—SD-103—Dry Test—Compressive wave velocity (Vₚ) in the two loading and unloading cycles (the vertical bars indicate the expected error of ±1%).

FIG. 6HH—SD-106—Dry Test—Compressive wave velocity (Vₚ) in the two loading and unloading cycles (The vertical bars indicate the expected error of ±1%).

FIG. 611—SD-101-2nd—Dry Test—Compressive wave velocities (Vₚ) and shear wave velocities (VS1 and VS2) in both loading and unloading cycles.

FIG. 6JJ—SD-102—Dry Test—Compressive wave velocities (Vₚ) and shear wave velocities (VS1 and VS2) in the two loading and unloading cycles.

FIG. 6KK—SD-103—Dry Test—Compressive wave velocities (Vₚ) and shear wave velocities (VS1 and VS2) in the two loading and unloading cycles.

FIG. 6LL—SD-106—Dry Test—Compressive wave velocities (Vₚ) and shear wave velocities (VS1 and VS2) in the two loading and unloading cycles.

FIG. 6MM—SD-101-2nd—Saturated Test —Compressive wave velocities (Vₚ) and shear wave velocities (VS1 and VS2) in both loading and unloading cycles.

FIG. 6NN—SD-102—Saturated Test —Compressive wave velocities (Vₚ) and shear wave velocities (VS1 and VS2) in the two loading and unloading cycles.

FIG. 600—SD-103—Saturated Test —Compressive wave velocities (Vₚ) and shear wave velocities (VS1 and VS2) in the two loading and unloading cycles.

FIG. 6Pp—SD-106—Saturated Test —Compressive wave velocities (Vₚ) and shear wave velocities (VS1 and VS2) in the two loading and unloading cycles.

FIG. 6QQ—SD—Pore compressibility values (Cₚₚ) obtained in Step 3 of the tests, and curves obtained by polynomial regression for each of the four samples.

FIG. 6RR—CGS—Cₚₚ results with static and dynamic tests. Comparison of results with those obtained in the literature.

FIG. 6SS—BES—Cₚₚ results of static and dynamic tests. Comparison with results obtained in the literature.

FIG. 6TT—IL3—Cₚₚ static and dynamic tests. Comparison of results with those obtained in the literature.

FIG. 6UU—SD—Cₚₚ results of dynamic and static tests. Comparison with those obtained in the literature.

### DETAILED DESCRIPTION

The present invention relates to a method and system for determining static and dynamic elastic and poroelastic parameters of rocks under hydrostatic confining pressure conditions.

The method aims at obtaining correlations between static and dynamic values of parameters related to the poroelastic characterization of oil reservoir rocks, using both dynamic tests—through the propagation of the compression wave (P) and shear wave (S)—and static tests. Furthermore, dynamic elastic parameters such as E, n and K are obtained for dry and saturated samples and Gassmann relations can be verified.

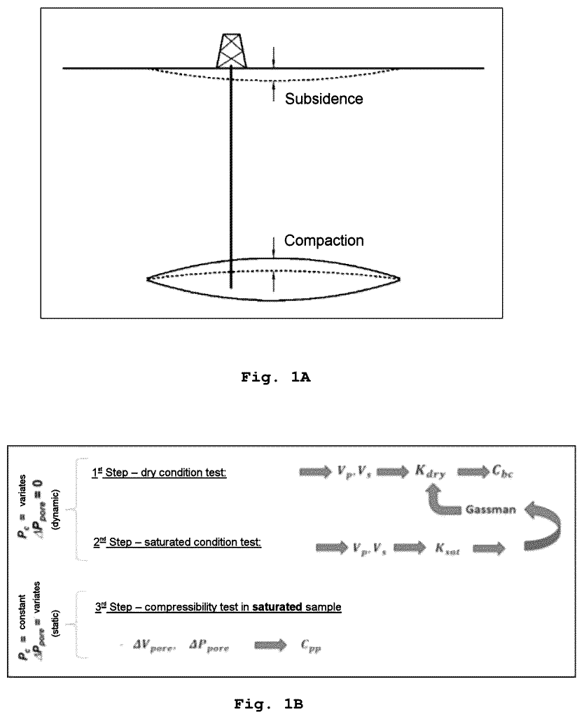

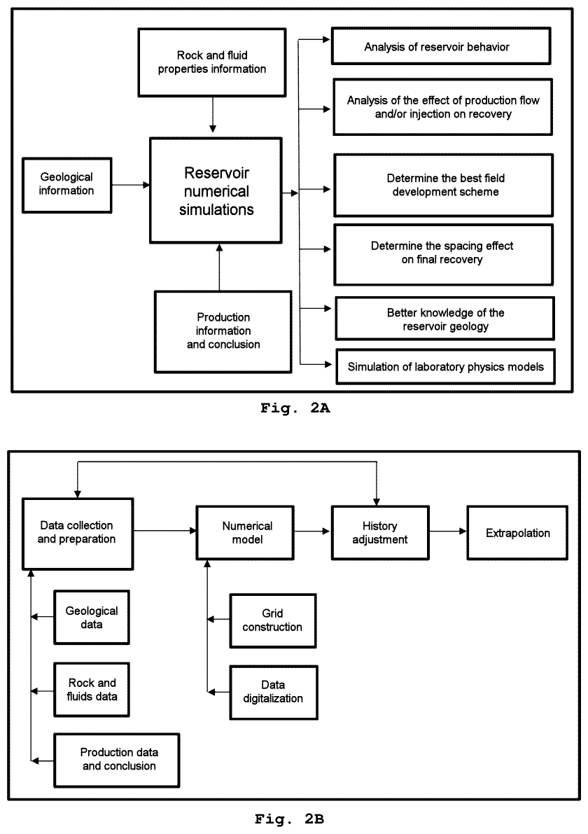

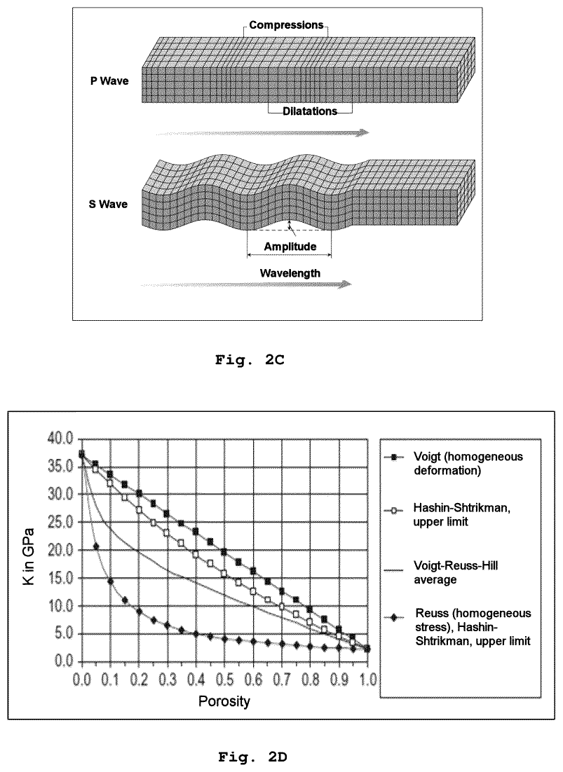

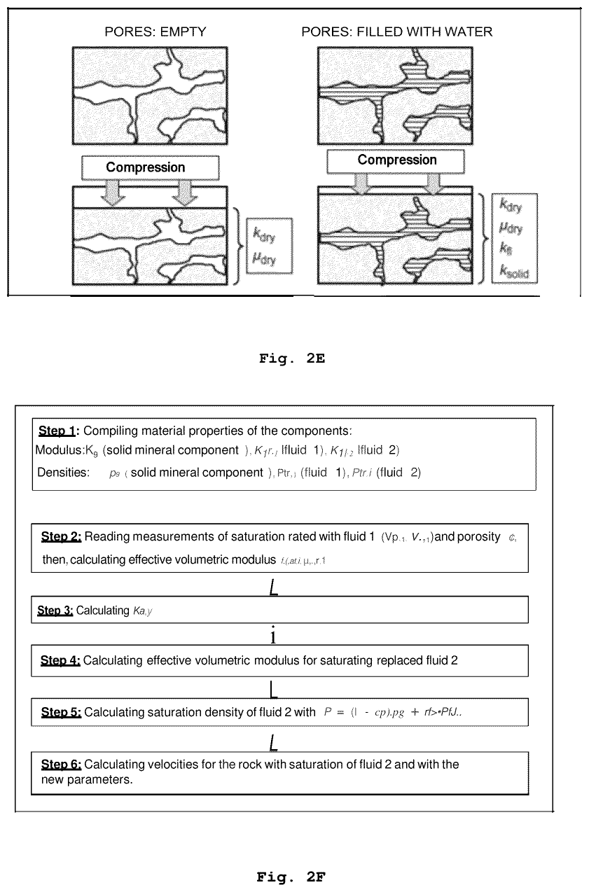

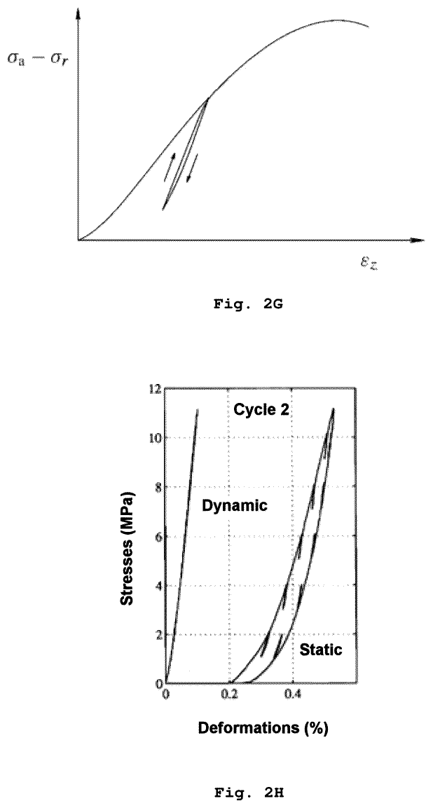

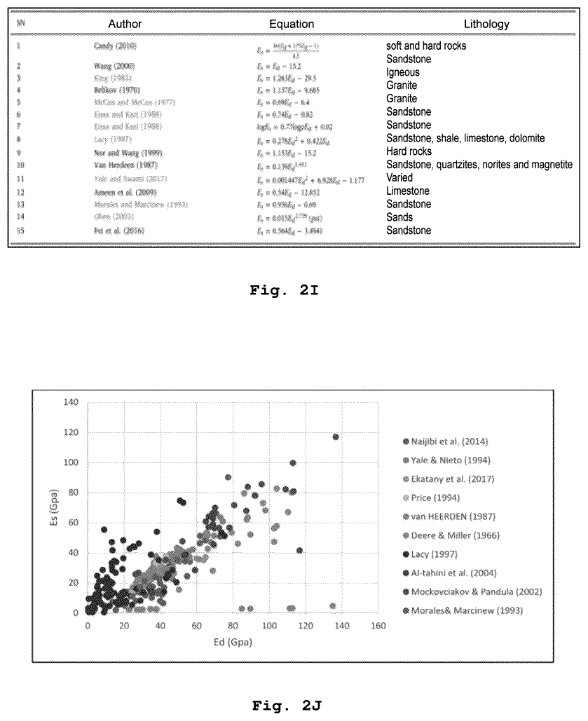

The methodology consists of testing the same sample in dry and saturated conditions using an experimental configuration applying three test steps, as illustrated in FIG. 1.2. This process leads to results of dynamic pore compressibility values using the Vₚ and V_{S} results (1^{st} Step) and static pore compressibility values (3^{rd} Step).

In addition, other poroelastic parameters (the Biot and Skempton coefficients) are acquired using this methodology. Dynamic elastic parameters such as Young's modulus, Poisson's ratio and volumetric compressibility are outputs of the same process in both dry (1^{st} Step) and saturated (2^{nd} Step) conditions, and the comparison of these results can be used to evaluate the theoretical replacement of the fluid using the Gassmann relations.

It is noteworthy that, although correlations have been obtained for several elastic parameters, there is still a lack of tests that can correlate poroelastic parameters such as pore compressibility (Cₚₚ) acquired in dynamic tests with static tests.

Therefore, the present invention mainly contributes to: improving the determination of poroelastic parameters for use in flow simulators and evaluation of 4D seismic results, using dynamic and static tests to provide estimates of geomechanical properties of rocks.

Furthermore, the present invention presents at least the following advantages and differences in relation to existing methods:
Acquisition of consistent data on the propagation velocity of elastic waves in the same rock sample in both dry and saturated conditions;
In just one test, pore compressibility results are obtained by dynamic and static methods, thus providing information for correlations of this parameter for each lithology intended to be tested;
Obtaining elastic and poroelastic parameters in the same test;
Evaluation of results of elastic parameters in saturated samples and comparison of the same with those predicted in theory;
Testing time of up to 12 hours in total (which can be divided into distinct steps) with several automated steps.

The features and advantages of the present invention will clearly emerge from the following detailed description and with reference to the accompanying drawings, these being provided only as preferred and non-limiting illustrations.

In a preferred embodiment of the present invention, a method for determining static and dynamic elastic and poroelastic parameters of rocks is disclosed.

In schematic form, FIG. 1.2 presents a summary of each step of the method, with the main obtained data and the calculated values.

The first step directly measures Vₚ and Vₛ to obtain the dynamic parameters K_{dry} and dynamic Cₚₚ in a dry sample, varying the confining pressure and maintaining the pore pressure equal to the atmospheric pressure. After the first step of testing, a saturation process was carried out and the Skempton coefficients B were obtained.

The second step is performed with the saturated sample and directly measures the V_{P} and V_{S} to obtain the volumetric deformation modulus of the saturated rock Kₛₐₜ (and other elastic parameters) in a saturated sample, varying the confining pressure and maintaining the pore pressure equal to the atmospheric pressure. With the results of Steps 1 and 2, K can be calculated with the fluid replacement proposed by Gassmann and the results compared.

For the third step, the confining pressure is maintained at a certain level and the pore pressure is varied at a constant reduction rate, obtaining the value of the static parameter Cₚₚ.

The above methodology was developed and tested consistently for different types of samples, presenting good results, both in terms of quality and execution time.

In general, the results of the empirical methods to obtain the pore compressibility showed significant differences of 230 to 436%, when compared with the static measurements of the third step of the proposed methodology, confirming that they should be avoided for use in reservoir simulations. This empirical data should only be used when there is no better information and can be corrected as quickly as possible with data generated from laboratory tests.

The results of static pore compressibilities (Cₚₚ) obtained with the proposed methodology, when compared to those in the literature, are similar and prove that the methodology can be used to achieve reliable results.

Correlations were also obtained between pore compressibility data through static and dynamic tests for the lithologies of interest. Dynamic tests have the advantage of being non-destructive and represent many tests already carried out, whether on samples or sonic profiles in wells.

Thus, the results support that the present invention provides an improvement in the determination of poroelastic parameters that can be used, at least, in flow simulators and in evaluations of 4D seismic results.

Next, the steps of the methodology proposed by the present invention will be discussed in detail.

The method for determining static and dynamic elastic and poroelastic parameters of rocks under hydrostatic confining pressure conditions is characterized in that it comprises, in a first step, carrying out a first dynamic test with a dry sample inside a pressure vessel; in a second step, said dry sample is saturated with deaerated water and a second dynamic test is carried out with the saturated sample and constant pore pressure; and in a third step, carrying out a third static compressibility test on said saturated sample.

### First Step—Dynamic Test in Dry Sample Condition

The first step preferably comprises the steps of:
a) Applying a loading and unloading cycle, at predefined pressure increments, over a confining pressure range P_{c} with loading and unloading paths, wherein the initial Pₚ value is equal to the atmospheric pressure;
b) Obtaining values of Vₚ and Vₛ for each of the predefined pressure increments of the confining pressure P_{c} in the cycle;
c) Calculating the elastic parameters E_{dry} (Equation 31), v_{dry} (Equation 32) and K_{dry} (Equation 33) and the poroelastic parameter Cₚₚ (Equation 16) of the cycle;
d) Repeating steps a) to c) to obtain the same parameters as steps b) and c); and
e) Comparing the results of the first and second velocity cycles to check the integrity of the sample.

It is clarified that, step a) comprises varying the hydrostatic confining pressure between 0 and 6000 psi (41.369 MPa), in increments ΔP_{c} of 500 psi (3.447 MPa). At each increment step, the propagation velocities of compression (Vₚ) and shear (V_{S}) waves are measured. The latter are propagated along the sample's symmetry axis and obtained in two values (VS1 and VS2) because there are two internal transducers in the equipment for these waves, and they have polarization perpendicular to each other to allow the evaluation of the anisotropy of a sample.

Furthermore, once the wave velocities have been measured, the dry volumetric modulus (K_{dry}) is determined by Equation 33 and the value of the compressibility of the total volume (C_{bc}) by Equation 7. Additionally, there are also obtained the values of Poisson's ratio, Young's modulus and pore compressibility (Cₚₚ).

This step of the methodology is estimated to last approximately 3 hours. However, it will be appreciated that, depending on the type, conditions, size of the sample, among other factors, the duration of this step may vary accordingly.

Finally, it is important to note that step e) is relevant to ensure that the sample is in the elastic regime, that is, that there is no plastic deformation or rupture of the sample.

### Second Step—Dynamic Test in Saturated Sample and Constant Pore Pressure

The second step preferably comprises the steps of:
a)—Saturating the dry sample inside the pressure vessel;
b)—In a first flow operation, flowing at least 5 pore volumes through the sample;
c)—Running a first Skempton test and calculating the parameter B (by varying P_{c} and measuring the corresponding value of Pₚ);
d)—In a second flow operation, flowing at least 5 pore volumes through the sample;
e)—Running a second Skempton test and calculating the parameter B (by varying P_{c} and measuring the corresponding value of Pₚ);
f)—Checking the measured values of Skempton B from steps c) and e) and comparing the same. If the Skempton parameters B have values within ±5% of difference, the sample is considered saturated;
g)—Varying the confining pressure range, in predefined pressure increments with loading and unloading paths, and maintaining a constant pore pressure (Pₚ);
h)—Obtaining values of Vₚ and Vₛ for each of the predefined pressure increments of the confining pressure Pₚ in the cycle; and
i)—Calculating the elastic parameters Eₛₐₜ (Equation 31), vₛₐₜ (Equation 32) and Kₛₐₜ (Equation 33) for each of the predefined pressure increments of the cycle.

It is clarified that the Second Step is carried out in the same test configuration, wherein the dried sample is then saturated with deaerated water. In this step, after saturation, the Skempton coefficient values of the samples are measured with the pressure cycles, wherein the container is the pressure vessel.

Furthermore, it is clarified that dynamic tests should preferably be started by maintaining a constant pore pressure Pₚ of 1000 psi (6.895 MPa).

Once the sample is saturated and the first Skempton value B is evaluated, then the saturation process is repeated, and a second Skempton value B is calculated. If the first and second Skempton parameters B have values within ±5% of difference, the sample is considered saturated.

Once the sample saturation is complete, dynamic tests begin, maintaining a constant pore pressure (Pₚ) and varying the confining pressure (P_{c}) within a range with predefined increments (in the loading and unloading paths). Step g) comprises varying the confining pressure P_{c} from 1500 to 7000 psi (10.342 to 48.263 MPa), with increments of 500 psi (3.447 MPa), maintaining the pore pressure Pₚ at 1000 psi (6.895 MPa). At each increment step, the system maintains the confining pressure for a predetermined time and then the velocities of compression waves (Vₚ) and shear waves (V_{S}) are measured in two values: VS1 and VS2.

Once the wave velocities have been measured, the saturated volumetric compression modulus (Kₛₐₜ) is also determined (Equation 33). Additionally, Poisson's ratio and Young's modulus values are obtained.

It is also possible in this step to compare the measured saturated volumetric deformation modulus with that calculated from the Gassmann equation (Equation 38) using the volumetric deformation modulus obtained in step 1.

This step is estimated to last approximately 4.5 hours. However, it will be appreciated that, depending on the type, conditions, size of the sample, among other factors, the duration of this step may vary accordingly.

### Third Step—Static Compressibility Test on Saturated Sample

The third step preferably comprises the steps of:
1)—Simultaneously increasing the confining pressure and the a pore pressure while maintaining predefined pressure difference between the values of P_{c} and Pₚ;
2)—Maintaining the confining pressure constant;
3)—Varying the pore pressure from a predetermined maximum pressure level and then decreasing the pressure at a predefined rate to a predetermined minimum pressure value;
4)—Measuring pore volume changes ΔVₚₒᵣₑ during the test;
5)—Obtaining curves of pressure variation×volume variation of the system V_{fluid};
6)—Calculating the variation in pore volume Vₚₒᵣₑ considering the effects of the system; and
7)—Obtaining the pore compressibility Cₚₚ for the pressure variation range presented in step c).

It is clarified that the Third Step is carried out after the Second Step, and generally involves loading the confining pressure and the pore pressure simultaneously, maintaining a predefined pressure difference between them, wherein the differential pressure is at least 200 psi (1.379 MPa).

For the proposed methodology, a constant confining pressure is used. The constant confining pressure is 6000 psi (41.369 MPa). The pore pressure will be varied from a predetermined maximum pressure level and decreased at a predefined rate to a predetermined minimum pressure value. During this variation in pore pressure, the internal volume of water in the system must be maintained at a constant value. The maximum pore pressure level is 5800 psi (39.99 MPa) and the minimum pore pressure level is 1000 psi (6.895 MPa).

Furthermore, changes in pore volume (AV-pore) are measured throughout the test. The effective pressure variation curves are obtained by varying the pore volume. With these data, there is obtained the pore compressibility value Cₚₚ (Equation 10).

This step is estimated to last approximately 2 hours. However, it will be appreciated that, depending on the type, conditions, size of the sample, among other factors, the duration of this step may vary accordingly.

In a second preferred embodiment of the present invention, there is disclosed a system for carrying out the method for determining static and dynamic elastic and poroelastic parameters of rocks.

FIG. 4.2 presents a schematic illustration of this system.

The system preferably comprises:
two syringe pumps;
a pressure vessel, comprising two transducers located inside the same, for confining a sample between them;
an oscilloscope;
a pulse generator; and
a computer.

It will be appreciated that necessary electrical and mechanical connections interconnect said elements and allow communication between them, as necessary, to carry out the tests.

In this system, the sample can also be analyzed in saturated condition and with pore pressure control. One of the syringe pumps is to apply pore pressure and the other syringe pump is to apply confining pressure.

In general, the system consists of an electronic part and a hydraulic part.

The hydraulic part of the system consists of a hydraulic vessel (the core support) with two acoustic velocity transducers (AVTs), one to transmit the ultrasonic pulses and the other to receive these pulses. The sample is inserted between the emitter and receiver transducers, which are located inside the vessel.

In the electronic part, there is a function generator and a power amplifier that provide a signal to a piezoelectric transducer made of ceramic material.

In said system, it is further possible to monitor the displacement of the moving piston, providing an indication of the axial deformation of the sample.

### Examples of Embodiment

### First Step—Dynamic Test in Dry Sample Condition

Using a dry sample, the hydrostatic confining pressure is varied from 0 to 6000 psi (41.369 MPa), in increments of ΔP_{c}=500 psi (3.447 MPa) with loading and unloading paths, as disclosed in FIG. 3.1.

At each increment step, the propagation velocities of compression (Vₚ) and shear (V_{S}) waves are measured. The latter are propagated along the sample's symmetry axis and obtained in two values (VS1 and VS2) because there are two internal transducers in the equipment for these waves and they have polarization perpendicular to each other to allow the evaluation of the anisotropy of a sample.

Once the wave velocities have been measured, the dry volumetric modulus (K_{dry}) is determined by Equation 33 and the value of the compressibility of the total volume (C_{bc}) by Equation 7. Additionally, there are also obtained the values of Poisson's ratio, Young's modulus and pore compressibility (Cₚₚ).

This step of the methodology has an estimated duration of 3 hours and is presented schematically in FIG. 3.2.

It is important to note that step 5 is relevant to ensure that the sample is in the elastic regime, that is, that there is no plastic deformation or rupture of the sample.

### Second Step—Dynamic Test in Saturated Sample and Constant Pore Pressure

In the same test configuration, the dried sample is then saturated with deaerated water (the saturation process will be discussed in detail below). In this step, after the saturation, the Skempton coefficient values of the samples are measured with the pressure cycles presented in FIG. 3.3.

The process indicated in FIG. 3.3 is performed at least twice. One after the sample saturates and the first Skempton value B is evaluated, then the saturation process is repeated and a second Skempton value B is calculated. If the first and second Skempton parameters B have values within ±5% of difference, the sample is considered saturated (FIG. 3.5).

Once the sample saturation is complete, dynamic tests begin, maintaining a constant pore pressure (Pₚ) of 1000 psi (6.895 MPa) and varying the confining pressure (P_{c}) from 1500 to 7000 psi (10.342 to 48.263 MPa) with increments of 500 psi (3.447 MPa) (in the loading and unloading paths). At each increment step, the system maintains the confining pressure for 60 seconds and then the velocities of compression waves (Vₚ) and shear waves (V_{S}) are measured in two values: VS1 and VS2.

Once the wave velocities have been measured, the saturated volumetric compression modulus (Kₛₐₜ) is also determined (Equation 33). Additionally, Poisson's ratio and Young's modulus values are obtained.

It is also possible in this step to compare the measured saturated volumetric deformation modulus with that calculated from the Gassmann equation (Equation 38) using the volumetric deformation modulus obtained in step 1.

This step of the methodology has an estimated duration of 4.5 hours and is presented schematically in FIG. 3.5.

### Third Step—Static Compressibility Test on Saturated Sample

After carrying out the second step of the test, the confining pressure and the pore pressure must be loaded simultaneously, maintaining a differential pressure of at least 200 psi (1.379 MPa).

For the proposed methodology, a constant confining pressure of 6000 psi (41.369 MPa) is used. The pore pressure will be varied from a maximum level of 5800 psi (39.99 MPa) and decreased at a pre-defined rate to a value of 1000 psi (6.895 MPa), maintaining the internal volume of water in the system at a constant value, as illustrated in FIG. 3.6 and values in Table 3.1.

| Pore | Confining | Ramp | Hold | | |
|---|---|---|---|---|---|
| Pressure | Pressure | Time | Time | Rate | Rate |
| (psi) | (psi) | (s) | (s) | (psi/s) | (psi/min) |
| 5800 | 6000 | 60 | 60 | | |
| 1000 | 6000 | 6000 | 60 | −0.8 | −48 |
| 5800 | 6000 | 300 | 60 | | |

NOTE:
1 psi = 6.895 kPa

With this configuration, the alterations in pore volume (ΔVₚₒᵣₑ) are measured throughout the test. The effective pressure variation curves are obtained by varying the pore volume. With these data, there is obtained the pore compressibility value Cₚₚ (Equation 10).

This step of the methodology is presented schematically in FIG. 3.7 and has an estimated duration of 2 hours.

### Development of the Test Methodology

To verify in more detail some aspects related to the steps of the new methodology, preliminary tests were carried out to define other important aspects, with emphasis on:
a) Definition of the system time (intrinsic time) to be considered to evaluate wave propagation velocities.
b) During the loading and unloading of step 1, the level of stresses to which the samples will be subjected may be under the elastic regime.
c) To carry out the tests from step 2 onwards, it is necessary to saturate the sample inside the pressure vessel; therefore, it is necessary to use an effective saturation methodology.
d) When carrying out stage 3, it is necessary to reduce the pore pressure at a certain rate;
therefore, the appropriate pore pressure depletion rate needs to be established.

To define items b), c) and d), two samples were selected, with different lithologies—a sandstone and a carbonate; for item “d)”, a dolomite sample was also used, and a series of tests were carried out that will be presented and discussed in the next section. The samples used for these analyses have the porosity and permeability values presented in Table 4.2.

| Sample | Porosity (%) | Permeability (mD) |
|---|---|---|
| CGS-103 (Castlegate Sandstone) | 27.15 | 782 |
| IL3-103 (Indiana limestone) | 13.30 | 55 |
| SD-101 (Silurian dolomite) | 17.45 | 133 |

### System Time and Relative Position Between AVTs

Before starting dynamic tests, it is necessary to carry out an evaluation of the system time, as there is a portion of the time shift of compression and shear waves that is inherent to the system. This time must be deducted from the time shift measured with the presence of the sample. The propagation velocities (V) of elastic waves in rocks are calculated according to Equation 40 (dt=t—t₀ and l is the length of the sample).

$$V = \frac{l}{dt} \qquad ( 38 )$$

As illustrated in FIG. 3.8, the time shift of the wave in the sample is equal to the time observed on the oscilloscope minus the system time t₀ (intrinsic to the measurement system), which is the time spent by the signal in the electronics and metal heads, without any samples inserted between them [48].

In addition, to verify the best signals obtained from the two shear waves that are measured—S1 and S2—with orthogonal polarization to each other, it is also necessary to verify the best relative position of each of the AVTs (the one that generates the pulse and the one that receives the wave), to use the best possible signal quality.

For this reason, an acquisition of the P, S1 and S2 waves was carried out without sampling, that is, by placing one AVT directly against the other and, after each acquisition, a rotation was made in steps of 30° and the process repeated until one complete turn in the relative position was given. Therefore, the time shifts of P, S1 and S2 waves were measured at 0°, 30°, 60°, 90°, 120°, 150°, 180°, 210°, 240°, 270°, 300°, 330° and 360°.

Regarding the time shift of the system's P waves, it was verified after capturing the waves that the value to be used is 15.97 μs in the first peak that appears in the signal. FIG. 3.9 shows the result of the wave propagation process to check the P wave system time.

Regarding the time shift of the system's S1 waves, it was observed after capturing the waves that the value to be used is 29.74 μs in the first valley that appears in the signal. FIG. 3.10 shows the result of the wave propagation process to check the P wave system time.

Furthermore, regarding the time shift of the system's S2 waves, after capturing the waves, it was observed that the value to be used is 28.88 μs in the first peak that appears in the signal. FIG. 3.11 shows the result of the wave propagation process to check the P wave system time.

After evaluating the quality of the S1 and S2 wave signals for each relative angle between the heads, it was also verified that the best position is 0° between them (FIGS. 3.10 and 3.11). All of the work below was done using this configuration standard, as well as the system time shifts measured in this step.

### Tests to Verify Sample Behavior—Stress Level in the Elastic Regime

When applying pressure to virgin rock or even a rock recently subjected to mechanical loading (example: rock samples taken from drilling wells), it is common for internal cracks to close and grains to rearrange or adhere. After this first loading, the rock behaves linearly up to a certain stress limit within the intrinsic characteristics of the rock [11].

To verify the macroscopic integrity of the samples in tests using this method, it is proposed, as a criterion, that a dry test be carried out on each of the selected samples using two loading and unloading cycles, as shown in FIG. 3.12 (one cycle).

Such an observation aims at verifying the occurrence of any relevant damage to the sample that could affect subsequent results, even in cases in which rock strength data are not available.

The maximum level of hydrostatic pressure to which the samples were subjected was 6000 psi (41.369 MPa) in each cycle. The understanding is that, if, within these two cycles, the behavior in relation to the propagation of compression waves (which were measured at each 500 psi (3.447 MPa) variation) had been changed, it could be suggested that some damage (such as the development of small cracks) had occurred due to loading.

A sample of Castlegate sandstone (CGS-103) with dry condition was tested in two loading and unloading cycles to verify the effects of pressure on compression wave velocity values. The results of this test are presented in FIG. 3.13.

From the results presented in FIG. 3.13, it is inferred that, for the first loading of the sample, the velocities of the P waves are slightly higher than the others, but for unloading in the first cycle, as well as for loading and unloading in the second cycle, there are obtained values that are equal to each other, which indicates that their repeatability is guaranteed, which suggests that there was no relevant damage to the sample. It can be considered that, at the used pressure level, the sample behaves elastically in regions of higher confining pressures.

A sample of Indiana limestone (IL3-103) with dry condition was tested in two loading and unloading cycles to verify the effects of pressure on the values of compression wave velocities. The results of this test are presented in FIG. 3.14.

From the results presented in FIG. 3.14, it is inferred that, for the first loading of the sample, the velocities of the P waves are slightly lower, but for unloading in the first cycle, as well as for loading and unloading in the second cycle, are equal, which indicates that, for the first load, there was an accommodation of the sample and subsequently the values have their repeatability guaranteed. This suggests that there was no relevant damage to the sample, and it can be considered that at the used pressure level, the sample behaves elastically in regions of higher confining pressures.

### Tests to Verify Sample Saturation Effectiveness

Sample saturation is carried out after completion of the first test step of the method (dry test), without the need of removing the same from the pressure vessel, to save time in the process.

For such saturation, deaerated water is used to eliminate the risk of air being inserted into the system together with the water. In addition, as another way to avoid this situation, vacuum is applied to the lines, valves, vessel and the rock sample to be saturated.

Deaerated water in a minimum amount of five pore volumes of the sample is then flowed through the specimen. After carrying out this process, the Skempton coefficient value of the sample is measured. Then, again, water is drained in the minimum amount from five pore volumes of the sample and the Skempton value is measured again. If the values are of the same order of magnitude (with a maximum difference of ±5%), the sample is characterized as saturated [50].

The entire saturation process, which usually lasts around three hours, is described in more detail in the steps described below:
1. Set the confining pressure pump to 1000 psi (6.895 MPa).
2. Assemble and connect the pore pressure pump line (be careful to tighten the connection tightly).
3. Leave the valve next to the pore pressure pump closed.
4. Leave the valve next to the vessel open.
5. Place the vacuum pump hose on the valve at the bottom of the pressure vessel.
6. Turn on the vacuum pump.
7. Open the valve at the bottom of the pressure vessel.
8. Leave the vacuum pump on for 15 minutes.
9. Close the valve at the bottom of the pressure vessel.
10. Turn off the pump and remove the hose.
11. Place the water that will be used in the pore pressure pump to deaerate.
12. Refill the pore pressure pump with deaerated water.
13. Open the valve next to the pore pressure pump to bring water into the system—maintaining the valve under the container closed and set the pore pressure pump pressure to 200 psi (1.379 MPa) (3 ml/min flow limit). Wait for the fluid to fill the system and sample pores.
14. Open the valve under the container.
15. Check the maximum pump flow (should be 2 ml/min). Then, set the pore pressure pump pressure to 500 psi (3.447 MPa).
16. Alternate between slowly and quickly opening the valve at the bottom of the container to leave a flow with backpressure working and a flow of water with a lower pressure difference (the first time, to remove the honey, pass 20 ml of liquid directly).
17. Repeat the process from step 16 until at least five pore volumes of fluid flow through the system
note that the water will come out with the diluted honey (a couplant).
18. Decrease the pore pressure pump volume by approximately 3 ml. Use the flow rate of 20 ml/min.
19. Set the confining pressure pump to 4000 psi (27.579 MPa).
20. Check that the valves between the pore pressure pump and the TEMCO container are open.
21. Set the pore pump pressure to 3000 psi (20.684 MPa).
22. Wait 5 minutes in “run” mode on the pore pressure pump.
23. Set the pore pressure pump to “stop” mode.
24. Proceed with Skempton coefficient measurements (confining pressure ranging from 3500 to 4500 psi (24.132 to 31.026 MPa)) and check its stability to verify that the sample was saturated.
25. Set the pore pump pressure to 500 psi (3.447 MPa).
26. Close the valve next to the pore pressure pump.
27. Refill the pore pressure pump with deaerated water.
28. Open the valve next to the pore pressure pump.
29. Set the confining pressure pump to 1000 psi (6.895 MPa).
30. Alternate between slowly and quickly opening the valve at the bottom of the vessel to let water flow with backpressure actuation and water flow with a smaller pressure difference.
31. Repeat the process from step 30 until at least five pore volumes of fluid have flowed through the system—note that the water will come out with the diluted honey (a couplant).
32. Decrease the volume of the pore pressure pump by approximately 3 ml. Use the flow rate of 20 ml/min.
33. Set the confining pressure pump to 4000 psi (27.579 MPa).
34. Check that the valves between the pore pressure pump and the TEMCO container are open.
35. Set the pore pressure pump to 3000 psi (20.684 MPa).
36. Wait 5 minutes in “run” mode on the pore pressure pump.
37. Place the pore pressure pump in “stop” mode.
38. Proceed with Skempton coefficient measurements (confining pressure ranging from 3500 to 4500 psi (24.132 to 31.026 MPa)) and check its stability to verify that the sample was saturated.
39. Compare the values obtained in steps 24 and 38. If they are similar, consider the saturation process carried out.

After applying the saturation process described above to two different samples, the results of the measurements of the Skempton coefficient values will be described.

For the sandstone sample CGS-103, FIGS. 3.15 and 3.16 present the applied confining pressure values and the resulting measured pore pressure values. With these data, the following values were achieved:
B=0.411 after the first flow of 5 pore volumes (FIG. 3.15)
B=0.401 after second flow of 5 pore volumes (FIG. 3.17)

For the IL3-103 carbonate sample, FIGS. 3.17 and 3.18 present the applied confining pressure values and the resulting measured pore pressure values.

With these data, the following values were achieved in:
B=0.290 after the first flow of 5 pore volumes (FIG. 3.17).
B=0.280 after a second flow of 5 pore volumes (FIG. 3.18).

Because the Skempton coefficient values were remarkably close after flowing five pore volumes twice through each sample, it can be considered to be saturated when using the process described in this section. Such a process is used in the methodology and previously applied to conduct the tests in Steps 2 and 3.

It is important to mention that the saturation process used is valid for homogeneous samples and with adequate permeability and porosity values.

### Tests to Determine the Rate of Reduction in Pore Pressure

Initially, it was considered using a pore pressure reduction from 5800 to 200 psi (39.99 to 1.379 MPa). However, it is necessary to define the reduction rate of this pore pressure to be used in the tests to obtain sufficiently reliable results within an optimized execution time.

In previous tests carried out [16], a pore pressure reduction rate of 0.27 psi/s (1.86 kPa/s) was used, which was quite sufficient in that context, but the author emphasized that such a situation would not be appropriate in some cases.

To solve this issue, some tests were carried out with three samples from different lithologies, varying the values of the pore pressure reduction rates. The results will be presented in this section.

To verify the influence of the pore pressure decrease rate on the sandstone sample CGS-103, a series of tests were carried out on the same sample with different rates. The pore pressure pump presented a water volume of around 10 ml in all tests. The pore pressure ranged from a maximum pressure of 5800 psi (39.99 MPa) to a minimum of 200 psi (1.379 MPa). The confining pressure was set at 6000 psi (41.369 MPa). The tests were carried out according to Table 3.3.

| Pore pressure | Rate (psi/s) | Test duration |
|---|---|---|
| reduction time (s) | (×6.895 kPa/s) | (in hours) |
| 600 | −9.66 | 0.2 |
| 1200 | −4.83 | 0.3 |
| 1800 | −3.22 | 0.5 |
| 2400 | −2.41 | 0.7 |
| 3600 | −1.61 | 1.0 |
| 4800 | −1.20 | 1.3 |

By analyzing the data for each of the rates used, it was found that for the Cₚₚ values obtained there was a difference of ±1% between them, using the minimum and maximum rates evaluated. This is considered adequate and has no impact on the values obtained.

In addition, as can be seen in FIG. 3.19, for effective pressures above 5000 psi (34.474 MPa), there is a behavior of compressibility values that do not actually represent what is happening in the sample, as such values can suggest collapse of pores in the sample and, as seen previously, for pressure differences of up to 6000 psi (41.369 MPa), such a situation is not expected. This distortion in values is due to the limitation of the pump, as this equipment controls the pressure by volume variation, and when it needs to stop at a certain pressure, the volume variations begin to decrease to reach the necessary pressure. To solve this limitation, a pressure difference limit (P_{c}−Pₚ) of at least 1000 psi (6.895 MPa) was stipulated.

FIG. 3.20 presents pressure difference values (P_{c}−Pₚ) within at least 1000 psi (6.895 MPa) and represents the compatible behavior of the sample, which suggests that, within the limits of the equipment used, such a restriction of minimum effective pressure value must be considered.

To verify the influence of the pore pressure decrease rate in the sample IL3-103, a series of tests were performed on this sample with different rates. In the pore pressure pump, a water volume of around 10 ml was maintained in all tests. The pore pressure ranged from a maximum pressure of 5800 psi (39.99 MPa) to a minimum of 200 psi (1.379 MPa). The confining pressure was set at 6000 psi (41.369 MPa). The tests were carried out according to Table 3.4.

| Pore pressure | Rate (psi/s) | Test duration |
|---|---|---|
| reduction time (s) | (×6.895 kPa/s) | (in hours) |
| 600 | −9.66 | 0.2 |
| 1200 | −4.83 | 0.3 |
| 1800 | −3.22 | 0.5 |
| 2400 | −2.41 | 0.7 |
| 3600 | −1.61 | 1.0 |
| 4800 | −1.20 | 1.3 |
| 7200 | −0.80 | 2.0 |
| 9600 | −0.60 | 2.7 |
| 21500 | −0.27 | 6.0 |

With the data obtained for each of the rates, it was analyzed that, for the Cₚₚ values achieved, there was a difference of ±5% between them, using the minimum and maximum rates evaluated. This is considered adequate and has no impact on the quality of the results. In addition, an evaluation was made for the reduction rate of 0.25 psi/s (1.724 kPa/s) used by Newman [16]. Such a rate does not change the general appearance of the obtained curves in terms of shape and values.

In addition, as can be seen in FIG. 3.21, for effective pressures above 5000 psi (34.474 MPa), there is a behavior of compressibility values that do not actually represent what is happening in the sample. Such values may suggest damage to the sample and, as seen previously, for pressure differences of up to 6000 psi (41.369 MPa), this situation is not expected as occurred in the sandstone sample. The distortion in values is a limitation of the pump that applies pore pressure, as discussed previously.

FIG. 3.22 presents pressure difference values (P_{c}−Pₚ) of at least 1000 psi (6.895 MPa) and represents the compatible behavior of the sample, which suggests that, within the limits of the equipment, such a restriction of minimum effective pressure value must be considered.

By taking the presented results into account, it was considered that the minimum pressure difference limit is 1000 psi (6.895 MPa) for the method (FIG. 3.6). Even with the results obtained in these two samples, a variation rate of 0.8 psi/s (5.516 kPa/s) (50 psi/min (344.74 kPa/min)) was adopted, as a standard as it is sufficiently accurate.

Table 3.1 above presents the protocol established for Step 3.

To complement the influence of the depletion rate, a test was also carried out with a dolomite sample (SD-101) using the already suggested protocol (pore pressure reduction rate of 0.8 psi/s (5.516 kPa/s)) and with depletion rate of 0.32 psi/s (2.206 kPa/s) (FIG. 3.23).

For this sample, the biggest differences occurred at low effective pressures, a region in which there is normally no relevant application of compressibility values in engineering studies. It is important to note that between 2000 and 5000 psi (13.79 and 34.474 MPa) of effective pressure the values are close, within the estimated margin of error for this process (±5.00%).

### Experimental Configuration

Next, the system used for laboratory tests will be presented with the methodology and calibration of the set carried out before evaluating the selected samples.

### Equipment Used in the Experimental Methodology

The experimental configuration (FIG. 4.1) consists of two syringe pumps: one to apply pore pressure (on the left) and one to apply confining pressure (on the right); a pressure vessel where a sample is tested (in the center); an oscilloscope (on the front), a pulse generator (on the front) and the electrical and mechanical connections necessary to run the tests.

FIG. 4.2 illustrates a schematic of the experimental configuration used to run elastic wave velocity tests on rocks. It consists of a cylindrical stainless steel vessel with fluid reservoirs that allow different radial and axial pressure conditions to be imposed on the samples (in the present case, the chambers were connected to allow the application of hydrostatic pressure using just one pump).

The elastic velocity measurement system used in this analysis consists of an electronic part and a hydraulic part, as illustrated in FIG. 4.2.

The hydraulic part of the system consists of a hydraulic vessel with two acoustic velocity transducers (AVTs), one to transmit the ultrasonic pulses and the other to receive these pulses. The sample is inserted between the emitter and receiver transducers, which are located inside the container.

In the electronics, there is a function generator and a power amplifier that provide a known signal to a piezoelectric transducer made of ceramic material. This method follows the standards established by ASTM D2845-08 [51].

With said system, it is possible to monitor the displacement of the moving piston—giving an indication of the axial deformation of the sample. A dimensional limitation of this equipment is the maximum sample length (50 mm) and the diameter of 1.5″ (38.1 mm).

Core Support with Acoustic Velocity Transducers (AVTs)

The core support used has two acoustic velocity transducers (AVTs), one at the top and one at the bottom to send and receive the ultrasonic pulses to run dynamic tests on a sample. The maximum operating pressure of the equipment is 10,000 psi (68.946 MPa). FIG. 4.3 shows an overview of this equipment.

At the top is located the piston that can move (when pressure is applied to the core support), as well as the wires that connect the AVTs to the pulse generator. At the bottom is a fixed part of the chamber, as well as the wires that connect the AVTs to the pulse generator.

FIGS. 4.4A and 4.4B illustrate the configuration for mounting the specimen in the vessel, using nitrile rubber material as a lining material to isolate the sample from the confining fluid. A shear gel was used as a coupling material, inserted between the top and bottom of the sample and the AVTs to improve the output signal [48].

Inside the vessel, there are two AVTs, one that inserts the ultrasonic pulse into the sample and another that receives the pulse that traveled through the sample, as disclosed in FIGS. 4.5A and 4.5B.

The pulse receiving transducer is on the left (it is the part that can move—piston—when applying pressure to the system), and the pulse transmitting transducer is on the right (this part does not move).

Schematically, the hydraulic system is shown in FIG. 4.6.

The axial pump is connected to the fluid chamber using a line and valve (V3). The pore pressure pump is connected to the vessel through a line and valve (V5), and valve V1 is at the end. Valve V4 is always closed because there is no need to use this part.

For the tests, valve V2 (FIG. 4.6) was always kept open to connect the axial and radial pressure chambers, to subject the sample to the hydrostatic confining pressure as stipulated.

Inside the container, the sample is mounted and its upper and lower contact with the AVTs must be guaranteed. In addition, it is necessary to use a rubber material on the sample to avoid contact of the fluid (which applies the confining pressure) with the sample.

The transducer (FIGS. 4.7A and 4.7B) transforms the electronic signal into a mechanical, compression or shear vibration, as appropriate. After traveling through the rock sample, the mechanical vibration is received by another transducer, which converts the same into an electrical signal.

It is important to mention that before starting tests using this equipment, it is necessary to carry out some tests to measure the system time. With this evaluation, all errors in the mechanical/electronic set are already considered in this step.

### Oscilloscope and Ultrasonic Pulse Generator

To generate the necessary pulses, an ultrasonic pulse generator from the manufacturer Olympus is preferably used—model 5058PR—FIGS. 4.8A and 4.8B. It was developed specifically for this purpose and used with the aim of attenuating the signal and offering greater accuracy of results.

The electronic signal from the receiver is analyzed on an oscilloscope from Agilent Technologies, model DSO7012B. The signals can be acquired and stored locally using the automated system.

### Syringe Pumps, Lines and Valves

Two syringe pumps, preferably from Teledyne ISCO, were used to deliver pressurized fluid to confine the sample during testing, as illustrated in FIGS. 4.9A and 4.9B. Both pumps have high-precision pressure and flow rate controls as their main feature and are controlled manually or by automated systems.

The pump used to apply the confining pressure is preferably the 100DX model, as it has a maximum pressure limit of 10,000 psi (68.946 MPa) and a maximum internal volume of 102.93 ml. The error in flow rate values is of the order of ±0.3% and in pressure values of ±0.5%, according to the manufacturer's manual.

Next, the pump used to apply pore pressure is preferably the 260D model, as illustrated in FIG. 4.10B, which has a maximum pressure limit of 7500 psi (51.711 MPa) and a maximum internal volume of 266.05 ml in the equipment. The error in flow rate values is of the order of ±0.5% and in pressure values of ±0.5%, according to the manufacturer's manual.

The lines and valves that make up the system are compatible with the maximum pressures and have an internal diameter of 1 mm in order to reduce the overall volume of fluid storage in the system. Both are made of high mechanical strength stainless steel. Furthermore, the valves have high quality in terms of tightness and precision in closing and opening modes.

### Automated System

The system is automated, allowing control of the pressures to be applied to the sample, through the connection and control of the pumps (FIG. 4.11).

The same system is connected to the pulse generator and the oscilloscope to allow the collection of data on the wave propagation velocities, as well as the prior programming of the stress trajectory to be applied. The waveforms are stored on a computer for later analysis.

Preferably, the data obtained by the elements/equipment is stored on a computer storage medium.

The storage medium above may include any medium that can store the data, such as a non-transitory readable storage medium, a flash memory, a removable hard disk, a read-only memory, a random access memory, a magnetic disk, or an optical disc.

### System Calibration

Using the proposed method, the measurement of pore compressibility Cₚₚ is not obtained directly by applying the hydrostatic pressure or varying the pore pressure. The idea of calibration is to consider the effect of the variation in the volume of the system (valves, piston, pipes) and the water (or other fluid) in the system to have the most correct value of variation in the volume of the pores (ΔVₚₒᵣₑ), to calculate Cₚₚ (Equation 41) [3].

$${C}_{p p} = \frac{1}{{V}_{p o r e}^{i}} {( \frac{Δ {V}_{pore}}{Δ {P}_{p}} )}_{{P}_{c}} \qquad ( 39 )$$

Some authors have already pointed out the importance and difficulties inherent in carrying out tests to achieve these Cₚₚ values [53].

A prominent study focused on this issue was carried out to establish the influence of the system elements involved in the tests, indicating the relevance of corrections to the values obtained in order to establish the most correct value, discounting the influence of the system itself [54].

Going a little further, it is possible to indicate the errors related to the measurements due to the influence of the system in obtaining the Cₚₚ, which would typically vary from 7 to 23%, depending on the compressibility and porosity of the sample involved [55].

To acquire the estimated value of the variation in pore volume (which is the quantity necessary to calculate the Cₚₚ), in principle, calibration tests can be carried out, for example, using a rigid steel sample, to determine how much variation in volume the equipment and fluid will suffer in the test [53]. Furthermore, in the context of this invention, obtaining the effects of the fluid—equipment (FIG. 4.12)—sample will be called calibration.

Observing FIG. 4.12, the relevant equipment to be considered in calibration are the pump that applies internal pressure (on the left), the valves (on the right and left), the lines (at the top) and the pressure vessel (in the center).

Due to the difficulties mentioned in obtaining the value of the pore volume variation—when the pore pressure is varied to obtain the Cₚₚ value—it is necessary, when using a fluid of known compressibility, to verify the effects of the equipment to be used on the tests of this methodology to obtain as close as possible the exact value of the variation in pore volume.

To carry out the calibration of the experimental arrangements that were used in compressibility tests with the proposed methodology, the approximation indicated by Jaeger (Equation 42) was used to estimate the variation in pore volume [53].

$$Δ {V}_{fluid} = Δ {V}_{p o r e} + Δ {V}_{e q u ipment} \qquad ( 40 )$$

Where:
ΔV_{fluid} —Variation in volume of the fluid present in the system (pump, lines, sample pores).
ΔVₚₒᵣₑ —Variation in the volume of the sample pores.
ΔV_{equipment} —Variation in the volume of the equipment (pump, lines, valves and pressure vessel).

In the static compressibility tests of the new proposed methodology, the measurement obtained directly is ΔV_{fluid}, that read in the pump that controls the pore pressure.

System calibration was performed using deaerated water as the internal fluid for sample saturation and to apply pressure to the samples. The calibration with water was carried out in two steps as shown below:
First step (AVT-AVT): placing the system without any sample, that is, placing the two AVTs touching each other, to obtain a function to correct the measured values, when running the test to obtain the pore compressibility (Cₚₚ) of a sample
Second step: using an aluminum cylinder (with a hole to simulate porosity) as a sample to check the behavior of the entire system with a rigid sample and checking the results using the calibration function obtained in the first step.

In these two calibration steps, the following parameters were used:
Confining pressure (hydrostatic): 6000 psi (41.369 MPa)
Pore pressure: initial equal to 5800 psi (39.99 MPa)/final equal to 200 psi (1.379 MPa)
Pore pressure decrease rate: 10 psi/s (68.95 kPa/s)
Volume of fluid in the system (initial): 30 ml, 20 ml, 15 ml, 10 ml, 5 ml and 1 ml.

These tests were carried out with the aim of verifying the effect of varying the amount of water in the set, which influences the compressibility of the system on the results, to more adequately estimate the volume variation that occurs specifically in the pores of a rock sample, as described above.

### Sample-Free Calibration Results—First Step (AVT-AVT)

Volume variations were measured as a function of the decrease in pore pressure for different internal volumes of water in the pore pressure pump (30 ml, 20 ml, 15 ml, 10 ml, 5 ml and 1 ml).

FIG. 4.13 shows a surface correlating the initial pump volume, pump pressure, and volume change. The initial volume axis is the value of the fluid present in the pump at the beginning of the measurement. The volume variation axis represents the changes in volume when pore pressure varies. The pore pressure axis indicates the variation of this parameter.

More succinctly, FIG. 4.14 shows where the curve with the lowest volume variation values corresponds to 1 ml in the pump, and the curve with the highest volume variation values corresponds to 30 ml.

With the data collected point by point for each volume, a regression was performed in order to obtain the correction function to be used in the volume values measured in the tests. The program used was MATLAB and the “Poly22” adjustment function. Such a function performs a linear regression using the least squares method, to fit a second degree polynomial surface two for pore pressure (Pₚ) and a second degree one for the system volume values (V_{system}) For this case, the results are summarized in Equation 43, where Pₚ is the pore pressure (psi—1 psi=6.895 kPa), V_{system} is the volume read in the pore pressure pump (ml) and “pn” are the coefficients of this function (Table 4.1, presented below).

$$f ( {P}_{p} , {V}_{system} ) = p 00 + p 10 . {P}_{p} + p 01 . {V}_{system} + p 20 . {P}_{p}^{2} + p 11 . {P}_{p} . {V}_{system} + p 2. {V}_{system}^{2} \qquad ( 41 )$$

The polynomial coefficients are presented in Table 4.1 with 95% confidence (lower and upper limits in parentheses).

| Coefficient | Value | Lower and upper limits |
|---|---|---|
| p00 | 1,032 | (1,031; 1,032) |
| p10 | −0.0002013 | (−0.0002017; −0.0002009) |
| p01 | 0.01838 | (0.01832; 0.01844) |
| p20 | 4,341e−09 | (4,281e−09; 4,401e−09) |
| p11 | −3.248e−06 | (−3,257e−06; −3,239e−06) |
| p02 | 8,541e−06 | (6,973e−06; 1,011e−05) |

It's worth remembering that:

$$Δ {V}_{fluid} = Δ {V}_{p o r e} + Δ {V}_{e q u ipment} \qquad ( 42 )$$

This is reorganized as follows:

$$Δ {V}_{pore} = Δ {V}_{fluid} - Δ {V}_{e q u ipment} \qquad ( 43 )$$

Where:
ΔV_{fluid} —is obtained directly from the volumes recorded in the pore pressure pump.
ΔV_{equipment} —represents the influence of equipment (valves, pump piston, pipes) on the volume variation (Eq. 45). This value is calculated for each pair of pressure and volume in the system using the system calibration data from Equation 43.

$$Δ {V}_{e q u i p m ent} = f ( {P}_{p} , {V}_{s ystem} ) \qquad ( 44 )$$

It is important to highlight that the proposed correction cannot be in the order of magnitude of the sample deformations, as it may bring distortions to the process that will not be supported by this methodology.

Test Carried Out with an Aluminum Sample—Second Step

To verify the behavior of the system with a sample of known behavior and rigidity as the system, another test was carried out using an aluminum cylinder with a hole as a sample (FIGS. 4.15A and 4.15B). This was done to simulate the effect of pore pressure on the same. This sample has some properties summarized in Table 4.2.

| Mass | 116.96 | g |
|---|---|---|
| Length | 39.82 | mm |
| Outer diameter | 38.18 | mm |
| Hole diameter | 9.93 | mm |
| Porosity | 7.1% | |
| Density | 2.71 | g/cm³ |

Aluminum Cₚₚ measurements were made using two volumes in the system: 2 ml and 10 ml. The values were adjusted by Equation 43, obtained in the calibration process.

FIG. 4.16 presents Cₚₚ directly measured results and the results adjusted with calibration. The values acquired with the correction are called adjusted values.

As a complement, a numerical analysis of the same sample was carried out under the same test conditions. Similar values (Cₚₚ=2.76×10⁻⁷ psi⁻¹) (1 psi=6.895 kPa) were obtained through the results of the corrected test (average values) and numerical analysis (carried out using the finite element method, preferably in the ABAQUS program).

### Rock and Fluid Samples

Next, there is the selection and preparation of the samples that were used for the tests using the proposed methodology. The petrographic, mineralogical and volumetric characterizations of the selected samples are also indicated.

The volumetric compressibility modulus of the sample grains and the fluid (water) used in the tests will also be presented.

### Sample Selection and Preparation

To have some representative lithologies, outcrop rock samples were selected from the supplier Kocurek Industries (https://kocurekindustries.com).

Sandstone and carbonate samples were available for testing with different porosities and Young's modulus (FIG. 5.1). The elastic moduli presented in FIG. 5.1 were obtained from dynamic tests carried out in the CENPES laboratory.

From these available outcrop samples, the selection was made to represent sets of the same lithology with different porosities and permeabilities, in the case of sandstones, and with different values of unconfined compressive strength (UCS) in the case of carbonates (FIG. 5.2).

The outcrop samples selected for testing and the UCS values for each are presented in Table 5.1.

| | UCS (psi − 1 |
|---|---|
| Sample | psi = 6.895 kPa) |
| Castlegate Sandstone (CGS) | 2000 to 2500 |
| Bentheimer Sandstone (BES) | 3500 to 4500 |
| Indiana Limestone (IL3) | 4000 to 5000 |
| Silurian Dolomite (SD) | 10,000 to 12,000 |

After selecting the samples that are distributed in cylinders 10″ (254 mm) long and 1.5″ (38.1 mm) in diameter, they were all separated (FIG. 5.2) and then cut into the necessary dimensions (1.5″×2.0″ (38.1 mm×50.8 mm)) to the tests (FIG. 5.3) following the recommendations of McPhee et al. [57].

After cutting, the flat faces of each sample were mechanically sanded for level and checked for parallelism with the opposing faces. Then, all samples were subjected to the cleaning process to remove salts.

All of these samples, after cutting, had their masses and geometry measured. They were also prepared to guarantee parallelism between the faces of the sample and subsequently subjected to routine petrophysical tests to better characterize the porosity, permeability and specific mass of the solids in the samples.

### Petrographic and Mineralogical Characterization

For the mineralogical characterization of the samples, slides were prepared for each of the four lithologies for petrographic analysis. In addition, X-ray Diffractometry (XRD) tests were performed to verify the minerals in each sample using leftover sections when the samples were prepared.

### Petrographic Description and Visual Analysis

The samples were described to characterize their textures and structures. The method adopted was optical petrography using slides for each lithology of interest.

Below, there will be presented the results of these evaluations for each lithology.

Castlegate sandstone is a fine to medium, well sorted, sub-rounded, massive sandstone, having intergranular, intragranular and moldic porosity, as illustrated in FIGS. 5.9A and 5.9B.

Bentheimer Sandstone is fine to medium well-sorted, sub-rounded, massive sandstone, having types of intergranular and intragranular porosity, as illustrated in FIGS. 5.10A and 5.10B.

Indiana limestone is a very coarse and poorly sorted bioclastic grain (grains vary from fine to grainy sand). The contacts between the grains are punctual and flat, indicating moderate compaction. There is the presence of dissolution in some grains generating intragranular porosity, as illustrated in FIGS. 5.11A and 5.11B.

Silurian dolomite is a medium to coarse dolomite. Dolomite crystals are euhedral to subhedral and occur in a dense mosaic-like arrangement, with portions of less dense arrangement. The presence of stylolite indicates chemical compaction. Some vugs have rounded shapes, possibly originating from the dissolution of the grains. It presents intercrystalline and vugular porosity, as illustrated in FIGS. 5.12A and 5.12B.

### Mineralogical Composition by X-Ray Diffractometry (XRD)

Tests were conducted to check the minerals in each sample using leftover cuts when the samples were prepared.

These remaining sections were subjected to an analysis by X-ray diffractometry (XRD). This methodology is based on the interaction of electromagnetic waves at the frequency of X-rays with the crystalline rock materials, thus being an important tool for the mineralogical characterization of samples [58].

The results of these analyses are presented in Table 5.2.

| Sample | CDA | CMG | DOL | QTZ | A + M | CAL | KFD |
|---|---|---|---|---|---|---|---|
| Silurian Dolomite | 9 | | 91 | 0 | | | |
| (SD) | | | | | | | |
| Castlegate (CGS) | | | | 86 | 6 | | 8 |
| Indiana Limestone | | 34 | | | | 66 | |
| (IL3) | | | | | | | |
| Bentheimer (BES) | | | | 95 | | | 5 |

CDA = Ca-Dolomite/Ankerite;
CMG = Magnesian Calcite;
DOL = Dolomite;
QTZ = Quartz;
A + M = Clay minerals + phyllosilicates;
CAL = Calcite;
KFD = K-feldspar;
TR = Trace (<1%)

### Volumetric Characterization

For the quantitative and qualitative characterization of the values associated with the sample volumes—both the rock matrix and its pores—some tests were carried out with different purposes. The main characteristics of these tests and their results will be presented below.

### Basic Petrophysics

The samples were subjected to tests to obtain porosity, permeability and specific mass of solids, using the methods found in the API 40 standard (API, 1998), in particular the method described in item 5.3.2.2.1 to obtain porosity and in item 6.3.1.1.1.2. to obtain permeability (Nitrogen Gas).

The results are presented in Table 5.3.

| | | | | | | | Specific | |
|---|---|---|---|---|---|---|---|---|
| | | | | Confining | Absolute | Effective | mass of | Porous |
| Sample | Diameter | Length | Weight | pressure | permeability | porosity | solids | Volume |
| Code | (cm) | (cm) | (g) | (psi) | (mD) | (%) | (g/cm³) | (cm³) |
| BES-101 | 3.79 | 4.87 | 110.97 | 1000 | 2420 | 23.22 | 2.64 | 12.71 |
| BES-102 | 3.79 | 4.76 | 108.79 | 1000 | 2420 | 22.99 | 2.64 | 12.30 |
| BES-103 | 3.79 | 4.80 | 109.66 | 1000 | 2270 | 22.88 | 2.63 | 12.33 |
| CGS-101 | 3.69 | 4.86 | 100.08 | 1000 | 676 | 26.78 | 2.63 | 13.88 |
| CGS-102 | 3.69 | 4.80 | 98.22 | 1000 | 772 | 26.95 | 2.63 | 13.74 |
| CGS-103 | 3.68 | 4.73 | 96.21 | 1000 | 782 | 27.13 | 2.63 | 13.59 |
| CGS-106 | 3.75 | 4.89 | 100.24 | 1000 | 911 | 27.00 | 2.62 | 14.17 |
| IL3-101 | 3.75 | 4.84 | 123.16 | 1000 | 68 | 13.36 | 2.66 | 7.11 |
| IL3-102 | 3.74 | 4.88 | 123.4 | 1000 | 71 | 13.56 | 2.66 | 7.25 |
| IL3-103 | 3.75 | 4.80 | 122.33 | 1000 | 55 | 13.26 | 2.67 | 7.00 |
| IL3-106 | 3.74 | 4.93 | 120.34 | 1000 | 146 | 16.17 | 2.65 | 8.76 |
| SD-101 | 3.74 | 4.73 | 120.67 | 1000 | 133 | 17.44 | 2.82 | 9.03 |
| SD-102 | 3.74 | 4.81 | 120.78 | 1000 | 257 | 18.86 | 2.82 | 9.95 |
| SD-103 | 3.74 | 4.85 | 121.76 | 1000 | 256 | 18.74 | 2.81 | 9.96 |
| SD-106 | 3.74 | 4.97 | 123.03 | 1000 | 747 | 18.64 | 2.78 | 10.15 |

NOTE:
1000 psi = 6.895 MPa

### Microtomography

For microtomographic analysis of the samples, a microtomography was used, preferably from GE/Phoenix (FIG. 5.13). The basic principle of this equipment is the emission of X-rays that flow through the sample. The obtained results refer to the attenuation of X-rays during propagation in the sample, between the source and the receivers [48].

In order to reconstruct the solid in three dimensions, the sample is rotated around its longitudinal axis, maintaining the source and receivers fixed. This process is called tomographic reconstruction [48].

The images are processed for each lithology, with visual indication of the shape of the pores, as well as the internal characteristics of these pores of the samples used in the process.

It is important to note that the result obtained through the reconstruction process is an image that represents the attenuation coefficients of the rock in 3D. In the present invention, these values are available in gray scale and are shown in mosaics representing the XY and XZ sections of the sample image.

The X-ray attenuation coefficients of a material depend on the energy of the used photon beam, the density and the effective atomic number of the material. Darker regions in the images are related to pores, regions with microporosity or minerals with low density or atomic number. The lighter regions show phases without porosity, high density minerals or high atomic number.

FIG. 5.14 presents some sections of the tested samples, where the shapes and distribution of their pores can be qualitatively visualized.

FIGS. 5.15 and 5.18 show different cross and longitudinal sections for each of the lithologies used in the tests. It is possible to visualize the shapes and distribution of their pores in a qualitative way.

### Characteristics of Pores

The mercury injection test was used to determine some pore characteristics, such as the radii of the pore throats and their distribution in the sample. To establish the distribution of the pore throats [60], the method basically consists of applying increasing pressures (up to 60,000 psi (413.685 MPa)) to the mercury phase, forcing it into a clean, dry rock fragment. The mercury intrusion pressure (non-wetting phase) is inversely proportional to the pore throat radii.

FIGS. 5.20 and 5.21 present the results for the Castlegate Sandstone sample. It can be observed that there is the presence of microporosity for this lithology.

FIGS. 5.22 and 5.23 present the results for the Bentheimer sandstone sample and, for this lithology, there is no presence of microporosity, confirming what was indicated by the analysis of the petrographic slides (FIG. 5.10).

FIGS. 5.24 and 5.25 present the results for the Indiana Limestone sample. It can be observed that there is the presence of microporosity for this lithology.

The results for the Silurian dolomite carbonate sample are presented in FIGS. 5.26 and 5.27, where it is observed that there is the presence of microporosity, but at a lower value than the Indian Limestone sample.

### Volumetric Compressibility Modulus of Sample Grains

With the proportion of each mineral present in the rock matrix of all samples measured by XRD (Table 5.1) and the K values of the minerals (Table 5.4), the volumetric compression moduli (Table 5.5) of the grains (K_{g}) were obtained for each lithology using the Voigt-Reuss-Hill average (Equation 36) [25]. This average is a representative value because it uses the K average values of the upper (Voigt) and lower (Hill) limits [25].

TABLE 5.4

| | Bulk modulus K | Shear modulus μ | | | Poisson's | |
|---|---|---|---|---|---|---|
| Mineral | (GPa) | (GPa) | Vₚ (km/s) | Vₛ (km/s) | Ratio | Density (g/cm³) |
| Calcite (CAL) | 70 ± 7 | 30 ± 3 | 6.45 ± 0.24 | 3.34 ± 0.10 | 0.31 ± 0.01 | 2.71 |
| Dolomite (DOL) | 83 ± 12 | 48 ± 4 | 7.14 ± 0.20 | 4.10 ± 0.13 | 0.25 ± 0.05 | 2.87 ± 0.01 |
| Quartz (QTZ) | 37 ± 0.5 | 45 ± 0.2 | 6.05 ± 0.01 | 4.12 ± 0.03 | 0.07 ± 0.01 | 2.65 |
| Anhydrite (AND) | 59 ± 3 | 31 ± 3 | 5.90 ± 0.25 | 3.25 ± 0.15 | 0.27 ± 0.01 | 2.97 ± 0.01 |
| Barite (BAR) | 57 ± 3 | 23 ± 0.8 | 4.47 ± 0.03 | 4.98 ± 0.20 | 0.32 ± 0.01 | 4.46 ± 0.04 |
| Pyrite (PIR) | 143 ± 05 | 121 ± 12 | 7.90 ± 0.20 | 4.39 ± 0.11 | 0.17 ± 0.02 | 4.87 ± 0.06 |
| Shale (ARG) | from 1.5 to 35 | from 1.4 to 12 | from 1.5 to 5.0 | from 1.5 to 2.6 | from 0.20 to 0.35 | from 2.13 to 2.75 |
| Silica | 25 | 25 | 4.98 | 3.26 | 0.12 | 2.35 |
| Feldspar - rich in K | 48 | 24 | 5.59 | 3.06 | 0.29 | 2.56 |

TABLE 5.5

| Sample | Kg (GPa) |
|---|---|
| Silurian Dolomite (SD) | 83.00 |
| Castlegate Sandstone (CGS) | 36.34 |
| Indiana Limestone (IL3) | 70.00 |
| Bentheimer Sandstone (BES) | 37.49 |

### Fluid Used for Saturation and Application of Pore Pressure in Tests

The fluids in the pores of a rock influence its properties, as they change the density, wave propagation velocity, volumetric compressibility modulus, among others [62].

For the purposes of the present invention, the fluid used was deaerated water, as it has low compressibility when compared to oil, for example, and this characteristic is relevant to obtain the best results for system calibration.

The acoustic parameters of the water, necessary for fluid replacement, were obtained using an internally developed program, and the calculations of the parameters were essentially based on the work of Batzle [62], and are illustrated in FIG. 5.28.

The considered water compressibility modulus (K_{fluid}) was 2.51 GPa (FIG. 5.28). This value was obtained at a temperature of 20° C. and a hydrostatic pressure level of 39.99 MPa (5800 psi).

### Test Results Using the Methodology

The test results using the test methodology of the present invention will be presented below. These tests were performed on 13 different samples with 4 different lithologies, as previously described.

To recap, the first and second steps (dynamic tests) are performed to measure the values of the P and S wave velocities. With these values, the elastic moduli are calculated.

The equipment measures shear wave velocities with orthogonal polarization, generating two different velocities (VS1 and VS2).

The methodology involves saturating the sample inside the vessel (called “first flow”) and, after carrying out the tests to obtain the first value of the Skempton coefficient (B), the saturation process is repeated (“second flow”) to obtain a new value B later. If these coefficients have values close to each other, the sample is considered saturated.

It is also important to highlight that, for fluid replacement using Gassmann, the grain compressibility modulus values were obtained using the Voigt-Reus-Hill average (Table 5.5). S wave velocity values after saturation may contain inaccuracy due to the difficulty in identifying events in the wave propagation signals (low signal-to-noise ratio), and K_{fluid} was calculated.

The third step (static test) consists of controlling the pore pressure variation, maintaining the confining pressure constant and obtaining the corresponding pore volume variation values to calculate the Cₚₚ value for each pressure level (Equation 12).

### Castlegate Sandstone (CGS)

The test results using the proposed methodology for the three selected Castlegate sandstone samples (CGS-101, CGS-102 and CGS-106) are presented below.

FIGS. 6.1 to 6.3 indicate the behavior of compressive wave velocities for each of the two loading and unloading cycles of the dry sample.

As can be seen, for higher levels of confining pressure, the velocity values behave very close, within the expected error (1%). Furthermore, based on the shape of the curve, it can be said that there was no damage to the samples at the stress levels applied in the tests, when comparing the values obtained in cycles 1 and 2.

Another important topic concerns the values of compression and shear wave velocities obtained for each dry sample; such results are shown in FIGS. 6.4 to 6.6.

The shear wave velocity values (FIGS. 6.4 to 6.6) present small differences between them. If this problem were in larger proportions, it could indicate that the material would have anisotropy behavior, but this is not the case. In addition, as this behavior is not expected with this material, VS1 or VS2 can be used to calculate the elastic parameters.

Table 6.1 presents a compilation of the elastic and poroelastic moduli values obtained from the wave propagation velocity values measured for each sample at a confining pressure equal to 6000 psi (41.369 MPa).

| | | | Dynamic tests | | | | |
|---|---|---|---|---|---|---|---|
| | Sample data | | Dry (Stage 1) | | | | |
| | ϕ | k | E | | K_{dry} | Cₚₚ | |
| Sample | (%) | (mD) | (GPa) | ν | (GPa) | (GPa⁻¹) | α |
| CGS-101 | 26.78 | 676 | 17.46 | 0.24 | 11.10 | 0.2061 | 0.694 |
| CGS-102 | 26.95 | 772 | 17.20 | 0.23 | 10.71 | 0.2168 | 0.705 |
| CGS-106 | 27.0 | 911 | 17.41 | 0.23 | 10.67 | 0.2176 | 0.706 |

The Cₚₚ values presented in Table 6.1 were calculated using Equation 16 and the Biot coefficient (a) values were calculated using Equation 12.

After completion of the first step, the saturation process was carried out. In summary, Table 6.2 presents the obtained Skempton coefficient values.

| | | | Sample saturation | | |
|---|---|---|---|---|---|
| | | | Skempton | | Skempton |
| | Sample data | | B (First | B (Second | Cₚₚ |
| Sample | ϕ (%) | k (mD) | saturation) | saturation) | (GPa⁻¹) |
| CGS-101 | 26.78 | 676 | 0.416 | 0.422 | 0.2432 |
| CGS-102 | 26.95 | 772 | 0.398 | 0.396 | 0.2156 |
| CGS-106 | 27.0 | 911 | 0.406 | 0.410 | 0.2302 |

Since the B values are similar, the sample is considered saturated. After this verification, the step 2 test was carried out and the results are presented in Table 6.3, including the results obtained Kₛₐₜ both by the test and by the Gassmann equation.

The values of compression and shear wave velocities measured in this step are presented in FIGS. 6.7 to 6.9.

The shear wave velocity values (FIGS. 6.7 to 6.9) have small differences between them; in this case, the wave velocity values may contain additional inaccuracies due to weak signals in the tests of these samples. If this problem were in larger proportions, it could indicate that the material would have anisotropy behavior, but this is not the case. In addition, as this behavior is not expected with this material, VS1 or VS2 can be used to calculate the elastic parameters.

It is important to note that, when comparing the Vₛ values obtained from a dry sample and the same saturated sample, there is a small difference between these values, as the density of the sample is changed by the presence of water.

Table 6.3 presents a compilation of the elastic moduli values obtained from the wave propagation velocity values measured for each saturated sample at P_{c}−Pₚ equal to 6000 psi (41.369 MPa).

| | | | Dynamic tests | | | |
|---|---|---|---|---|---|---|
| | Sample data | | Saturated (Stage 2) | | | |
| | ϕ | k | E | | Kₛₐₜ (GPa) - | Kₛₐₜ (GPa) - |
| Sample | (%) | (mD) | (GPa) | ν | measured | Gassman |
| CGS-101 | 26.78 | 676 | 16.85 | 0.32 | 15.22 | 15.17 |
| CGS-102 | 26.95 | 772 | 16.84 | 0.31 | 14.87 | 14.87 |
| CGS-106 | 27.0 | 911 | 17.31 | 0.31 | 15.11 | 14.84 |

The elastic modulus values obtained in steps 1 and 2 are presented in Table 6.4 (at P_{c}−Pₚ equal to 6000 psi (41.369 MPa)). Still in the same table, the values of the measured saturated volumetric compression modulus are presented, as well as those obtained by applying the Gassmann model (Equation 38).

It is noted that the Kₛₐₜ values measured in step 2 and those calculated with fluid replacement present very similar numbers, with a maximum difference of 1.77%. As these values are similar, this is another indication that the sample saturation process worked correctly.

| | | | Dynamic tests | | | Dynamic tests | | | |
|---|---|---|---|---|---|---|---|---|---|
| | Sample data | | Dry (Stage 1) | | | Saturated (Stage 2) | | | |
| | ϕ | k | E | | K_{dry} | E | | Kₛₐₜ (GPa) - | Kₛₐₜ (GPa) - |
| Sample | (%) | (mD) | (GPa) | ν | (GPa) | (GPa) | ν | measured | Gassman |
| CGS-101 | 26.78 | 676 | 17.46 | 0.24 | 11.10 | 16.85 | 0.32 | 15.22 | 15.17 |
| CGS-102 | 26.95 | 772 | 17.20 | 0.23 | 10.71 | 16.84 | 0.31 | 14.87 | 14.87 |
| CGS-106 | 27.0 | 911 | 17.41 | 0.23 | 10.67 | 17.31 | 0.31 | 15.11 | 14.84 |

FIG. 6.10 presents the results of pore compressibility for each of the tested samples, in addition to curves calculated by linear regression indicating a trend in the behavior of Cₚₚ values for different levels of pressure difference.

### Bentheimer Sandstone (BES)

The results of tests with the proposed methodology for the three selected samples of Bentheimer sandstone (BES-101, BES-102 and BES-103) are presented.

FIGS. 6.11 to 6.13 indicate the behavior of the compression wave velocities for each of the two loading and unloading cycles of the dry sample.

As seen in the FIGS., for higher levels of confining pressure, the velocity values behave very close to the expected error (1%). Still in relation to the shape of the curve, it is worth stating that there was no damage to the samples at the stress levels applied in the tests.

Another important issue relates to the values of compression and shear wave velocities. FIGS. 6.14 to 6.16 show the values achieved.

The shear wave velocity values (FIGS. 6.14 to 6.16) presents small differences between them. If this problem were in larger proportions, it could indicate that the material would have anisotropy behavior, but this is not the case. In addition, as this behavior is not expected with this material, VS1 or VS2 can be used to calculate the elastic parameters. Wave velocity values at lower confining pressure levels may contain inaccuracies due to weak signals at these pressures.

Table 6.5 presents a compilation of the elastic and poroelastic moduli values obtained from the wave propagation velocity values measured for each sample at a confining pressure equal to 6000 psi (41.369 MPa).

| | | | Dynamic tests | | | | |
|---|---|---|---|---|---|---|---|
| | Sample data | | Dry (Stage 1) | | | | |
| | ϕ | k | E | | K_{dry} | Cₚₚ | |
| Sample | (%) | (mD) | (GPa) | ν | (GPa) | (GPa⁻¹) | α |
| BES-101 | 23.2 | 2420 | 29.48 | 0.12 | 13.05 | 0.2544 | 0.651 |
| BES-102 | 23.0 | 2420 | 29.26 | 0.13 | 13.39 | 0.2483 | 0.642 |
| BES-103 | 22.9 | 2270 | 29.92 | 0.13 | 13.49 | 0.2469 | 0.64 |

The Cₚₚ values presented in Table 6.5 were calculated using Equation 16 and the Biot coefficient (a) values were calculated using Equation 12.

After completing this step, the saturation process was carried out. In summary, Table 6.6 shows the Skempton coefficient values obtained at this point.

| | | | Sample saturation | | |
|---|---|---|---|---|---|
| | | | Skempton | | Skempton |
| | Sample data | | B (First | B (Second | Cₚₚ |
| Sample | ϕ (%) | k (mD) | saturation) | saturation) | (GPa⁻¹) |
| BES-101 | 23.2 | 2420 | 0.279 | 0.277 | 0.1145 |
| BES-102 | 23.0 | 2420 | 0.264 | 0.271 | 0.1103 |
| BES-103 | 22.9 | 2270 | 0.266 | 0.270 | 0.1096 |

As the B values are similar, the sample is considered saturated. After the verification, the step 2 test was carried out and the results are presented in Table 6.7, including the Kₛₐₜ results obtained both by the test and the Gassmann equation.

The values of the compression and shear wave velocities measured in this step are presented in FIGS. 6.17 to 6.19.

The shear wave velocity values (FIGS. 6.17 to 6.19) show small differences between them. If this problem were in larger proportions, it could indicate that the material would have anisotropy behavior, but this is not the case. In addition, as this behavior is not expected with this material, VS1 or VS2 can be used to calculate the elastic parameters.

It is important to note that, when comparing the Vₛ values obtained from a dry sample and the same saturated sample, there is a small difference between these values, as the density of the sample is changed by the presence of water.

Table 6.7 presents a compilation of the elastic moduli values obtained from the wave propagation velocity values measured for each saturated sample at P_{c}−Pₚ equal to 6000 psi (41.369 MPa).

| | | | Dynamic tests | | | |
|---|---|---|---|---|---|---|
| | Sample data | | Saturated (Stage 2) | | | |
| | ϕ | k | E | | Kₛₐₜ (GPa) - | Kₛₐₜ (GPa) - |
| Sample | (%) | (mD) | (GPa) | ν | measured | Gassman |
| BES-101 | 23.2 | 2420 | 31.89 | 0.18 | 16.48 | 17.15 |
| BES-102 | 23.0 | 2420 | 31.52 | 0.19 | 16.93 | 17.41 |
| BES-103 | 22.9 | 2270 | 32.33 | 0.18 | 16.83 | 17.50 |

The elastic modulus values obtained in steps 1 and 2 are presented in Table 6.8 (in P_{c}−Pₚ equal to 6000 psi (41.369 MPa)), as well as the saturated volumetric compression modulus values measured and those obtained by applying the Gassmann model (Equation 38).

| | | | Dynamic tests | | | Dynamic tests | | | |
|---|---|---|---|---|---|---|---|---|---|
| | Sample data | | Dry (Stage 1) | | | Saturated (Stage 2) | | | |
| | ϕ | k | E | | K_{dry} | E | | Kₛₐₜ (GPa) - | Kₛₐₜ (GPa) - |
| Sample | (%) | (mD) | (GPa) | ν | (GPa) | (GPa) | ν | measured | Gassman |
| BES-101 | 23.2 | 2420 | 29.48 | 0.12 | 13.05 | 31.89 | 0.18 | 16.48 | 17.15 |
| BES-102 | 23.0 | 2420 | 29.26 | 0.13 | 13.39 | 31.52 | 0.19 | 16.93 | 17.41 |
| BES-103 | 22.9 | 2270 | 29.92 | 0.13 | 13.49 | 32.33 | 0.18 | 16.83 | 17.50 |

The values obtained for Kₛₐₜ measured in Step 2 and those calculated with fluid replacement present similar values, with a maximum difference of 4.07%. As these values are similar, this is another indication that the sample saturation process worked correctly.

FIG. 6.20 presents the pore compressibility results obtained for each of the tested samples, in addition to curves calculated through linear regression indicating the behavior trend of Cₚₚ values for different levels of pressure difference.

### Indiana Limestone (IL3)

The test results using the proposed methodology for the three selected samples of Indiana limestone carbonate will be presented below: IL3-101, IL3-102 and IL3-106.

FIGS. 6.21 to 6.23 show the behavior of compression wave velocities for each of the two loading and unloading cycles of the dry sample.

As can be seen, for higher levels of confining pressure, the velocity values behave very close, within the expected error. Furthermore, based on the shape of the curve, it can be stated that there was no damage to the samples at the stress levels applied in the tests.

Another important issue concerns the values of compression and shear wave velocities. FIGS. 6.24 to 6.26 show these reached values.

The shear wave velocity values (FIGS. 6.24 to 6.26) show small differences between them. If this problem were in larger proportions, it could indicate that the material would have anisotropy behavior, but this is not the case. Furthermore, as this behavior is not expected with this material, VS1 or VS2 can be used to calculate the elastic parameters.

Table 6.9 presents a compilation of the elastic and poroelastic moduli values obtained from the wave propagation velocity values measured for each sample at a confining pressure equal to 6000 psi (41.369 MPa).

| | | | Dynamic tests | | | | |
|---|---|---|---|---|---|---|---|
| | Sample data | | Dry (Stage 1) | | | | |
| | ϕ | k | E | | K_{dry} | Cₚₚ | |
| Sample | (%) | (mD) | (GPa) | ν | (GPa) | (GPa⁻¹) | α |
| IL3-101 | 13.36 | 68 | 42.17 | 0.26 | 29.79 | 0.1300 | 0.574 |
| IL3-102 | 13.56 | 71 | 42.15 | 0.26 | 29.66 | 0.1290 | 0.576 |
| IL3-106 | 16.17 | 146 | 38.59 | 0.26 | 27.19 | 0.1248 | 0.611 |

The Cₚₚ values presented in Table 6.9 were calculated using Equation 16 and the Biot coefficient (a) values were calculated using Equation 12.

After completion of the first step, the saturation process was carried out. Table 6.10 below presents a summary of the achieved Skempton coefficient values.

| | | | Sample saturation | | |
|---|---|---|---|---|---|
| | | | Skempton | | Skempton |
| | Sample data | | B (First | B (Second | Cₚₚ |
| Sample | ϕ (%) | k (mD) | saturation) | saturation) | (GPa⁻¹) |
| IL3-101 | 13.36 | 68 | 0.244 | 0.247 | 0.0941 |
| IL3-102 | 13.56 | 71 | 0.236 | 0.234 | 0.0857 |
| IL3-106 | 16.17 | 146 | 0.282 | 0.290 | 0.1239 |

Since the B values are similar, the sample is considered saturated. After verification, the step 2 test was carried out, and the results are presented in Table 6.11, including the results obtained Kₛₐₜ both by the test and by the Gassmann equation.

The values of compression and shear wave velocities measured in this step are presented in FIGS. 6.27 to 6.29.

It is important to note that, when comparing the Vₛ values obtained from a dry sample and the same saturated sample, there is a small difference between these values, as the density of the sample is changed by the presence of water.

Table 6.11 presents a compilation of the elastic modulus values obtained from the wave propagation velocity values measured for each saturated sample at P_{c}−Pₚ equal to 6000 psi (41.369 MPa).

| | | | Dynamic tests | | | |
|---|---|---|---|---|---|---|
| | Sample data | | Saturated (Stage 2) | | | |
| | ϕ | k | E | | Kₛₐₜ (GPa) - | Kₛₐₜ (GPa) - |
| Sample | (%) | (mD) | (GPa) | ν | measured | Gassman |
| IL3-101 | 13.36 | 68 | 43.24 | 0.29 | 34.98 | 35.33 |
| IL3-102 | 13.56 | 71 | 43.27 | 0.29 | 34.85 | 35.16 |
| IL3-106 | 16.17 | 146 | 43.10 | 0.29 | 34.72 | 32.46 |

The values of the elastic moduli obtained in steps 1 and 2 are presented in Table 6.12 (in P_{c}−Pₚ equal to 6000 psi (41.369 MPa)), as well as the values of the saturated volumetric compression modulus measured and those obtained by applying the Gassmann model (Equation 38).

| | | | Dynamic tests | | | Dynamic tests | | | |
|---|---|---|---|---|---|---|---|---|---|
| | Sample data | | Dry (Stage 1) | | | Saturated (Stage 2) | | | |
| | ϕ | k | E | | K_{dry} | E | | Kₛₐₜ (GPa) - | Kₛₐₜ (GPa) - |
| Sample | (%) | (mD) | (GPa) | ν | (GPa) | (GPa) | ν | measured | Gassman |
| IL3-101 | 13.36 | 68 | 42.17 | 0.26 | 29.79 | 43.24 | 0.29 | 34.98 | 35.33 |
| IL3-102 | 13.56 | 71 | 42.15 | 0.26 | 29.66 | 43.27 | 0.29 | 34.85 | 35.16 |
| IL3-106 | 16.17 | 146 | 38.59 | 0.26 | 27.19 | 43.10 | 0.29 | 34.72 | 32.46 |

It can be seen that the values obtained for Kₛₐₜ measured in step 2 and those calculated with fluid replacement present similar values, with a maximum difference of 6.48%. As these values are similar, this is another indication that the sample saturation process worked correctly.

FIG. 6.30 presents the results of pore compressibility for each of the evaluated samples, in addition to curves calculated by linear regression indicating a trend in the behavior of Cₚₚ values for various levels of pressure difference.

### Silurian Dolomite (SD)

The test results using the proposed methodology for the four selected samples of Silurian Dolomite are presented below: SD-101-2nd, SD-102, SD-103 and SD-106.

FIGS. 6.31 to 6.33 show the behavior of compression wave velocities for each of the two loading and unloading cycles of the dry sample.

As can be seen, for higher levels of confining pressure, the velocity values behave very close, within the expected error. Furthermore, based on the shape of the curve, it can be stated that there was no damage to the samples at the stress levels applied in the tests.

Another important issue concerns the values of compression and shear wave velocities obtained. FIGS. 6.35 to 6.38 show these values.

Table 6.13 presents a compilation of elastic and poroelastic moduli values obtained from wave propagation velocity values measured for each sample at a confining pressure equal to 6000 psi (41.369 MPa).

The Cₚₚ values presented in Table 6.13 were calculated using Equation 16 and the Biot coefficient (a) values were calculated using Equation 12.

Since the B values are similar, the sample is considered saturated. After this verification, the step 2 test was carried out, and the results are presented in Table 6.15, including the results obtained Kₛₐₜ both by the test and by the Gassmann model (Equation 38).

The values of compression and shear wave velocities measured in this step are presented in FIGS. 6.39 to 6.42.

It is important to note that, when comparing the Vₛ values obtained from a dry sample and the same saturated sample, there is a small difference between these values, as the density of the sample is changed by the presence of water.

Table 6.15 presents a compilation of the elastic modulus values obtained from the wave propagation velocity values measured for each saturated sample at P_{c}−Pₚ equal to 6000 psi (41.369 MPa).

| | | | Dynamic tests | | | |
|---|---|---|---|---|---|---|
| | Sample data | | Saturated (Stage 2) | | | |
| | ϕ | k | E | | Kₛₐₜ (GPa) - | Kₛₐₜ (GPa) - |
| Sample | (%) | (mD) | (GPa) | ν | measured | Gassman |
| SD-101-2nd | 17.44 | 133 | 62.01 | 0.27 | 45.81 | 44.28 |
| SD-102 | 18.86 | 257 | 59.95 | 0.27 | 43.47 | 42.55 |
| SD-103 | 18.74 | 256 | 58.71 | 0.27 | 42.75 | 42.29 |
| SD-106 | 18.6 | 747 | 59.42 | 0.26 | 41.65 | 40.78 |

The values of the elastic moduli obtained in Steps 1 and 2 are presented in Table 6.16 (in P_{c}−Pₚ equal to 6000 psi (41.369 MPa)), as well as the values of the saturated volumetric compression modulus measured and those obtained by applying the Gassmann relation.

| | | | Dynamic tests | | | Dynamic tests | | | |
|---|---|---|---|---|---|---|---|---|---|
| | Sample data | | Dry (Stage 1) | | | Saturated (Stage 2) | | | |
| | ϕ | k | E | | K_{dry} | E | | Kₛₐₜ (GPa) - | Kₛₐₜ (GPa) - |
| Sample | (%) | (mD) | (GPa) | ν | (GPa) | (GPa) | ν | measured | Gassman |
| SD-101-2nd | 17.44 | 133 | 62.15 | 0.24 | 40.76 | 62.01 | 0.27 | 45.81 | 44.28 |
| SD-102 | 18.86 | 257 | 57.72 | 0.25 | 39.01 | 59.95 | 0.27 | 43.47 | 42.55 |
| SD-103 | 18.74 | 256 | 56.40 | 0.25 | 38.68 | 58.71 | 0.27 | 42.75 | 42.29 |
| SD-106 | 18.6 | 747 | 56.86 | 0.24 | 36.84 | 59.42 | 0.26 | 41.65 | 40.78 |

The values obtained for Kₛₐₜ measured in Step 2 and those calculated with fluid replacement present similar values, with a maximum difference of 3.34%. As these values are similar, this is another indication that the sample saturation process worked correctly.

FIG. 6.43 presents the pore compressibility results obtained for each of the tested samples, in addition to curves calculated by linear regression indicating a trend in the behavior of Cₚₚ values for different levels of pressure difference.

### Discussion of the Results

The main topics discussed herein concern the values of the Skempton coefficient, a brief review of the compressibility values obtained in previously published papers, as well as a comparison, when applicable, of these values with those of the new methodology.

There will be presented below the results of the pore compressibility values through empirical methods (Newman—item 2.2.3), the values obtained indirectly in the tests of the new methodology (Step 1 and those calculated from the Skempton coefficient values), as well as the values obtained directly in the tests of Step 3 of the method.

Such values will be compared with each other and with the results presented by several other authors for rocks such as those used in the invention tests.

It is worth remembering that it is common for the compressibility values obtained by dynamic tests (Step 1) to be lower than the compressibility values obtained by static tests (Step 3).

For the three Castlegate Sandstone (CGS) samples, the test results and calculated pore compressibility values (Cₚₚ) are presented in Table 6.17.

| | Sample data | | Compressibility - comparasion | | | |
|---|---|---|---|---|---|---|
| | ϕ | k | Stage 1 (Dynamic) | Stage 3 (Static) | Skempton (Static) | Newman (Empirical) |
| Sample | (%) | (mD) | Cₚₚ (GPa⁻¹) | Cₚₚ (GPa⁻¹) | Cₚₚ (GPa⁻¹) | Cₚₚ (GPa⁻¹) |
| CGS-101 | 26.78 | 676 | 0.2061 | 0.6053 | 0.2432 | 0.269 |
| CGS-102 | 26.95 | 772 | 0.2168 | 0.6050 | 0.2156 | 0.266 |
| CGS-106 | 27.0 | 911 | 0.2176 | 0.6394 | 0.2302 | 0.266 |

The compressibility value reached at the maximum level of pressure differences (P_{c}−Pₚ) was considered for Stage 1. For Stage 3, the value also corresponds to the largest pressure differences.

As can be seen, the data from the Newman solution differ in relation to the values achieved by the tests, especially when compared to the static result of Step 3 (difference of approximately 230%), indicating that these empirical data should only be used when there is no other better information and must be corrected as quickly as possible with data generated in the laboratory.

Regarding data from Phase 1 and Phase 3, the differences are relevant (around 300%) and the dynamic results are lower than the static values, which is normally expected as previously indicated. So, for this lithology, the values of the wave velocities can be used to calculate K and obtain the values of the dynamic Cₚₚ and apply a factor of 3 to achieve the static Cₚₚ, when evaluating these pore compressibilities with pressure differences (P_{c}−Pₚ) greater than 3000 psi (20.684 MPa).

FIG. 6.44 shows the curves obtained and compared with literature data.

To compare the results obtained by the proposed methodology, measurements previously carried out by Holt et al. [39] were used as the main reference. It should also be highlighted that the values presented are the volumetric compression moduli (K) and, for comparison, they were transformed into pore compressibility values (Cₚₚ) using the relations presented previously.

Such measurements were carried out through static and dynamic tests for different confining pressures, using the usual test standards. The average porosity of the samples is 29%.

It can be observed, by analyzing FIG. 6.44, that the values obtained for Cₚₚ in the static and dynamic tests using the proposed methodology and those carried out by Holt et al. [39] present similar values, which indicates that the proposed methodology presents reliable results.

For the three samples of Bentheimer sandstone (BES), test results and calculated pore compressibility values (Cₚₚ) are presented in Table 6.18.

| | Sample data | | Compressibility - comparasion | | | |
|---|---|---|---|---|---|---|
| | ϕ | k | Stage 1 (Dynamic) | Stage 3 (Static) | Skempton (Static) | Newman (Empirical) |
| Sample | (%) | (mD) | Cₚₚ (GPa⁻¹) | Cₚₚ (GPa⁻¹) | Cₚₚ (GPa⁻¹) | Cₚₚ (GPa⁻¹) |
| BES-101 | 23.2 | 2420 | 0.2544 | 0.2355 | 0.1145 | 0.5458 |
| BES-102 | 23.0 | 2420 | 0.2483 | 0.2234 | 0.1103 | 0.5502 |
| BES-103 | 22.9 | 2270 | 0.2469 | 0.2282 | 0.1096 | 0.5525 |

In this test, the compressibility value obtained at the maximum level of pressure differences (P_{c}−Pₚ) was considered for Stage 1. For Stage 3, the value also corresponds to the highest level of pressure differences.

It is observed that the data from Newman's solution differ significantly in relation to the values obtained by the tests; when compared with the result of Step 3, there is a difference of approximately 234%, corroborating that these empirical data should only be used when there is no other better information and be corrected as quickly as possible with data generated in the laboratory.

Regarding Phase 1 and Phase 3 data, the differences are small (about 7%) and dynamic results are slightly larger than static data, which is usually not expected. This situation may be due to the good distribution of the grains in the sample, due to the distribution of the sizes of the pore throats being quite uniform, without the presence of microporosities, and the mineralogical composition of the rock being the same material—97% quartz. Therefore, for this lithology, the Cₚₚ values obtained from dynamic tests can be used as static values without corrections, when evaluating these pore compressibilities with pressure differences (P_{c}−Pₚ) greater than 3000 psi (20.684 MPa).

FIG. 6.45 shows the curves compared with literature data.

To compare the values of this lithology, the C_{bc} compressibility results were used from the values measured in the laboratory by Blocher et al. [63] and converted to Cₚₚ using the relations presented previously to allow the comparison.

As observed in FIG. 6.45 when comparing the test results of the proposed methodology with the results of Blocher et al., the data for the lowest pressure differences (P_{c}−Pₚ) are not very close; however, for the lowest pressure differences high (P_{c}−P_{c}>2000 psi (13.79 MPa)), these values are closer in order of magnitude (difference of around 10% when comparing the values).

Considering that the greatest interest in knowing the behavior of pore compressibility lies in the largest pressure differences, as they configure the usual pressure values in oil reservoirs during production, the results of the proposed methodology are considered satisfactory.

The test results and pore compressibility values (Cₚₚ) are presented in Table 6.19 for the three samples of Indiana Limestone carbonate (IL3).

| | Sample data | | Compressibility - comparasion | | | |
|---|---|---|---|---|---|---|
| | ϕ | k | Stage 1 (Dynamic) | Stage 3 (Static) | Skempton (Static) | Newman (Empirical) |
| Sample | (%) | (mD) | Cₚₚ (GPa⁻¹) | Cₚₚ (GPa⁻¹) | Cₚₚ (GPa⁻¹) | Cₚₚ (GPa⁻¹) |
| IL3-101 | 13.36 | 68 | 0.1300 | 0.2156 | 0.0941 | 0.912 |
| IL3-102 | 13.56 | 71 | 0.1290 | 0.2109 | 0.0857 | 0.8994 |
| IL3-106 | 16.17 | 146 | 0.1248 | 0.5408 | 0.1239 | 0.7636 |

Above, the compressibility value obtained at the maximum level of pressure differences (P_{c}− Pₚ) was considered for Stage 1. For Stage 3, the value also corresponds to the same pressure level.

As can be seen, the data from Newman's solution differ quite significantly in relation to the values obtained by the tests; when compared to the Cₚₚ results obtained in step 3 of the proposed methodology, there is a difference of approximately 436%, corroborating that these data should only be used when there is no other better information and must be corrected as quickly as possible with data generated in the laboratory.

In relation to data from Step 1 and Step 3, there is a difference of around 65.8%. Dynamic results are smaller than static data, which is typically expected. So, for this lithology, the values of wave velocities can be used to calculate K and obtain the values of the dynamic Cₚₚ and apply a multiplication factor of 1.65 to arrive at the static Cₚₚ, when evaluating these pore compressibilities with differences in pressure (P_{c}−Pₚ) greater than 3000 psi (20.684 MPa).

FIG. 6.46 presents the curves obtained and compared with data from the literature.

For comparison purposes, compressibility values were obtained by another method developed from measurements carried out in a helium porosimeter [47]. Here, the values were only achieved up to the effective pressure value of 2000 psi (13.79 MPa) and are C values. Therefore, such data were transformed to Cₚₚ also for comparison purposes.

As can be seen in FIG. 6.46, the data measured by the new methodology and those generated by Oliveira [47] for low pressure differences (not P_{c}−Pₚ) are very close, but for larger pressure differences there is no data to be compared. It can be inferred that there is a tendency for the values at the largest pressure differences to be close to those measured by the new methodology. Again, the regions with greater pressure differences are those where there is greater interest in knowing the behavior of the pore compressibility, as these values are closer to the reality of oil reservoirs during production.

The test results and calculated pore compressibility values (Cₚₚ) are presented in Table 6.20 for the four Silurian dolomite (SD) samples.

| | Sample data | | Compressibility - comparasion | | | |
|---|---|---|---|---|---|---|
| | ϕ | k | Stage 1 (Dynamic) | Stage 3 (Static) | Skempton (Static) | Newman (Empirical) |
| Sample | (%) | (mD) | Cₚₚ (GPa⁻¹) | Cₚₚ (GPa⁻¹) | Cₚₚ (GPa⁻¹) | Cₚₚ (GPa⁻¹) |
| SD-101-2nd | 17.44 | 133 | 0.0444 | 0.2942 | 0.1329 | 0.7114 |
| SD-102 | 18.86 | 257 | 0.0458 | 0.3154 | 0.1383 | 0.6616 |
| SD-103 | 18.74 | 256 | 0.0474 | 0.2031 | 0.1329 | 0.6656 |
| SD-106 | 18.6 | 747 | 0.0548 | 0.2123 | 0.1329 | 0.6704 |

For Stage 1, the compressibility value obtained at the maximum level of pressure differences (P_{c}−Pₚ) was considered. For Stage 3, the value also corresponds to the same pressure level.

As seen in the results, the data from Newman's solution differ quite significantly in relation to the values obtained by static tests (difference of up to 241%), corroborating that these empirical data should only be used when there is no other better information and can be corrected as quickly as possible with data generated in the laboratory.

It is also worth remembering that this lithology has a relevant issue: there are pores in the samples that are not connected to each other, as well as vugs (cavities within the rock), which certainly impair the saturation of the sample. The values obtained, although relevant, are not entirely adequate.

Regarding data from Stage 1 and Stage 3, the differences are quite significant, with dynamic results being smaller than static data, which is normally expected. For this lithology and situation, the best data to use is the static data measured in Stage 3.

In FIG. 6.47, the compressibility values were obtained by another method developed from measurements carried out in a helium porosimeter [47]. In this method, values were obtained only up to the pressure differences (P_{c}−Pₚ) value of 2000 psi (13.79 MPa) and feature C_{pc} values. Therefore, such data were transformed into Cₚₚ, using Equations 14, 15 and 16 for comparison purposes.

It is worth remembering that regions with greater pressure differences are where there is greater interest in knowing the pore compressibility values, as mentioned previously.

### Results of the Biot and Skempton Coefficients

There follow the results of the Biot coefficients in each sample calculated from the test data in Stage 1 (Dynamic test). A comparison was also made of the Skempton coefficient results obtained in the tests with those calculated by poroelasticity relations. Both data for each sample are compiled in Table 6.21.

| | | | | Skempton | | |
|---|---|---|---|---|---|---|
| | Sample data | | | Calculated | Mesaured | |
| | ϕ | k | Biot | B | B (First | B (Second |
| Sample | (%) | (mD) | α | (Estimated) | Saturation) | saturation) |
| CGS-101 | 26.78 | 676 | 0.694 | 0.386 | 0.416 | 0.422 |
| CGS-102 | 26.95 | 772 | 0.705 | 0.397 | 0.398 | 0.396 |
| CGS-106 | 27.0 | 911 | 0.706 | 0.398 | 0.406 | 0.410 |
| BES-101 | 23.2 | 2420 | 0.651 | 0.366 | 0.279 | 0.277 |
| BES-102 | 23.0 | 2420 | 0.642 | 0.359 | 0.264 | 0.271 |
| BES-103 | 22.9 | 2270 | 0.640 | 0.357 | 0.266 | 0.270 |
| IL3-101 | 13.36 | 68 | 0.574 | 0.273 | 0.244 | 0.247 |
| IL3-102 | 13.56 | 71 | 0.576 | 0.271 | 0.236 | 0.234 |
| IL3-106 | 16.17 | 146 | 0.611 | 0.265 | 0.282 | 0.290 |
| SD-101- | 17.44 | 133 | 0.508 | 0.156 | 0.298 | 0.302 |
| 2nd | | | | | | |
| SD-102 | 18.86 | 257 | 0.529 | 0.157 | 0.315 | 0.309 |
| SD-103 | 18.74 | 256 | 0.533 | 0.160 | 0.299 | 0.302 |
| SD-106 | 18.6 | 747 | 0.556 | 0.173 | 0.300 | 0.302 |

The obtained Biot coefficient values serve as a first approximation and were calculated using Equation 12, the K_{g} from Table 5.5 and K_{dry} measured in Step 1 of the experimental methodology.

For a more accurate evaluation of the Biot coefficient, a static test must be carried out to obtain the value of the rock's volumetric compressibility modulus and grain compressibility values measured for each sample (not the values in Table 5.4).

However, the Biot values are relevant when the material is within the elastic regime, and the dynamic tests performed are also within this regime.

Regarding the Skempton coefficients (B) measured compared to the poroelasticity solution (Equation 18), it is worth highlighting that, in Equation 18, the following parameters were considered for calculating B: the Biot coefficient values from Table 6.21, volumetric compressibility modulus values obtained in Step 1 (dynamic tests) and the grain compressibility values from Table 5.4 for each sample. On the other hand, the B was obtained in the experiment by using the static method, since the effect of increasing pore pressure was measured directly as a function of increasing confining pressure, as described in section 3.1.2.

Therefore, these results must be compared carefully, as there are dynamic and static measurements involved. Even so, it can be noted that, when comparing the B values measured for the Castlegate Sandstone and Indiana Limestone samples, they are very close to those measured in relation to the poroelasticity of the solution (maximum difference of 10%). However, the B values for the Bentheimer Sandstone (BES) and the Silurian Dolomite (SD) are different from those obtained by Equation 18, a difference of up to 30% for the BES samples and up to 90% for the SD samples.

Regarding the B values of the SD samples, it can be inferred that there is an influence of the characteristic porosity of the rock, as it has vugs and saturation cannot occur in these spaces.

Those skilled in the art will value the knowledge presented herein and will be able to reproduce the invention in the presented embodiments and in other variants, encompassed by the scope of the attached claims.

### List of Abbreviations, Symbols and Acronyms

### Roman and Latin Characters

B Skempton coefficient
C_{bc} Bulk volume compressibility varying the confining pressure
C_{bc,uni} Bulk volume compressibility varying the confining pressure—uniaxial deformation
C_{bp} Bulk volume compressibility varying the pore pressure
C_{bp,uni} Bulk volume compressibility varying the pore pressure—uniaxial deformation
C_{f} Compressibility of fluids
C_{g} Compressibility of rock grains
C_{pc} Pore compressibility varying the confining pressure
C_{pc,uni} Pore compressibility varying the confining pressure—uniaxial deformation
Cₚₚ Pore compressibility varying the confining pressure
C_{pp,uni} Pore compressibility varying the pore pressure —uniaxial deformation
E Young's modulus
G Shear modulus
K Volumetric compression module
K_{dry} Volumetric compression module—dry rock
Kᵢ Volumetric compression modulus of the mineral i
K_{R} Volumetric compression modulus—Reuss average
K_{V} Volumetric compression modulus—Voigt average
K_{VRH} Volumetric compression module—Voigt-Reuss-Hill average
Kₛₐₜ Volumetric compression modulus—saturated rock
K_{g} Volumetric modulus of the rock component
K_{fl} Volumetric modulus of fluid
l Sample length
P_{c} Confining pressure
Pₚ Pore pressure
R Factor that correlates the vertical deformation with the variation in time shift between two seismic surveys
t Total wave time shift
t₀ System time
V_{b} Bulk Volume—Total volume of the rock
V_{equipment} Equipment volume (pump, lines, valves and pressure vessel)
V_{fluid} Volume of fluid present in the system (pump, lines, sample pores)
V_{P} Compression wave propagation velocity
Vₚₒᵣₑ Pore volume—part of the Bulk volume that is not occupied by rock minerals
V_{prod} Volume of produced oil
V_{S} Shear wave propagation velocity
V_{system} Volume read at the pressure pump
Vᵣ Volume of the grains that form the rock

### Greek Characters

α Biot-Willis coefficient
ε Deformation
ε_{b} Deformation related to the volume of the rock
εₚₒᵣₑ Pore deformation related to the rock pore
ε_{zz} Vertical deformation of the reservoir
ϕ Porosity
μ_{dry} Shear modulus for dry rock
μₛₐₜ For saturated rock
ν Poisson's ratio
ρ Density
ρ_{g} Density of the solid part of the rock
ρ_{fl} Density of the fluid in the rock
σ Stress
σ_{eff} Effective stress

### Bibliographic References

[1] Fjaer, E.; Holt, R. M.; Raaen, A. M.; Risnes, R. and Horsrud, P. Petroleum Related Rock Mechanics, Developments in Petroleum Science-Volume 53, 2nd Edition Elsevier Science, 2008.
[2] Vasquez, G.; Morschbacher, M.; Justen, J.; Figueiredo, M. and Lacerda, D. “Poroelasticity of the Pre-salt Reservoir Rocks”. 16th International Congress of the Brazilian Geophysical Society, pp. 1-6, 2019.
[3] Zimmerman, R. W. Compressibility of Sandstones, 1st edition New York: Elsevier Science, 1991.
[4] Chertov, M. A. and Suarez-Rivera, R. “Practical Laboratory Methods for Pore Volume Compressibility Characterization in Different Rock Types”, 48th US Rock Mechanics Symposium, 2014.
[5] Dudley, J. W. et al., “ISRM Suggested Method for Uniaxial-Strain Compressibility Testing for Reservoir Geomechanics”, Rock Mechanics and Rock Engineering, vol. 49, no. 10, pp. 4153-4178, 2016.
[6] Blöcher, G.; Reinsch, T.; Hassanzadegan, A.; Milsch, H. and Zimmermann, G. “Direct and indirect laboratory measurements of poroelastic properties of two consolidated sandstones”, International Journal of Rock Mechanics and Mining Sciences, vol. 67, pp. 191-201, 2014.
[7] Geertsma, J. “Problems of rock mechanics In petroleum production engineering”, 1st ISRM Congress, pp. 385-393, 1966.
[8] W. Zimmerman, W. H. Somerton, and M. S. King, “Compressibility of Porous Rocks”, Journal of Geophysics Research, vol. 91, no. B12, pp. 12,765-12,777,1986.
[9] Wang, H. F. Theory of linear poroelasticity: with applications to geomechanics and hydrogeology, 1st Edition, New Jersey: Princeton University Press, 2000.
[10] Zimmerman, R. W. “Coupling in poroelasticity and thermoelasticity”, International Journal of Rock Mechanics and Mining Sciences, vol. 37, pp. 79-87, 2000.
[11] Goodman, R. E. Introduction to Rock Mechanics, 2nd Edition John Wiley & Sons, 1989.
[12] Rosa, A. L. R. Análise do Sinal Sísmico, Segunda Ed. Rio de Janeiro: Sociedade Brasileira de Geofísica-SBGf, 2018.
[13] Zimmerman, R. W. “Pore Volume and Porosity Changes under Uniaxial Strain Conditions”, Transport in Porous Media, vol. 119, pp. 481-498, 2017.
[14] Alam, M. M. et al., “Biot's coefficient as an indicator of strength and porosity reduction: Calcareous sediments from Kerguelen Plateau”, Journal of Petroleum Science and Engineering, vol. 70, no. 3-4, pp. 282-297, 2010.
[15] Ingraham, M. D.; Bauer, S. J.; Issen, K. A. and Dewers, T. A. “Evolution of permeability and Biot coefficient at high mean stresses in high porosity sandstone”, International Journal of Rock Mechanics and Mining Sciences, vol. 96, pp. 1-10, 2017.
[16] Newman, G. H. “Pore-Volume Compressibility of Consolidated, Friable, and Unconsolidated Reservoir Rocks Under Hydrostatic Loading”, Journal of Petroleum Technology, vol. 25, pp 129-134, 1973.
[17] Hall, H. N. “Compressibility of Reservoir Rocks”, Petroleum Transactions, vol. 198, pp. 309-311, 1953.
[18] Baker, J.; Yarraton, R.; Jensen, H.; Practical Reservoir Engineering and Characterization, 1st Edition. New York: Elsevier, 2015.
[19] S Rosa, A. J.; Carvalho, R. S. and Xavier, J. A. D. Engenharia de Reservatórios de Petróleo, la edição. Rio de Janeiro: Editora Interciência, 2006.
[20] Falcão, F. O. L. “Simulação Hidromecânica de Reservatório Carbonatico de Petróleo através de Pseudoacoplamento”, Tese-Pontifícia Universidade Católica do Rio Janeiro, Departamento de Engenharia Civil, 2013.
[21] Samier, P.; Onaisi, A. and Fontaine, G. “Coupled Analysis of Geomechanics and Fluid Flow in Reservoir Simulation”, in SPE Reservoir Simulation Symposium, pp. 1-16, 2003.
[22] Garcia, P. F. V.; Rossi, D. F.; Soares, A. C.; Ferreira, F. H. and Lonardelli, J. do N. “An experimental approach on calcarenite reservoir dilation factor”, Leading Edge, vol. 40, no. 12, pp. 931-935, 2021.
[23] MacBeth, C.; Mangriotis, M. D. and Amini, H. “Review Paper: Post-stack 4D seismic time-shifts: Interpretation and evaluation”, Geophysics Prospecting, vol. 67, no. 1, pp. 3-31, 2019.
[24] Dias, A. G.; Freitas, M. C.; Guedes, F. and Bastos, M. C. “Estrutura interna da Terra”, Revista de Ciência Elementar-USP, vol. 1, no. 1, 2013.
[25] Schön, J. H. Propriedades físicas das rochas aplicadas à Engenharia de Petróleo, la Edição. Rio de Janeiro: GEN LTC, 2014.
[26] Mavko, G.; Mukerji, T. and Dvorkin, J. The Rock Physics Handbook, 1st edition Cambridge: Cambridge University Press, 2009.
[27] Gassmann, F. “On Elasticity of Porous Media by F. Gassmann, Zürich, 1951”, in Classics of Elastic Wave Theory, Zurich: Society of Exploration Geophysicists, 2007, pp. 389-408.
[28] Wang, Z. Z. “Y2K Tutorial Fundamentals of seismic rock physics”, Geophysics, vol. 66, no. 2, pp. 398-412, 2001.
[29] Ledbetter, H. “Letter Dynamic vs. static Young's moduli: a case study”, Material Science and Engineering, vol. A, no. 165, pp. L9-L10, 1993.
[30] Simmons, G and Brace, W. F. “Comparison of static and dynamic measurements of compressibility of rocks”, Journal of Geophysical Research, vol. 70, no. 22, pp. 5649-5656, 1965.
[31] King, M. S. “Static and dynamic elastic moduli of rocks under pressure”, 11th US Symposium of Rock Mechanics, pp. 320-351, 1969.
[32] Cheng, C. H. and Johnston, D. H. “Dynamic and static moduli”, Geophysical Research Letters, vol. 8, no. 1, pp. 39-42, 1981.
[33] Van Heerden, W. L. “General Relations Between Static and Dynamic Moduli of Rocks”, Journal of Rock Mechanics, Mining Sciences and Geomechanics, vol. 24, no. 6, pp. 381-385, 1987.
[34] Eissa, E. A. and Kazi, A. “Relation between static and dynamic Young's moduli of rocks”, Journal of Rock Mechanics, Mining Sciences and Geomechanics, vol. 25, no. 6, pp. 479-482, 1988.
[35] Plona, T. J. and Cook, J. M. “Effects of stress cycles on static and dynamic Young's moduli in Castlegate sandstone”, The 35th U.S.A. Symposium on Rock Mechanics, pp 155-160, 1995.
[36] Fjaer, E. “Static and dynamic moduli of a weak sandstone”, Geophysics, vol. 74, no. 2, pp. WA103-WA112, 2009.
[37] Holt, R. M.; Fjaer, E. and Bauer, A. “Static and Dynamic Moduli-so equal, and yet so different”, 47th US Rock Mechanics Symposium, p. 8, 2013.
[38] Holt, R. M.; Kjølaas, J.; Larsen, I.; Gotusso Pillitteri, L. Li, A. and Sønstebø, E. F. “Comparison between controlled laboratory experiments and discrete particle simulations of the mechanical behavior of rock”, International Journal of Rock Mechanics and Mining Sciences, vol. 42, no. 7-8 SPEC. ISS., pp. 985-995, 2005.
[39] Fjaer, E.; Stroisz, A. M. and Holt, R. M. “Static Versus Dynamic Moduli: Another Piece in the Puzzle”, 49th US Rock Mechanics Geomechanics Symposium, vol. 4, pp. 3126-3131, 2015.
[40] Duffy, J. R. D. M., “Stress-strain relations and vibrations of a granular medium.”, Journal of Applied Mechanics, vol. 24, pp. 585-593, 1957.
[41] Yamamuro, J. A.; Bopp, P. A. and Lade, P. V. “One-Dimensional Compression of Sands at High Pressures”, Journal of Geotechnical Engineering, vol. 122, no. 2, pp. 147-154, 1996.
[42] Stoll, R. D. “Stress-induced anisotropy in sediment acoustics”, The Journal of the Acoustical Society of America, vol. 82, no. S1, pp. S111-S111, November 1987.
[43] Yan, F.; Han, D.-H.; Ren, J.; Wang, Y. and. Chen, X.-L “Comparison of dynamic and static bulk moduli of reservoir rocks”, SEG Technical Program Expanded Abstracts, pp. 3711-3715, 2017.
[44] Bilal, A.; Myers, M. and Hathon, L. “An investigation of static and dynamic data using multistage triaxial tests”, SEG Technical Program Expanded Abstracts, pp. 3483-3486, 2016.
[45] Armaghani, D. J.; Amin, M. F. M.; Yagiz, S.; Faradonbeh, R. S. and Abdullah, R. A. “Prediction of the uniaxial compressive strength of sandstone using various techniques”, International modeling Journal of Rock Mechanics and Mining Sciences, Vol. 85, pp 174-186, 2016.
[46] Onalo, D.; Oloruntobi, O.; Adedigba, S.; Khan, F.; James, L. and Butt, S. “Static Young's modulus model prediction for formation evaluation”, Journal of Petroleum Science and Engineering, vol. 171, pp. 394-402, 2018.
[47] de Oliveira, G. L. P. et al., “Pore volume compressibilities of sandstones and carbonates from Helium porosimetry measurements”, Journal of Petroleum Science and Engineering, vol. 137, pp. 185-201, 2016.
[48] Justen, J. C. R. “Análise das propriedades elásticas de rochas e minerais evaporíticos”, PUC-Rio Dissertação de Mestrado, pp. 1-121, 2014.
[49] Morschbacher, M. J., Vasquez, G. F. and Justen, J. C. “Metodologias De Estimativa Das Velocidades Sísmicas Em Ensaios De Laboratório”, in IV Simpósio Brasileiro de Geofísica, 2010, pp. 1-3.
[50] Makhnenko, R. Y. and Labuz, J. F. “Saturation of porous rock and measurement of the B coefficient”, 47th US Rock Mechanics/Geomechanics Symposium, vol. 1, pp. 679-684, 2013.
[51] ASTM, “D 2845-08-Standard Test Method for Laboratory Determination of Pulse Velocities and Ultrasonic Elastic Constants of Rock”, pp. 1-7, 2008.
[52] S Vasquez, G. F.; Morschbacher, M. J.; Justen, J. c. and Lacerda, D. “Petroelasticity: Assessing Rock Properties under Stress through (Often) Non-Destructive Tests”, in 13th International Congress of the Brazilian Geophysical Society, 2013, no. 2008, pp. 1024-1029.
[53] Jaeger, J. C.; Cook, N. G. W. and Zimmerman, R. W. Fundamentals of Rock Mechanics, 4th Edition Oxford: Blackwell Publishing Ltd, 2007.
[54] Bishop, A. W. “The influence of system compressibility on the observed pore-pressure response to an undrained in stress in saturated rock”, Geotechnique, vol. 26, no. 2, pp. 371-375, 1976.
[55] Laurent, J.; Bouteca, M. J.; Sarda, J. P. and Bary, D. “Pore-pressure influence in the poroelastic behavior of rocks: experimental studies and results”, SPE Formation Evaluation, vol. 8, no. 2, pp. 117-122, 1993.
[56] McPhee, C.; Reed, J. and Zubizarreta, I. Core Analysis: A best practice guide-Developments in Petroleum Science, vol. 64, pp. 1-15, 2015.
[57] McPhee, C.; Reed, J. and Zubizarreta, I. Core Sample Preparation-Developments in Petroleum Science, vol. 64, no. 1988. 2015.
[58] Silva, Y. M. P. “Métodos avançados para caracterização de mineralogia, porosidade e de parâmetros petrofísicos em reservatórios carbonáticos”, Dissertação de Mestrado-Universidade Federal do Rio de Janeiro, Museu Nacional, 2019.
[59] API, “Recommended practices for core analysis”, API Recomm. Pract., no. 40 ED. 2 REV., 1998.
[60] Giesche, H. “Mercury porosimetry: A general (practical) overview”, Particle and Particle Systems Characterization, vol. 23, no. 1, pp. 9-19, 2006.
[61] Wang, H. F. Theory of Linear Poroelasticity with Applications to Geomechanics and Hydrogeology, 1st Edition New Jersey: Princeton University Press, 2001.
[62] Batzle, M. and Wang, Z. “Seismic properties of pore fluids”, Geophysics, vol. 57, no. 11, pp. 1396-1408, 1992.
[63] Blocher, G.; Reinsch, T.; Hassanzadegan, A.; Milsch, H. and Zimmermann, G. “Direct and indirect laboratory measurements of poroelastic properties of two consolidated sandstones”, International Journal of Rock Mechanics and Mining Sciences, vol. 67, pp. 191-201, 2014.

1. A method for determining static and dynamic elastic and poroelastic parameters of rocks under hydrostatic confining pressure conditions, comprising:
in a first step, carrying out a first dynamic test with a dry sample inside a pressure vessel;
in a second step, said dry sample is saturated with deaerated water and a second dynamic test is carried out with the saturated sample and constant pore pressure; and
in a third step, carrying out a third static compressibility test on said saturated sample.

2. The method according to claim 1, wherein the first step additionally comprises the steps of:
a) applying a loading and unloading cycle, at predefined pressure increments, over a confining pressure range Pₚ with loading and unloading paths;
b) obtaining values of V_{P} and V_{S} for each of the predefined pressure increments of the confining pressure Pₚ in the cycle;
c) calculating the elastic parameters E_{dry}, v_{dry} and K_{dry} and the poroelastic parameter Cₚₚ of the cycle;
d) repeating steps a) to c) to obtain the same parameters as steps b) and c); and
e) comparing the results of the first and second velocity cycles to check the integrity of the sample.

3. The method according to claim 2, wherein the initial P_{c} value is equal to the atmospheric pressure.

4. The method according to claim 2, wherein step a) additionally comprises varying the hydrostatic confining pressure between 0 and 6000 psi (41.369 MPa), in increments ΔP_{c} of 500 psi (3.447 MPa).

5. The method according to claim 2, wherein, in step e), the integrity check comprises checking whether the sample is in the elastic regime.

6. The method according to claim 1, wherein the second step additionally comprises the steps of:
a) saturating the dry sample inside the pressure vessel;
b) in a first flow operation, flowing a first predefined amount of pore volumes through the sample;
c) running a first Skempton test and calculating a first parameter B;
d) in a second flow operation, flowing a second predefined amount of pore volumes through the sample;
e) running a second Skempton test and calculating a second parameter B;
f) checking the measured values of Skempton B from steps c) and e) and comparing the same; if the Skempton parameters B have values within an acceptable range of difference, the sample is considered saturated;
g) varying the confining pressure range, in predefined pressure increments with loading and unloading paths, and maintaining a constant pore pressure Pₚ;
h) obtaining values of V_{P} and V_{S} for each of the predefined pressure increments of the confining pressure Pₚ in the cycle; and
i) calculating the elastic parameters Eₛₐₜ, vₛₐₜ and Kₛₐₜ, and the poroelastic parameter Cₚₚ for each of the predefined pressure increments of the cycle.

7. The method according to claim 6, wherein the dynamic tests begin while maintaining a constant pore pressure Pₚ of 1000 psi (6.895 MPa).

8. The method according to claim 6, wherein step b) comprises flowing at least 5 pore volumes through the sample.

9. The method according to claim 6, wherein step d) comprises flowing at least 5 pore volumes through the sample.

10. The method according to claim 6, wherein, in step f), the sample is considered saturated if the Skempton parameters B have values within ±5% of difference.

11. The method according to claim 6, wherein step g) comprises varying the confining pressure P_{c} from 1500 to 7000 psi (10.342 to 48.263 MPa), with increments of 500 psi (3.447 MPa), while maintaining the pore pressure Pₚ at 1000 psi (6.895 MPa).

12. The method according to claim 1, wherein the third step additionally comprises the steps of:
a) simultaneously increasing the confining pressure and the pore pressure while maintaining a predefined pressure difference between the values of P_{c} and Pₚ;
b) maintaining the confining pressure constant;
c) varying the pore pressure from a predetermined maximum pressure level and then decreasing the pressure at a predefined rate to a predetermined minimum pressure value;
d) measuring pore volume changes ΔVₚₒᵣₑ during the test;
e) obtaining curves of pressure variation×volume variation of the system V_{fluid};
f) calculating the variation in pore volume Vₚₒᵣₑ considering the effects of the system; and
g) obtaining the pore compressibility Cₚₚ for the pressure variation range presented in step c).

13. The method according to claim 12, wherein, in step a), the differential pressure is at least 200 psi (1.379 MPa).

14. The method according to claim 12, wherein, in step b), the constant confining pressure is 6000 psi (41.369 MPa).

15. The method according to claim 12, wherein, in step c), the maximum pore pressure level is 5800 psi (39.99 MPa) and the minimum pore pressure level is 1000 psi (6.895 MPa).

16. A system for carrying out the method, as defined in claim 1, wherein said system comprises, at least:
two syringe pumps;
a pressure vessel, comprising two transducers located inside the same, for confining a sample between them;
an oscilloscope;
a pulse generator; and
a computing system.

17. The system according to claim 16, wherein the system further comprises electrical and mechanical connections necessary to run tests.

18. The system according to claim 16, wherein one of the syringe pumps serves to apply a pore pressure and the other syringe pump serves to apply a confining pressure.

19. The system according to claim 16, wherein the core support can be a hydraulic vessel.

20. The system according to claim 16, wherein the transducers are acoustic velocity transducers, wherein one is for transmitting ultrasonic pulses and the other for receiving said ultrasonic pulses.

21. A computer-readable storage medium, wherein it contains a set of instructions that, when executed, carry out the method as defined in claim 1.
